SEQUENCE LISTING (43 sequences)

SEQ ID NO: 1 - protein, 5 aa, Homo sapiens
NYWMH

SEQ ID NO: 2 - protein, 17 aa, Homo sapiens
GISGWSSWTYYADSVKG

SEQ ID NO: 3 - protein, 13 aa, Homo sapiens
FGIDAYTKVYFDY

SEQ ID NO: 4 - protein, 11 aa, Homo sapiens
SGDNIPLKYVS

SEQ ID NO: 5 - protein, 7 aa, Homo sapiens
DDNKRPS

SEQ ID NO: 6 - protein, 11 aa, Homo sapiens
SSWDTLDIFNV

SEQ ID NO: 7 - DNA, 366 bp, Homo sapiens
  CDS (1)..(366)
caggtggaattggtggaaagcggcggcggcctggtgcaaccgggcggcagcctgcgtctgagctgcgcggcctccggatttaccttttctaattattggatgcattgggtgcgccaagcccctgggaagggtctcgagtgggtgagcggtatctctggttggtctagctggacctattatgcggatagcgtgaaaggccgttttaccatttcacgtgataattcgaaaaacaccctgtatctgcaaatgaacagcctgcgtgcggaagatacggccgtgtattattgcgcgcgttttggtattgatgcttatactaaggtttattttgattattggggccaaggcaccctggtgacggttagctca

SEQ ID NO: 8 - protein, 122 aa, Homo sapiens
QVELVESGGGLVQPGGSLRLSCAASGFTFSNYWMHWVRQAPGKGLEWVSGISGWSSWTYYADSVKGRFTISRDNSKNTLYLQMNSLRAEDTAVYYCARFGIDAYTKVYFDYWGQGTLVTVSS

SEQ ID NO: 9 - DNA, 327 bp, Homo sapiens
  CDS (1)..(327)
gatatcgaactgacccagccgccttcagtgagcgttgcaccaggtcagaccgcgcgtatctcgtgtagcggcgataatattcctcttaagtatgtttcttggtaccagcagaaacccgggcaggcgccagttcttgtgattcatgatgataataagcgtccctcaggcatcccggaacgctttagcggatccaacagcggcaacaccgcgaccctgaccattagcggcactcaggcggaagacgaagcggattattattgctcttcttgggatactcttgatatttttaatgtgtttggcggcggcacgaagttaaccgtcctaggt

SEQ ID NO: 10 - protein, 109 aa, Homo sapiens
DIELTQPPSVSVAPGQTARISCSGDNIPLKYVSWYQQKPGQAPVLVIHDDNKRPSGIPERFSGSNSGNTATLTISGTQAEDEADYYCSSWDTLDIFNVFGGGTKLTVLG

SEQ ID NO: 11 - protein, 5 aa, Homo sapiens
NYWMH

SEQ ID NO: 12 - protein, 17 aa, Homo sapiens
GISGWSSWTYYADSVKG

SEQ ID NO: 13 - protein, 13 aa, Homo sapiens
FGIDAYTKVYFDY

SEQ ID NO: 14 - protein, 11 aa, Homo sapiens
SGDNIPLKYVS

SEQ ID NO: 15 - protein, 7 aa, Homo sapiens
DDNKRPS

SEQ ID NO: 16 - protein, 11 aa, Homo sapiens
QSYDYFPKFVV

SEQ ID NO: 17 - DNA, 366 bp, Homo sapiens; identical to SEQ ID NO: 7
  CDS (1)..(366)

SEQ ID NO: 18 - protein, 122 aa, Homo sapiens; identical to SEQ ID NO: 8

SEQ ID NO: 19 - DNA, 327 bp, Homo sapiens
  CDS (1)..(327)
gatatcgaactgacccagccgccttcagtgagcgttgcaccaggtcagaccgcgcgtatctcgtgtagcggcgataatattcctcttaagtatgtttcttggtaccagcagaaacccgggcaggcgccagttcttgtgattcatgatgataataagcgtccctcaggcatcccggaacgctttagcggatccaacagcggcaacaccgcgaccctgaccattagcggcactcaggcggaagacgaagcggattattattgccagtcttatgattattttcctaagtttgttgtgtttggcggcggcacgaagttaaccgtcctaggt

SEQ ID NO: 20 - protein, 109 aa, Homo sapiens
DIELTQPPSVSVAPGQTARISCSGDNIPLKYVSWYQQKPGQAPVLVIHDDNKRPSGIPERFSGSNSGNTATLTISGTQAEDEADYYCQSYDYFPKFVVFGGGTKLTVLG

SEQ ID NO: 21 - protein, 5 aa, Homo sapiens
SYWMS

SEQ ID NO: 22 - protein, 17 aa, Homo sapiens
SITSYGSFTYYADSVKG

SEQ ID NO: 23 - protein, 8 aa, Homo sapiens
NMYTHFDS

SEQ ID NO: 24 - protein, 13 aa, Homo sapiens
SGSSSNIGSNSVS

SEQ ID NO: 25 - protein, 7 aa, Homo sapiens
DNSKRPS

SEQ ID NO: 26 - protein, 11 aa, Homo sapiens
QSRDTYGYYWV

SEQ ID NO: 27 - DNA, 351 bp, Homo sapiens
  CDS (1)..(351)
caggtggaattggtggaaagcggcggcggcctggtgcaaccgggcggcagcctgcgtctgagctgcgcggcctccggatttacctttacttcttattggatgtcttgggtgcgccaagcccctgggaagggtctcgagcttgtgagctctatcacttcttatggtagctttacctattatgcggatagcgtgaaaggccgttttaccatttcacgtgataattcgaaaaacaccctgtatctgcaaatgaacagcctgcgtgcggaagatacggccgtgtattattgcgcgcgtaatatgtatactcattttgattcttggggccaaggcaccctggtgacggttagctca

SEQ ID NO: 28 - protein, 117 aa, Homo sapiens
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSS

SEQ ID NO: 29 - DNA, 333 bp, Homo sapiens
  CDS (1)..(333)
gatatcgtgctgacccagccgccttcagtgagtggcgcaccaggtcagcgtgtgaccatctcgtgtagcggcagcagcagcaacattggttctaattctgtgtcttggtaccagcagttgcccgggacggcgccgaaacttctgatttatgataattctaagcgtccctcaggcgtgccggatcgttttagcggatccaaaagcggcaccagcgcgagccttgcgattacgggcctgcaaagcgaagacgaagcggattattattgccagtctcgtgatacttatggttattattgggtgtttggcggcggcacgaagttaaccgtcctaggt

SEQ ID NO: 30 - protein, 111 aa, Homo sapiens
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLG

SEQ ID NO: 31 - DNA, 993 bp, Homo sapiens
  CDS (1)..(993)
gcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 32 - protein, 330 aa, Homo sapiens [sequence omitted]

SEQ ID NO: 33 - DNA, 318 bp, Homo sapiens
  CDS (1)..(318)
cagcccaaggctgccccctcggtcactctgttcccgccctcctctgaggagcttcaagccaacaaggccacactggtgtgtctcataagtgacttctacccgggagccgtgacagtggcctggaagggagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 34 - protein, 105 aa, Homo sapiens
QPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 35 - protein, 452 aa, Homo sapiens
QVELVESGGGLVQPGGSLRLSCAASGFTFSNYWMHWVRQAPGKGLEWVSGISGWSSWTYYADSVKGRFTISRDNSKNTLYLQMNSLRAEDTAVYYCARFGIDAYTKVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, Homo sapiens
DIELTQPPSVSVAPGQTARISCSGDNIPLKYVSWYQQKPGQAPVLVIHDDNKRPSGIPERFSGSNSGNTATLTISGTQAEDEADYYCSSWDTLDIFNVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 37 - protein, 452 aa, Homo sapiens; identical to SEQ ID NO: 35

SEQ ID NO: 38 - protein, 214 aa, Homo sapiens
DIELTQPPSVSVAPGQTARISCSGDNIPLKYVSWYQQKPGQAPVLVIHDDNKRPSGIPERFSGSNSGNTATLTISGTQAEDEADYYCQSYDYFPKFVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 39 - protein, 447 aa, Homo sapiens
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 216 aa, Homo sapiens
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 41 - protein, 17 aa, Homo sapiens
LCGAELVDALQFVCGDR

SEQ ID NO: 42 - protein, 13 aa, Homo sapiens
CCFRSCDLRRLEM

SEQ ID NO: 43 - protein, 70 aa, Homo sapiens
GPETLCGAELVDALQFVCGDRGFYFNKPTGYGSSSRRAPQTGIVDECCFRSCDLRRLEMYCAPLKPAKSA

(19) United States
(12) Patent Application Publication
(10) Pub. No.: US 2013/0122005 A1
(43) Pub. Date: May 16, 2013

(54) ANTICANCER COMBINATION THERAPY

(71) Applicants: Paul ADAM, Vienna (AT); Patrizia SINI, Brunn am Gebirge (AT)

(72) Inventors: Paul ADAM, Vienna (AT); Patrizia SINI, Brunn am Gebirge (AT)

(21) Appl. No.: 13/661,473

(22) Filed: Oct. 26, 2012

(30) Foreign Application Priority Data
Oct. 27, 2011 (EP) 11186999.6

(51) Int. Cl. A61K 39/395; A61K 31/4045; A61K 31/496

(52) U.S. Cl. CPC A61K 39/3955; A61K 31/496; A61K 31/4045
USPC 424/135.1; 424/139.1

(57) ABSTRACT

The invention describes anti-cancer therapies comprising using a dual Aurora kinase/MEK inhibitor and an anti-IGF antibody, each as described herein.

The invention relates to methods for the treatment and/or prevention of oncological or hyperproliferative diseases, in particular cancer, comprising the combined administration of a dual Aurora kinase/MEK inhibitor and an anti-IGF antibody, as well as to pharmaceutical compositions or combinations comprising such active ingredients.

Further, the invention relates to anti-cancer therapies comprising using a dual Aurora kinase/MEK inhibitor and an anti-IGF antibody, each as described herein, in combination.

For the treatment of diseases of oncological nature, a large number of anticancer agents (including target-specific and non-target-specific anticancer agents) have already been suggested, which can be used as monotherapy or as combination therapy involving more than one agent (e.g. dual or triple combination therapy) and/or which may be combined with radiotherapy (e.g. irradiation treatment), radio-immunotherapy and/or surgery.

Even if the concept of combining several therapeutic agents or therapies already has been suggested, and although various combination therapies are under investigation and in clinical trials, there is still a need for new and efficient therapies of cancer diseases, which show advantages over standard therapies, such as for example better treatment outcome, beneficial effects, superior efficacy and/or improved tolerability, such as e.g. reduced side effects of the combined treatment.

It is a purpose of the present invention to provide combination therapies with the active agents described herein for treating or controlling various malignancies (e.g. based on cooperative, complementary, interactive or improving effects of the active components involved in combination).

Thus, in one aspect, the invention provides a method of treating and/or preventing an oncological or hyperproliferative disease, in particular cancer, comprising administering to a patient in need thereof a therapeutically effective amount of a dual Aurora kinase/MEK inhibitor and an anti-IGF antibody, each as described herein.

Such a combined treatment may be given as a free combination of the substances or in the form of a fixed combination, including kit-of-parts.

In another aspect, the invention refers to a combination of a dual Aurora kinase/MEK inhibitor and an anti-IGF antibody, each as described herein, particularly for use in method of treating and/or preventing an oncological or hyperproliferative disease, in particular a cancer disease e.g. as described herein, said method comprising administering to a patient in need thereof a therapeutically effective amount of the combination.

In another aspect, the invention refers to a dual Aurora kinase/MEK inhibitor as described herein for use in a method of treating and/or preventing an oncological or hyperproliferative disease, in particular cancer, said method comprising administering the dual Aurora kinase/MEK inhibitor in combination with an anti-IGF antibody as described herein to the patient in need thereof.

In another aspect, the invention refers to an anti-IGF antibody as described herein for use in a method of treating and/or preventing an oncological or hyperproliferative disease, in particular cancer, said method comprising administering the anti-IGF antibody in combination with a dual Aurora kinase/MEK inhibitor as described herein to the patient in need thereof.

In another aspect, the invention refers to a kit including a first pharmaceutical composition or dosage form which comprises a dual Aurora kinase/MEK inhibitor as described herein, and a second pharmaceutical composition or dosage form which comprises an anti-IGF antibody as described herein.

In another aspect, the invention refers to a pharmaceutical compsition containing a dual Aurora kinase/MEK inhibitor as described herein, an anti-IGF antibody as described herein, and, optionally, one or more pharmaceutically acceptable carriers, excipients and/or vehicles.

In another aspect, the invention refers to a combination comprising a dual Aurora kinase/MEK inhibitor as described herein, and an anti-IGF antibody as described herein, e.g. for simultaneous, concurrent, sequential, successive, alternate or separate use in therapy.

In another aspect, the invention refers to the use of a dual Aurora kinase/MEK inhibitor as described herein for preparing a pharmaceutical composition for treating and/or preventing an oncological or hyperproliferative disease, in particular cancer (such as e.g. a cancer disease as described herein), in combination with an anti-IGF antibody as described herein.

In another aspect, the invention refers to the use of an anti-IGF antibody as described herein for preparing a pharmaceutical composition for treating and/or preventing an oncological or hyperproliferative disease, in particular cancer (such as e.g. a cancer disease as described herein), in combination with a dual Aurora kinase/MEK inhibitor as described herein.

In another aspect, the invention refers to the use of a dual Aurora kinase/MEK inhibitor and an anti-IGF antibody, each as described herein, for preparing a pharmaceutical composition for treating and/or preventing an oncological or hyperproliferative disease, in particular cancer (such as e.g. a cancer disease as described herein).

In another aspect, the invention refers to a combination, composition or kit according to the invention comprising, consisting or consisting essentially of a dual Aurora kinase/MEK inhibitor and an anti-IGF antibody, each as described herein, e.g. for treating and/or preventing an oncological or hyperproliferative disease, in particular cancer (e.g. a cancer disease as described herein), optionally in combination with one or more other therapeutic agents.

In another aspect, the invention refers to a combination or kit comprising
a.) a dual Aurora kinase/MEK inhibitor and optionally one or more pharmaceutically acceptable carriers, excipients and/or vehicles,
b.) an anti-IGF antibody and optionally one or more pharmaceutically acceptable carriers, excipients and/or vehicles,
and optionally a package insert comprising printed instructions for simultaneous, concurrent, sequential, successive, alternate or separate use in the treatment and/or prevention of an oncological or hyperproliferative disease, in particular cancer, optionally in combination with one or more other therapeutic agents, in a patient in need thereof.

In another aspect, the invention refers to a combination, composition or kit according to the invention optionally further comprising one or more other therapeutic agents.

In another aspect, the invention refers to a method or use according to the invention optionally further comprising administering or involving one or more other therapeutic agents.

### BRIEF DESCRIPTION OF THE FIGURES

FIGS. 1A-1G show an ELISA binding titration of IgG1 antibodies designated 60814, 60819 and 60833 to human IGF-1 (FIG. 1A), mousee IGF-1 (FIG. 1B), rat IGF-1 (FIG. 1C), human IGF-2 (FIG. 1D), mouse IGF-2 (FIG. 1E), rat IGF-2 (FIG. 1F), and human insulin (FIG. 1G).

FIG. 2 shows typical titrations of antibody 60833 neutralising IGF-1 (20 ng/mL)(FIG. 2A) and IGF-2 (100 ng/mL)(FIG. 2B) induced phosphorylation of the IGF-1R using a cell based ELISA.

FIG. 3A shows a typical titration of antibody 60833 neutralising IGF-2 (100 ng/mL) induced IR-A phosphorylation. FIG. 3B shows a typical titration of antibody 60833 neutralising human serum (20%) induced phosphorylation of the IGF-1R. Both assays are performed using cell based ELISAs.

FIGS. 4A-2D show the effect of antibodies 60814 and 60819 on IGF-1 (FIGS. 4A and 4C) and IGF-2 (FIGS. 4B and 4D) stimulated MCF-7 (FIGS. 4A and 4B) and COLO 205 (FIGS. 4C and 4D) cell proliferation.

FIG. 5 shows the effect of antibodies 60819 and 60833 on the proliferation of the Ewing's sarcoma-derived cell line TC-71 in 10% growth medium.

FIG. 6 shows the effect of antibody 60819 on murine total serum IGF-1 levels 24 hours following the administration of single doses of 25, 12.5, 6.25, 3.13 mg/kg. 0 mg/kg represents the total serum IGF-1 levels prior to antibody treatment.

FIG. 7 shows the effect of antibody 60819 on rat total plasma IGF-1 levels 24 hours following the administration of single doses of 30, 100, 200 mg/kg by a 10 minute intravenous infusion. 0 mg/kg represents the total serum IGF-1 levels prior to antibody treatment.

FIG. 8 demonstrates the effect of antibody 60819 and rapamycin, alone or in combination, on the proliferation of the Ewing's sarcoma-derived cell line SK-ES-1 in 10% FCS containing growth medium.

FIG. 9 shows the effect of antibody 60819 and rapamycin, alone or in combination, on the phosphorylation of AKT and levels of PTEN.

FIG. 10 demonstrates the effect of antibody 60819 and erlotinib/Tarceva, alone or in combination, on the proliferation of the NSCLC-derived cell line A-549 in 10% FCS containing growth medium.

FIG. 11 shows the 3D structure of human IGF-1 where the amino acids bound by antibody 60833 are highlighted (dark grey). The linear amino acid sequence of human IGF-1 where the amino acids that interact with antibody 60833 are underlined is shown underneath.

FIG. 12 shows the amino acid and DNA sequences of the variable chains of antibodies 60814 (A), 60819 (B), and 60833 (C); CDRs are in bold letters.

FIGS. 13 and 14 show values of relative cell growth inhibition (CGI %) and excess inhibition over the predicted Bliss additivism for rating of combination effects obtained by using the dual MEK/AKI #6+anti-IGF mAb 60833 of the present invention (embodiment C1.6 of Table i) in cell lines NCl-H460 and Calu-6, respectively.

### DUAL AURORA KINASE/MEK INHIBITOR

A dual Aurora kinase/MEK inhibitor within the meaning of this invention refers to a compound which is both an inhibitor of one or more Aurora kinases (particularly of Aurora-B) and an inhibitor of one or more MEK kinases (MEK1 and/or MEK2). For the avoidance of any doubt, a dual Aurora kinase/MEK inhibitor within the meaning of this invention refers to one compound having said two different properties, namely that of an Aurora kinase inhibitor (AKI) and that of a MEK inhibitor.

Aurora kinases (Aurora-A, Aurora-B, Aurora-C) are serine/threonine protein kinases that are essential for proliferating cells and have been identified as key regulators of different steps in mitosis and meiosis, ranging from the formation of the mitotic spindle to cytokinesis. Aurora family kinases are critical for cell division, and have beeen closely linked to tumorigenesis and cancer susceptibility. In various human cancers over-expression and/or up-regulation of kinase activity of Aurora-A, Aurora-B and/or Aurora C has been observed. Over-expression of Aurora kinases correlates clinically with cancer progression and poor survival prognosis. Aurora kinases are involved in phosphorylation events (e.g. phosphorylation of histone H3) that regulate the cell cycle. Misregulation of the cell cycle can lead to cellular proliferation and other abnormalities.

The serine/threonine kinase Aurora-B is involved in the regulation of several mitotic processes, including chromosome condensation, congression and segregation as well as cytokinesis. Inactivation of Aurora B abrogates the spindle assembly checkpoint (SAC) and causes premature mitotic exit without cytokinesis, resulting in polyploid cells that eventually stop further DNA replication. Aurora B inhibitors induce a mitotic override (mitotic slippage). Inhibitors of Aurora B kinase also block proliferation in various human cancer cell lines and induce polyploidy, senescence and apoptosis.

Aurora B inhibitors abrogate the spindle assembly checkpoint (SAC) and induce a mitotic override (mitotic slippage), yielding aberrant polyploid cells rather then a cell cycle arrest. Polyploid cells spend little time in mitosis as check point controls are overridden and become genetically unstable. Inhibition of Aurora B kinase can predominantly induce slow senescence-associated cell death rather than apoptosis which may distinguish it from other anti-mitotic principles. In common with other M-phase targeting drugs is the general applicablility of this anti-cancer treatment principle. Aurora kinases are indeed restrictedly expressed during mitosis and thus exclusively found in proliferating cells.

MEK (mitogen-activated protein kinase/extracellular signal related kinase kinase) is a key player in the “RAS-RAF-MEK-ERK pathway” which has pathophysiological relevance in various cancer types. The direct downstream substrate of MEK is ERK which in its phosphorylated state enters the cell nucleus and is involved in the regulation of gene expression. MEK is frequently activitated in tumors, especially when either RAS or BRAF is mutated. BRAF and RAS mutations are known to be mutually exclusive. According to the literature, RAF-inhibitors are not active in KRAS mutated cancers, whereas MEK inhibitors could principally work in both KRAS and BRAF mutated cancers (see also Table 1 below). No difference in relevance and function between the two MEK isoforms (MEK1, MEK2) is known to date. The RAS-dependent RAF/MEK/ERK1/2 mitogen activated protein (MAP) kinase signaling pathway plays an important role in the regulation of cell proliferation and survival.

Constitutive activation of the RAS/RAF/MEK/ERK signaling pathway is involved in malignant transformation. Mutational activation of KRAS (approximately 15% of all cancers) and BRAF (about 7% of all cancers) are common mutually exclusive events found in a variety of human tumors (see Table 1 below).

TABLE 1
Occurrence of BRAF and RAS mutations in various cancers

KRAS mutation:
~70% Pancreas
~37% CRC
~18% NSCLC
~14% Ovarian
~8% Prostate
~5% Breast
~4% HCC
NRAS mutation:
~20% Melanoma
BRAF mutation:
~46% Thyroid
~43% Melanoma
~12% Ovarian
~11% CRC
~7% Prostate
<5% NSCLC
CRC: Colorectal cancer
NSCLC: Non-small cell lung cancer
HCC: Hepatocellular cancer

Taken together, a dual Aurora kinase/MEK inhibitor of this invention—as an inhibitor of Aurora B kinase, a target essential for mitosis of all cancer cells independent of oncogenic mutations—shows efficacy in a broad range of cancers by inducing polyploidy and senescence. In addition, due to potent inhibition of MEK signaling, a dual Aurora kinase/MEK inhibitor of this invention is particularly effective in a subset of cancers dependent on oncogenic MEK signaling due to mutations in RAS or RAF genes.

In a particular embodiment of this invention, a dual Aurora kinase/MEK inhibitor according to this invention is both an inhibitor of Aurora kinase B and an inhibitor of the kinases MEK1 and/or MEK2.

Examples of dual Aurora kinase/MEK inhibitors according to this invention can be found in WO 2010/012747, the disclosure of which is incorporated herein by reference in its entirety.

Preferably, a dual Aurora kinase/MEK inhibitor within this invention is selected from the Group X consisting of the following compounds 1 to 25, optionally in the form of the tautomers or pharmaceutically acceptable salts thereof:

### Group X:

### 1) N-ethyl-3-[3-[[4-(4-methylpiperazin-1-yl)anilino]-phenylmethylidene]-2-oxo-1H-indol-6-yl]prop-2-ynamide

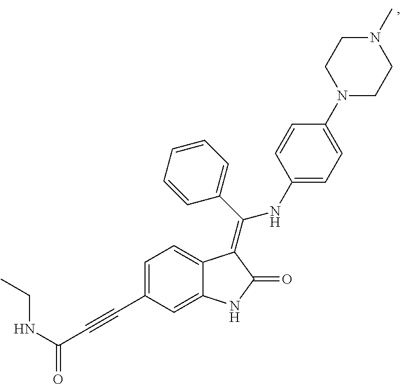 [SMILES: CCNC(=O)C#Cc1ccc2c(c1)NC(=O)/C2=C(\Nc1ccc(N2CCN(C)CC2)cc1)c1ccccc1]

### 2) N-(2,2-difluoroethyl)-3-[3-[[4-(dimethylaminomethyl)anilino]-phenylmethylidene]-2-oxo-1H-indol-6-yl]prop-2-ynamide

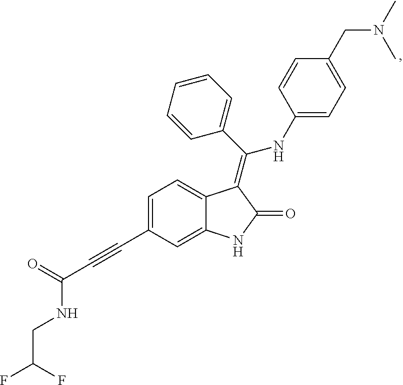 [SMILES: CN(C)Cc1ccc(N/C(=C2\C(=O)Nc3cc(C#CC(=O)NCC(F)F)ccc32)c2ccccc2)cc1]

### 3) N-(2,2-difluoroethyl)-3-[2-oxo-3-[phenyl-[4-(pyrrolidin-1-ylmethyl)anilino]methylidene]-1H-indol-6-yl]prop-2-ynamide

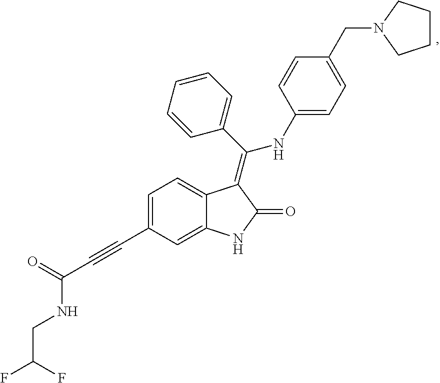 [SMILES: O=C(C#Cc1ccc2c(c1)NC(=O)/C2=C(\Nc1ccc(CN2CCCC2)cc1)c1ccccc1)NCC(F)F]

### 4) N-(2-fluoroethyl)-3-[2-oxo-3-[phenyl-[4-(pyrrolidin-1-ylmethyl)anilino]methylidene]-1H-indol-6-yl]prop-2-ynamide

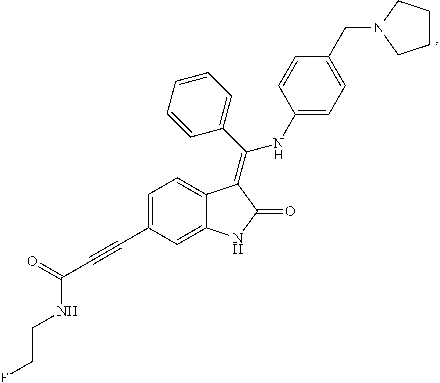 [SMILES: O=C(C#Cc1ccc2c(c1)NC(=O)/C2=C(\Nc1ccc(CN2CCCC2)cc1)c1ccccc1)NCCF]

### 5) N-ethyl-3-[2-oxo-3-[phenyl-[4-(pyrrolidin-1-ylmethyl)anilino]methylidene]-1H-indol-6-yl]prop-2-ynamide

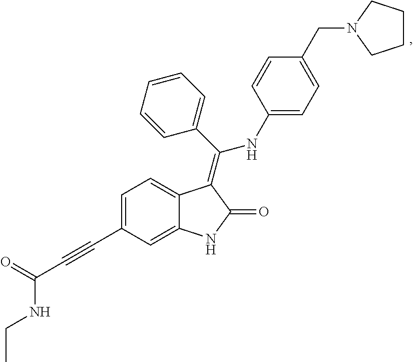 [SMILES: CCNC(=O)C#Cc1ccc2c(c1)NC(=O)/C2=C(\Nc1ccc(CN2CCCC2)cc1)c1ccccc1]

### 6) 3-[3-[[4-(dimethylaminomethyl)anilino]-phenylmethylidene]-2-oxo-1H-indol-6-yl]-N-ethylprop-2-ynamide

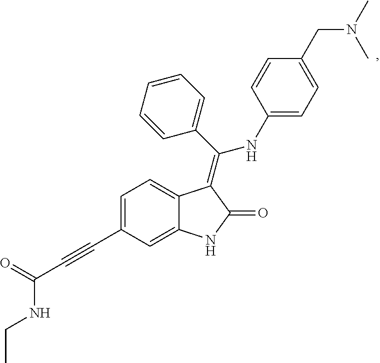 [SMILES: CCNC(=O)C#Cc1ccc2c(c1)NC(=O)/C2=C(\Nc1ccc(CN(C)C)cc1)c1ccccc1]

### 7) N-cyclobutyl-3-[3-[[4-(4-methylpiperazin-1-yl)anilino]-phenylmethylidene]-2-oxo-1H-indol-6-yl]prop-2-ynamide

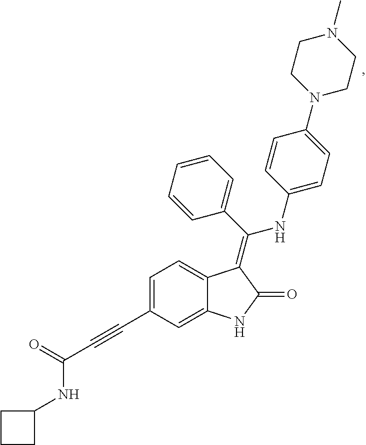 [SMILES: CN1CCN(c2ccc(N/C(=C3\C(=O)Nc4cc(C#CC(=O)NC5CCC5)ccc43)c3ccccc3)cc2)CC1]

### 8) N-cyclopropyl-3-[3-[[4-(dimethylaminomethyl)anilino]-phenylmethylidene]-2-oxo-1H-indol-6-yl]prop-2-ynamide

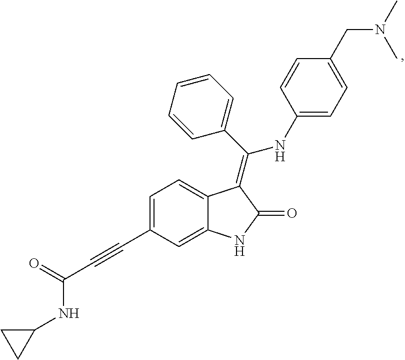 [SMILES: CN(C)Cc1ccc(N/C(=C2\C(=O)Nc3cc(C#CC(=O)NC4CC4)ccc32)c2ccccc2)cc1]

### 9) 3-[3-[[4-(dimethylaminomethyl)anilino]-phenylmethylidene]-2-oxo-1H-indol-6-yl]-N-phenylprop-2-ynamide

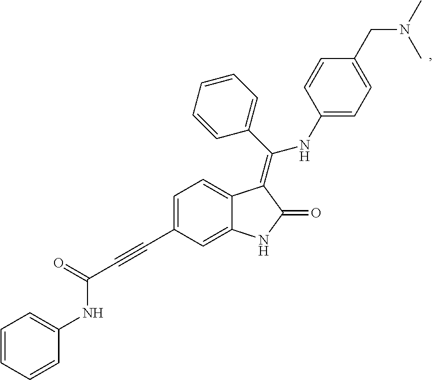 [SMILES: CN(C)Cc1ccc(N/C(=C2\C(=O)Nc3cc(C#CC(=O)Nc4ccccc4)ccc32)c2ccccc2)cc1]

### 10) N-cyclopentyl-3-[3-[[4-(dimethylaminomethyl)anilino]-phenylmethylidene]-2-oxo-1H-indol-6-yl]prop-2-ynamide

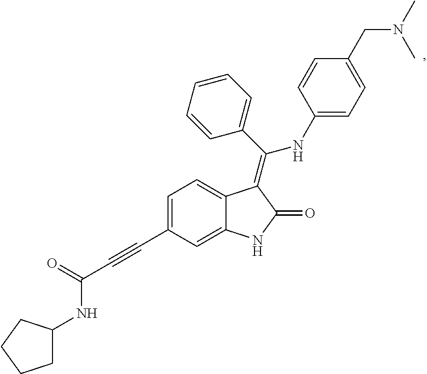 [SMILES: CN(C)Cc1ccc(N/C(=C2\C(=O)Nc3cc(C#CC(=O)NC4CCCC4)ccc32)c2ccccc2)cc1]

### 11) N-cyclopentyl-3-[3-[[4-(4-methylpiperazin-1-yl)anilino]-phenylmethylidene]-2-oxo-1H-indol-6-yl]prop-2-ynamide

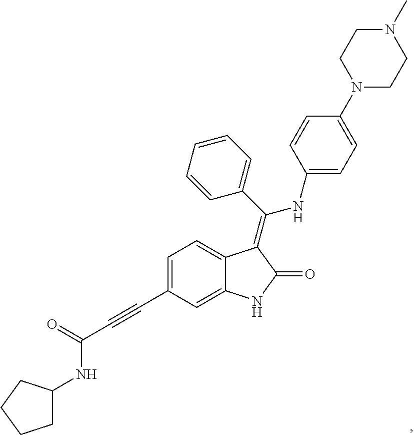 [SMILES: CN1CCN(c2ccc(N/C(=C3\C(=O)Nc4cc(C#CC(=O)NC5CCCC5)ccc43)c3ccccc3)cc2)CC1]

### 12) N-cyclobutyl-3-[3-[[4-(dimethylaminomethyl)anilino]-phenylmethylidene]-2-oxo-1H-indol-6-yl]prop-2-ynamide

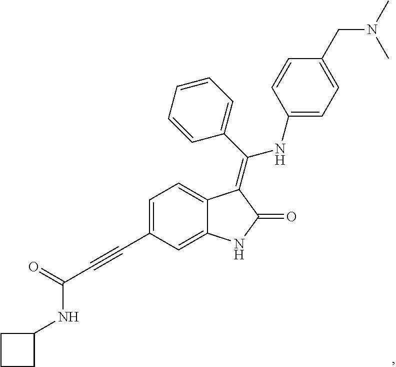 [SMILES: CN(C)Cc1ccc(N/C(=C2\C(=O)Nc3cc(C#CC(=O)NC4CCC4)ccc32)c2ccccc2)cc1]

### 13) 3-[3-[[4-(dimethylaminomethyl)anilino]-phenylmethylidene]-2-oxo-1H-indol-6-yl]-N-(2-hydroxyethyl)prop-2-ynamide

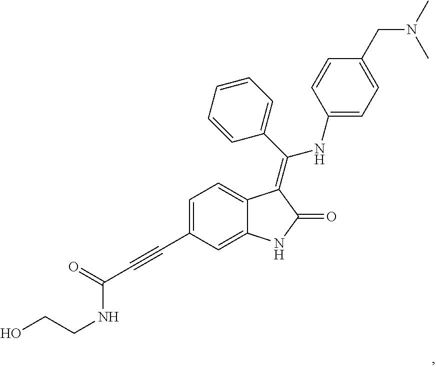 [SMILES: CN(C)Cc1ccc(N/C(=C2\C(=O)Nc3cc(C#CC(=O)NCCO)ccc32)c2ccccc2)cc1]

### 14) 3-[3-[[4-(dimethylaminomethyl)anilino]-phenylmethylidene]-2-oxo-1H-indol-6-yl]-N-propan-2-ylprop-2-ynamide

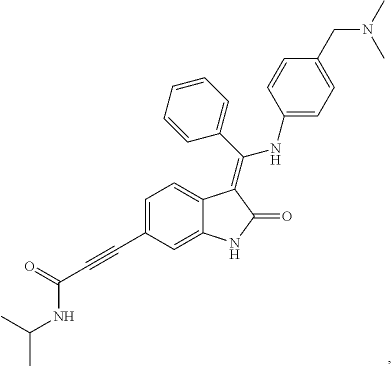 [SMILES: CC(C)NC(=O)C#Cc1ccc2c(c1)NC(=O)/C2=C(\Nc1ccc(CN(C)C)cc1)c1ccccc1]

### 15) 3-[2-oxo-3-[phenyl-[4-(pyrrolidin-1-ylmethyl)anilino]methylidene]-1H-indol-6-yl]-N-propan-2-ylprop-2-ynamide

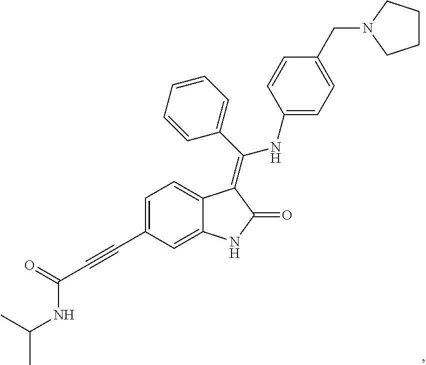 [SMILES: CC(C)NC(=O)C#Cc1ccc2c(c1)NC(=O)/C2=C(\Nc1ccc(CN2CCCC2)cc1)c1ccccc1]

### 16) N-(2-hydroxyethyl)-3-[2-oxo-3-[phenyl-[4-(pyrrolidin-1-ylmethyl)anilino]methylidene]-1H-indol-6-yl]prop-2-ynamide

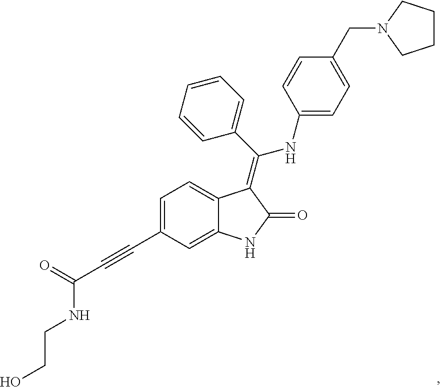 [SMILES: O=C(C#Cc1ccc2c(c1)NC(=O)/C2=C(\Nc1ccc(CN2CCCC2)cc1)c1ccccc1)NCCO]

### 17) N-(2-fluorophenyl)-3-[2-oxo-3-[phenyl-[4-(pyrrolidin-1-ylmethyl)anilino]methylidene]-1H-indol-6-yl]prop-2-ynamide

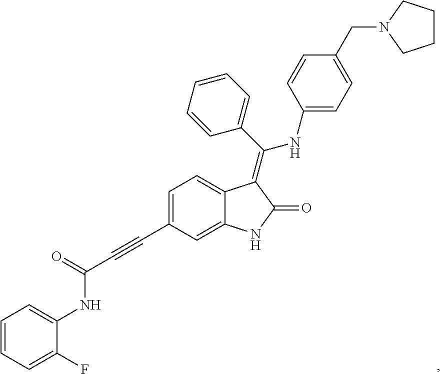 [SMILES: O=C(C#Cc1ccc2c(c1)NC(=O)/C2=C(\Nc1ccc(CN2CCCC2)cc1)c1ccccc1)Nc1ccccc1F]

### 18) 3-[3-[[4-(dimethylaminomethyl)anilino]-phenylmethylidene]-2-oxo-1H-indol-6-yl]-N-[(2S)-1-hydroxypropan-2-yl]prop-2-ynamide

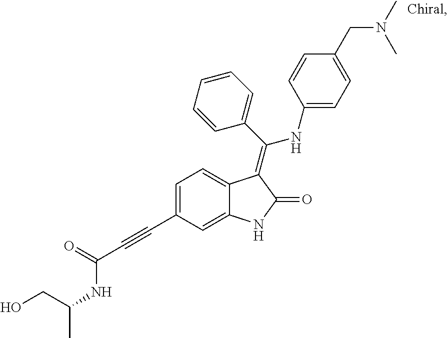 [SMILES: C[C@H](CO)NC(=O)C#Cc1ccc2c(c1)NC(=O)/C2=C(\Nc1ccc(CN(C)C)cc1)c1ccccc1]

### 19) N-[(2S)-1-hydroxypropan-2-yl]-3-[2-oxo-3-[phenyl-[4-(pyrrolidin-1-ylmethyl)anilino]methylidene]-1H-indol-6-yl]prop-2-ynamide

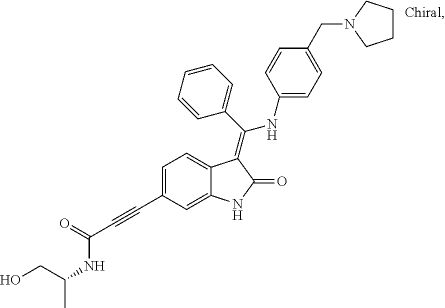 [SMILES: C[C@H](CO)NC(=O)C#Cc1ccc2c(c1)NC(=O)/C2=C(\Nc1ccc(CN2CCCC2)cc1)c1ccccc1]

### 20) N-[(2R)-butan-2-yl]-3-[2-oxo-3-[phenyl-[4-(pyrrolidin-1-ylmethyl)anilino]methylidene]-1H-indol-6-yl]prop-2-ynamide

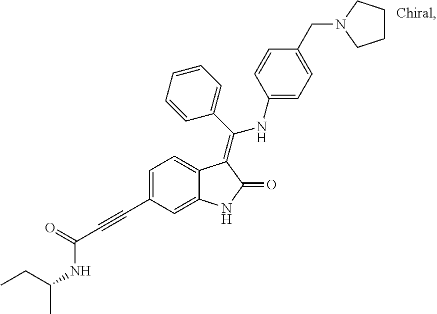 [SMILES: CC[C@@H](C)NC(=O)C#Cc1ccc2c(c1)NC(=O)/C2=C(\Nc1ccc(CN2CCCC2)cc1)c1ccccc1]

### 21) N-(3-chlorophenyl)-3-[2-oxo-3-[phenyl-[4-(pyrrolidin-1-ylmethyl)anilino]methylidene]-1H-indol-6-yl]prop-2-ynamide

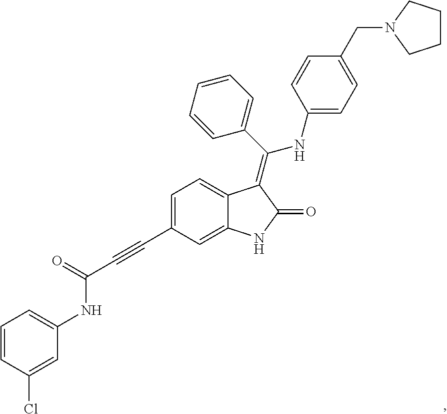 [SMILES: O=C(C#Cc1ccc2c(c1)NC(=O)/C2=C(\Nc1ccc(CN2CCCC2)cc1)c1ccccc1)Nc1cccc(Cl)c1]

### 22) N-(3-chlorophenyl)-3-[3-[[4-(dimethylaminomethyl)anilino]-phenylmethylidene]-2-oxo-1H-indol-6-yl]prop-2-ynamide

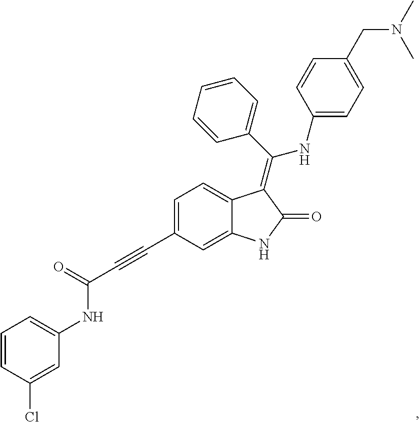 [SMILES: CN(C)Cc1ccc(N/C(=C2\C(=O)Nc3cc(C#CC(=O)Nc4cccc(Cl)c4)ccc32)c2ccccc2)cc1]

### 23) 3-[2-oxo-3-[phenyl-[4-(pyrrolidin-1-ylmethyl)anilino]methylidene]-1H-indol-6-yl]-N-phenylprop-2-ynamide

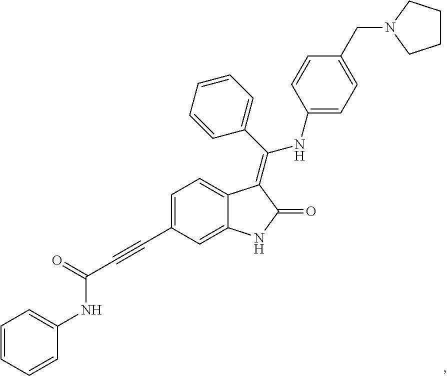 [SMILES: O=C(C#Cc1ccc2c(c1)NC(=O)/C2=C(\Nc1ccc(CN2CCCC2)cc1)c1ccccc1)Nc1ccccc1]

### 24) 3-[2-oxo-3-[phenyl-[4-(pyrrolidin-1-ylmethyl)anilino]methylidene]-1H-indol-6-yl]-N-pentan-3-ylprop-2-ynamide

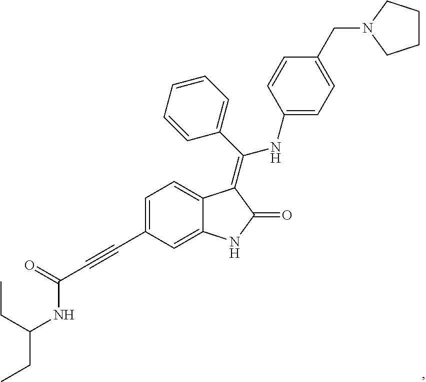 [SMILES: CCC(CC)NC(=O)C#Cc1ccc2c(c1)NC(=O)/C2=C(\Nc1ccc(CN2CCCC2)cc1)c1ccccc1]

and

### 25) N-(3-fluorophenyl)-3-[2-oxo-3-[phenyl-[4-(pyrrolidin-1-ylmethyl)anilino]methylidene]-1H-indol-6-yl]prop-2-ynamide

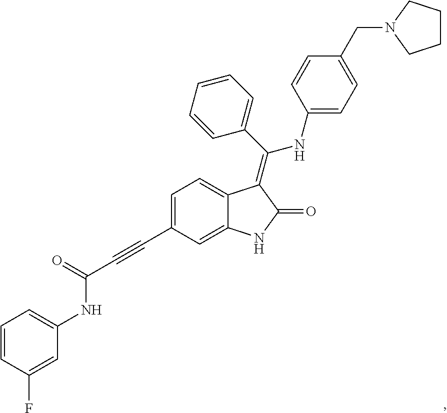 [SMILES: O=C(C#Cc1ccc2c(c1)NC(=O)/C2=C(\Nc1ccc(CN2CCCC2)cc1)c1ccccc1)Nc1cccc(F)c1]

The dual inhibitory activity of the AKI/MEK inhibitors (or also named dual MEK/AKI) according to this invention can be determined according to methods customary to the skilled person, e.g. by methods known in the literature or as described herein or analogously thereto. Assays for measuring the Aurora kinase inhibitory activity as well as assays for measuring the MEK inhibitory activity of a compound are known from literature, are commercially available or are described herein in the examples section.

As stated herein, a dual Aurora kinase/MEK inhibitor in the scope of the present invention relates to a compound that exhibits inhibitory activity both on an Aurora kinase and on a kinase of MEK. Such inhibitory activity can be characterised each by the IC50 value.

A dual Aurora kinase/MEK inhibitor of this invention has preferably an IC50 value for inhibition of an Aurora kinase (particularly Aurora B kinase) below 200 nM, preferably below 40 nM, more preferably below 10 nM (e.g. from about 1 nM to about 10 nM), preferably measured in the assay given in the following examples.

A dual Aurora kinase/MEK inhibitor of this invention has preferably an IC50 value for inhibition of a MEK kinase (MEK1 and/or MEK2) below 1000 nM, preferably below 200 nM, more preferably below 100 nM, even more preferably below 50 nM (e.g. below 30 nM), preferably measured in the assay given in the following examples.

A dual Aurora kinase/MEK inhibitor of this invention may have, for example, an IC50 value for inhibition of Aurora B kinase below 200 nM, preferably below 40 nM, more preferably below 10 nM (e.g. from about 1 nM to about 10 nM), and an IC50 value for inhibition of a MEK kinase (MEK1 and/or MEK2) below 1000 nM, preferably below 200 nM, more preferably below 100 nM, even more preferably below 50 nM (e.g. from about 1 nM to about 50 nM, such as e.g. MEK1 IC50 from about 1 nM to about 25 nM), preferably measured in the assays given in the following examples.

For illustrative example, the dual Aurora kinase/MEK inhibitors 1 to 6 of Group X indicated above have IC50 values for inhibition of Aurora kinase B from about 2 nM to about 7 nM and IC50 values for inhibition of MEK1 from about 3 nM to about 25 nM (see table as follows), measured in the assays given in the examples section:

| | Aurora B | MEK 1 |
|---|---|---|
| Compound No. | IC50 [nM] | IC50 [nM] |
| 1 | 2 | 10 |
| 2 | 7 | 6 |
| 3 | 4 | 3 |
| 4 | 5 | 6 |
| 5 | 5 | 5 |
| 6 | 3 | 25 |

This dual activity can also be confirmed in respective biomarker assays, such as e.g. in a phospho-histone H3 assay (e.g. H460, Cellomics), where p-histone H3 as marker for Aurora B kinase inhibition is inhibited, and in a phospho-ERK assay (e.g. SK-MEL 28, FACE ELISA), where p-ERK as marker for MEK inhibition is inhibited.

For example, a dual Aurora kinase/MEK inhibitor of this invention may have an EC50 value for reduction of phospho-histone H3 below 1000 nM, preferably below 200 nM, more preferably below 100 nM (e.g. from about 10 nM to about 50 nM), and an EC50 value for reduction of phospho-ERK below 1000 nM, preferably below 200 nM, more preferably below 100 nM (e.g. from about 30 nM to about 70 nM), preferably measured in the assays given in the following examples.

A certain exemplary dual Aurora kinase/MEK inhibitor of Group X of this invention has IC50 value for inhibition of Aurora kinase B of 3 nM and IC50 values for inhibition of MEK1 and MEK2 of 25 nM and 4 nM, respectively, and has EC50 for reduction of phospho-histone H3 of 44 nM (synchronized H460 cells, 1 h treatment, molecular phosphorylation assay, Cellomics) and EC50 for reduction of phospho-ERK of 59 nM (SK-MEL 28 melanoma cells, FACE ELISA), measured in the assays given in the examples section.

Direct inhibition of the MAP-kinase signaling pathway by the dual Aurora kinase/MEK inhibitors of this invention can be further confirmed in A375 and BRO melanoma cells.

The inhibitory activity on Aurora B kinase can be further confirmed by polyploidy phenotype. A certain exemplary dual Aurora kinase/MEK inhibitor of Group X of this invention induces polyploidy in H460 cells as determined by DNA content analyses (Cellomics ArrayScan) over a wide range of concentrations. At 7 nM, 81% of the cells are polyploid after a 42 h exposure to the compound.

The cellular potency can be determined in various assays including Alamar Blue based proliferation assays performed in the presence of 10% fetal calf serum. For example, a dual Aurora kinase/MEK inhibitor of this invention may have an EC50 value in cell based proliferation assay below 1000 nM, preferably below 200 nM, more preferably below 100 nM, even more preferably below 50 nM (e.g. from about 5 nM to about 20 nM). A certain exemplary dual Aurora kinase/MEK inhibitor of Group X of this invention inhibits the proliferation of 5 tumour cell lines tested (see table as follows):

| Cell line | Origin | EC₅₀ [nM] |
|---|---|---|
| NCI-H460 | NSCLC | 8 |
| A549 | NSCLC | 7 |
| HCT 116 | Colorectal carcinoma | 10 |
| A375 | Melanoma | 5 |
| PC-3 | Prostate carcinoma | 6 |

Many of the cell lines which are sensitive to a dual Aurora kinase/MEK inhibitor of this invention are mutated either in the RAS or the RAF genes.

The term dual Aurora kinase/MEK inhibitor as used herein also comprises any tautomers, pharmaceutically acceptable salts thereof, hydrates and solvates thereof, including the respective crystalline forms.

The dual Aurora kinase/MEK inhibitor compounds 1 to 25 of Group X can be synthesized as described in WO 2010/012747 or analogously or similarly thereto, e.g. as shown in the following reaction scheme, where R1 and R have the meanings as defined in the compounds 1 to 25 and L denotes a suitable leaving group, such as e.g. bromine or iodine. The indolinone intermediate compounds are known or they can be synthesized using standard methods of synthesis or analogously to the methods described in WO 2007/122219 or WO 2008/152013 or as shown by way of example in the following reaction scheme. The propynoic acid amides are known or can be prepared according to standard methods.

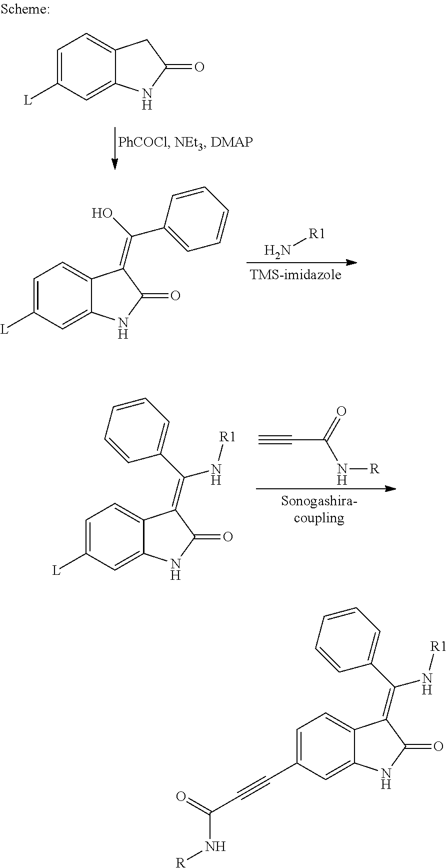

It is moreover known to the person skilled in the art that if there are a number of reactive centers on a starting or intermediate compound it may be necessary to block one or more reactive centers temporarily by protective groups in order to allow a reaction to proceed specifically at the desired reaction center. After the desired reaction has occurred, the protective group is usually removed in a suitable manner. A detailed description for the use of a large number of proven protective groups is found, for example, in “Protective Groups in Organic Synthesis” by T. Greene and P. Wuts (John Wiley & Sons, Inc. 2007, 4th Ed.) or in “Protecting Groups (Thieme Foundations Organic Chemistry Series N Group” by P. Kocienski (Thieme Medical Publishers, 2004).

In a further embodiment, within the present invention it is referred to a dual Aurora kinase/MEK inhibitor as described herein for use in medicine.

In a further embodiment, within the present invention it is referred to a pharmaceutical composition containing, as the active ingredient, a dual Aurora kinase/MEK inhibitor of the invention.

To be used in therapy, the dual Aurora kinase/MEK inhibitor, optionally in combination with one or more other active agents, is included into pharmaceutical compositions appropriate to facilitate administration to animals or humans.

Typical pharmaceutical compositions for administering the dual Aurora kinase/MEK inhibitor of the invention include for example tablets, capsules, suppositories, solutions,—e.g. solutions for injection (s.c., i.v., i.m.) and infusion—elixirs, emulsions or dispersible powders. The content of the pharmaceutically active compound(s) may be in the range from 0.1 to 90 wt.-%, preferably 0.5 to 50 wt.-% of the composition as a whole, e.g. in amounts which are sufficient to achieve the desired dosage range. The single dosages may, if necessary, be given several times a day to deliver the desired total daily dose.

Typical tablets may be obtained, for example, by mixing the active substance(s), optionally in combination, with known excipients, for example inert diluents such as calcium carbonate, calcium phosphate, cellulose or lactose, disintegrants such as corn starch or alginic acid or crospovidon, binders such as starch or gelatine, lubricants such as magnesium stearate or talc and/or agents for delaying release, such as carboxymethyl cellulose, cellulose acetate phthalate, or polyvinyl acetate. The tablets may be prepared by usual processes, such as e.g. by direct compression or roller compaction. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substance(s) may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing the active substance(s) may for example be prepared by mixing the active substance(s) with inert carriers such as lactose or sorbitol and packing them into gelatine capsules.

Typical suppositories may be made for example by mixing the active substance(s) with carriers provided for this purpose, such as neutral fats or polyethyleneglycol or the derivatives thereof. Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The dual Aurora kinase/MEK inhibitors of this invention are administered by the usual methods, preferably by oral or parenteral route, most preferably by oral route. For oral administration the tablets may contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for oral use is from 1-2000 mg per day (e.g. from 50 to 700 mg per day). The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

### Anti-IGF Antibody

An anti-IGF antibody within the meaning of this invention refers to an anti-IGF antibody molecule, which binds preferably to human IGF-1 and/or IGF-2.

Insulin-like growth factor-1 (IGF-1; a 70 amino-acid polypeptide) and insulin-like growth factor-2 (IGF-2; a 67 amino-acid polypeptide) are 7.5-kD soluble factors present in serum that can potently stimulate the growth of many mammalian cells (reviewed by Pollack et al., 2004). On secretion into the bloodstream the IGFs form complexes with the IGFBPs which protect them from proteolytic degradation in the serum en route to their target tissues and prevents their association with the IGF receptors. IGFs are also known to be secreted in an autocrine or paracrine manner in target tissues themselves. This is known to occur during normal fetal development where the IGFs play a key role in the growth of tissues, bone and organs. It is also seen in many cancer tissues where there is thought to be paracrine signaling between tumour cells and stromal cells or autocrine IGF production by the tumour cells themselves (reviewed by LeRoith D, 2003).

IGF-1 and IGF-2 are able to bind to the IGF-1 receptor (IGF-1R) expressed on many normal tissues, which functionally is a 460 kD heterotetramer consisting of a dimerised alpha- and beta-subunit, with similar affinities (Rubin et al., 1995). IGF-2 can also bind to the IGF-2 receptor, which is thought to prevent IGF-2 from binding and signaling through the IGF-1R. In this respect the IGF-2R has been demonstrated to be a tumour suppressor protein. The IGF-1R is structurally similar to the insulin receptor which exists in two forms, IR-A and IR-B, which differ by an alternatively spliced 12 amino acid exon deletion in the extracellular domain of IR-A. IR-B is the predominant IR isoform expressed in most normal adult tissues where it acts to mediate the effects of insulin on metabolism. IR-A on the other hand is known to be highly expressed in developing fetal tissues but not in adult normal tissues. Recent studies have also shown that IR-A, but not IR-B, is highly expressed in some cancers. The exon deletion in IR-A has no impact on insulin binding but does cause a small conformational change that allows IGF-2 to bind with much higher affinity than for IR-B (Frasca et al., 1999; Pandini et al., 2002). Thus, because of it's expression in cancer tissues and increased propensity for IGF-2 binding, IR-A may be as important as IGF1-R in mediating the mitogenic effects of IGF-2 in cancer.

Binding of the IGFs to IGF-1R triggers a complex intracellular signaling cascade which results in activation of proteins that stimulate proliferation and survival (reviewed by Pollack et al., 2004).

Unlike the EGFR and Her2neu receptors there is no known amplification of the IGF1-R or IR-A receptors in cancers indicating that receptor activation is controlled by the presence of active ligand. There is a very large body of scientific, epidemiological and clinical literature implicating a role for the IGFs in the development, progression and metastasis of many different cancer types (reviewed by Jerome et al., 2003; and Pollack et al., 2004).

In an embodiment, the anti-IGF antibody within this invention is a human anti-IGF antibody with high affinity.

In a further embodiment the anti-IGF antibody within this invention is a human anti-IGF antibody with high affinity to IGF-1.

In a further embodiment the anti-IGF antibody within this invention is a human anti-IGF antibody with high affinity to IGF-1 and to IGF-2.

In a further embodiment the anti-IGF antibody within this invention is a human anti-IGF antibody with adequate relative affinity to IGF-1 and to IGF-2.

In a further embodiment the anti-IGF antibody within this invention is a human anti-IGF antibody with a higher affinity to IGF-1 than to IGF-2.

In a further embodiment the anti-IGF antibody within this invention is a human anti-IGF antibody with high IGF-1 neutralisation potency.

In a further embodiment the anti-IGF antibody within this invention is a human anti-IGF antibody with high IGF-1 and IGF-2 neutralisation potency.

In a further embodiment the anti-IGF antibody within this invention is a human anti-IGF antibody with high solubility and stability.

In a further embodiment the anti-IGF antibody within this invention is a human anti-IGF antibody that does not affect binding of insulin to its receptor.

The term “anti-IGF antibody molecule” encompasses human anti-IGF antibodies, anti-IGF antibody fragments, anti-IGF antibody-like molecules and conjugates with any of the herein mentioned antibody molecules. Antibodies include, in the meaning of the present invention, but are not limited to, monoclonal, chimerized monoclonal, and bi- or multispecific antibodies. The term “antibody” shall encompass complete immunoglobulins as they are produced by lymphocytes and for example present in blood sera, monoclonal antibodies secreted by hybridoma cell lines, polypeptides produced by recombinant expression in host cells, which have the binding specificity of immunoglobulins or monoclonal antibodies, and molecules which have been derived from such immunoglobulins, monoclonal antibodies, or polypeptides by further processing while retaining their binding specificity.

In particular, the term “antibody molecule” includes fully human complete immunoglobulins comprising two heavy chains and two light chains, preferably.

In a further embodiment, the antibody molecule is an anti-IGF antibody-fragment that has an antigen binding region. To obtain antibody fragments, e.g. Fab fragments, digestion can be accomplished by means of routine techniques, e.g. using papain or pepsin. Examples of papain digestion are described in WO 94/29348 and U.S. Pat. No. 4,342,566. Papain digestion of antibodies typically produces two identical antigen binding fragments, so-called Fab fragments, each with a single antigen binding site, and a residual Fc fragment. Pepsin treatment yields an F(ab′)₂ fragment that has two antigen combining sites and is still capable of cross-linking the antigen. Antibody fragments can also be generated by molecular biology methods producing the respective coding DNA fragments.

Fab fragments also contain the constant domains of the light chain and the first constant domain (CH₁) of the heavy chain. Fab′ fragments differ from Fab fragments in that they contain additional residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them.

Antigen-binding antibody fragments or antibody-like molecules, including single-chain antibodies and linear antibodies as described in Zapata et al., 1995, may comprise, on a single polypeptide, the variable region(s) alone or in combination with the entirety or a portion of the following: constant domain of the light chain, CH1, hinge region, CH2, and CH3 domains, e.g. a so-called “SMIP” (“Small Modular Immunopharmaceutical”), which is an antibody like molecule employing a single polypeptide chain as its binding domain Fv, which is linked to single-chain hinge and effector domains devoid of the constant domain CH1 (WO 02/056910). SMIPs can be prepared as monomers or dimers, but they do not assume the dimer-of-dimers structure of traditional antibodies. Also included in the invention are antigen-binding fragments comprising any combination of variable region(s) with a constant domain region of a light chain, VH1, CH1, hinge region, CH2, and CH3 domains.

The antibody fragments or antibody-like molecules may contain all or only a portion of the constant region as long as they exhibit specific binding to the relevant portion of the IGF-1/IGF-2 antigen. The choice of the type and length of the constant region depends, if no effector functions like complement fixation or antibody dependent cellular toxicity are desired, mainly on the desired pharmacological properties of the antibody protein. The antibody molecule will typically be a tetramer consisting of two light chain/heavy chain pairs, but may also be dimeric, i.e. consisting of a light chain/heavy chain pair, e.g. a Fab or Fv fragment, or it may be a monomeric single chain antibody (scFv).

The anti-IGF antibody-like molecules may also be single domain antibodies (e.g. the so-called “nanobodies”), which harbour an antigen-binding site in a single Ig-like domain (described e.g. in WO 03/050531, and by Revets et al., 2005). Other examples for antibody-like molecules are immunoglobulin super family antibodies (IgSF; Srinivasan and Roeske, 2005), or CDR-containing or CDR-grafted molecules or “Domain Antibodies” (dAbs). dABs are functional binding units of antibodies, corresponding to the variable regions of either the heavy (VH) or light (VL) chains of human antibodies. Domain Antibodies have a molecular weight of approximately 13 kDa, or less than one-tenth the size of a full antibody. A series of large and highly functional libraries of fully human VH and VL dAbs has been developed. dABs are also available for “dual targeting”, i.e. dAbs that bind, in addition to IGF-1/IGF-2, to a further target in one molecule. dAb libraries, selection and screening methods, dAb formats for dual targeting and for conferring extended serum half life are described in e.g. U.S. Pat. No. 6,696,245, WO 04/058821, WO 04/003019, and WO 03/002609.

In general, antibody fragments and antibody-like molecules are well expressed in bacterial, yeast, and mammalian cell systems.

Examples of anti-IGF antibodies according to this invention can be found in WO 2010/066868, the disclosure of which is incorporated herein by reference in its entirety.

In a particular embodiment, an anti-IGF antibody within this invention refers to an isolated human antibody molecule, which

a) binds to human IGF-1 and IGF-2 such that
i) binding of IGF-1 and IGF-2 to the IGF-1 receptor is prevented and
ii) IGF-1 receptor-mediated signaling is inhibited,

b) binds to mouse and rat IGF-1 and IGF-2,

c) does not bind to human insulin;

wherein said antibody molecule is selected from the group comprising
i) an antibody molecule that has heavy chain CDRs comprising the amino acid sequences of SEQ ID NO:1 (CDR1), SEQ ID NO:2 (CDR2) and SEQ ID NO:3 (CDR3) and that has light chain CDRs comprising the amino acid sequences of SEQ ID NO:4 (CDR1), SEQ ID NO:5 (CDR2) and SEQ ID NO:6 (CDR3);
ii) an antibody molecule that has heavy chain CDRs comprising the amino acid sequences of SEQ ID NO:11 (CDR1), SEQ ID NO:12 (CDR2) and SEQ ID NO:13 (CDR3) and that has light chain CDRs comprising the amino acid sequences of SEQ ID NO:14 (CDR1), SEQ ID NO:15 (CDR2) and SEQ ID NO:16 (CDR3);
iii) an antibody molecule that has heavy chain CDRs comprising the amino acid sequences of SEQ ID NO:21 (CDR1), SEQ ID NO:22 (CDR2) and SEQ ID NO:23 (CDR3) and that has light chain CDRs comprising the amino acid sequences of SEQ ID NO:24 (CDR1), SEQ ID NO:25 (CDR2) and SEQ ID NO:26 (CDR3).

In another particular embodiment, an anti-IGF antibody within this invention refers to an anti-IGF antibody molecule, wherein said antibody molecule has heavy chain CDRs comprising the amino acid sequences of SEQ ID NO:1 (CDR1), SEQ ID NO:2 (CDR2) and SEQ ID NO:3 (CDR3) and light chain CDRs comprising the amino acid sequences of SEQ ID NO:4 (CDR1), SEQ ID NO:5 (CDR2) and SEQ ID NO:6 (CDR3).

In another particular embodiment, an anti-IGF antibody within this invention refers to an anti-IGF antibody molecule, wherein said antibody molecule has heavy chain CDRs comprising the amino acid sequences of SEQ ID NO:11 (CDR1), SEQ ID NO:12 (CDR2) and SEQ ID NO:13 (CDR3) and light chain CDRs comprising the amino acid sequences of SEQ ID NO:14 (CDR1), SEQ ID NO:15 (CDR2) and SEQ ID NO:16 (CDR3).

In another particular embodiment, an anti-IGF antibody within this invention refers to an anti-IGF antibody molecule, wherein said antibody molecule has heavy chain CDRs comprising the amino acid sequences of SEQ ID NO:21 (CDR1), SEQ ID NO:22 (CDR2) and SEQ ID NO:23 (CDR3) and has light chain CDRs comprising the amino acid sequences of SEQ ID NO:24 (CDR1), SEQ ID NO:25 (CDR2) and SEQ ID NO:26 (CDR3).

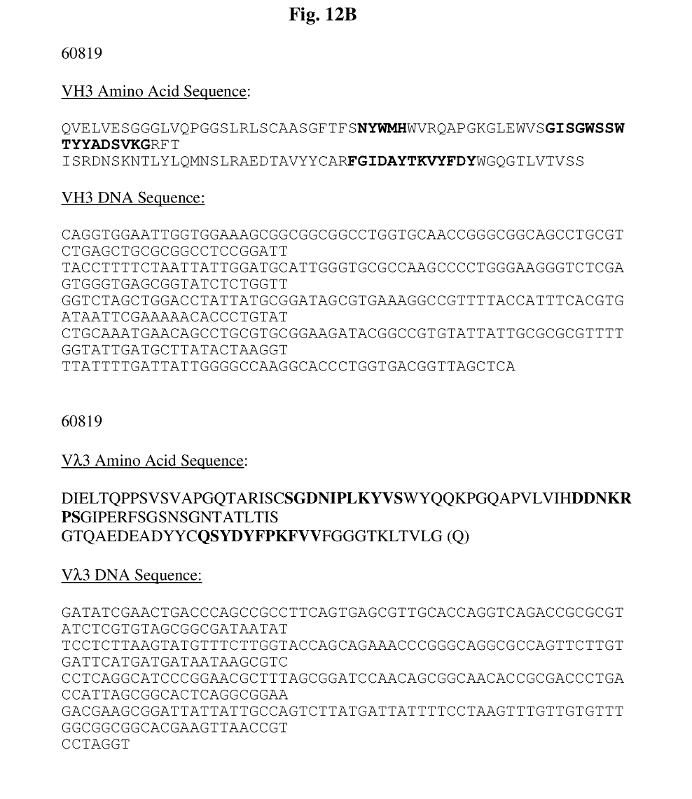

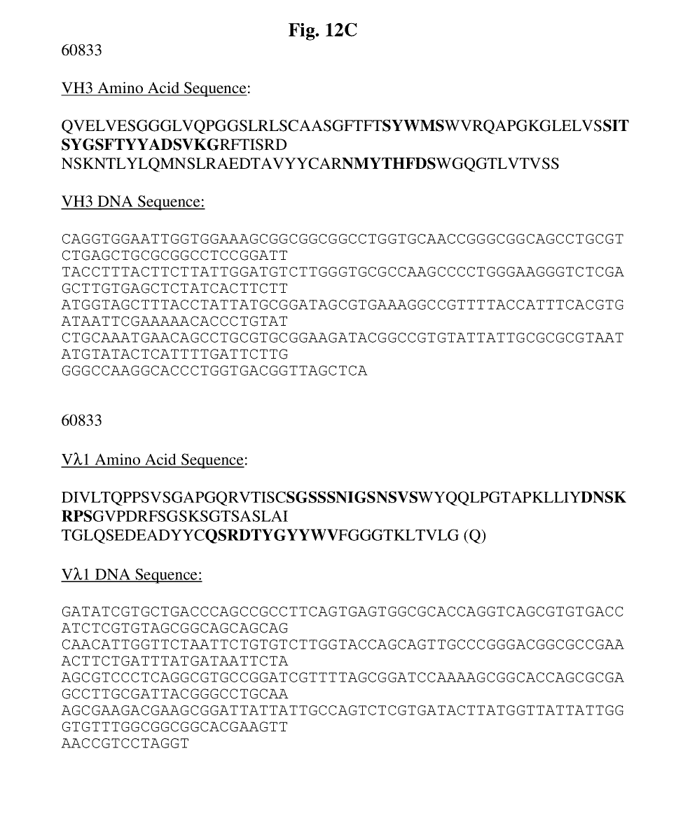

In another particular embodiment, an anti-IGF antibody within this invention refers to an anti-IGF antibody molecule having heavy and light chains or CDRs having amino acid sequences as depicted in FIG. 12A-C.

In another particular embodiment, an anti-IGF antibody within this invention refers to an anti-IGF antibody molecule, wherein said antibody molecule binds to a nonlinear epitope within IGF-1 comprising the amino acid sequences LCGAELVDALQFVCGDR (SEQ ID NO:41) and CCFRSCDLRRLEM (SEQ ID NO:42) of human IGF-1 (SEQ ID NO:43). In a preferred embodiment, said antibody molecule makes contact with at least 8 amino acids within the amino acid sequence LCGAELVDALQFVCGDR (SEQ ID NO:41), and at least 10 amino acids within amino acid sequence CCFRSCDLRRLEM (SEQ ID NO:42) of human IGF-1 (SEQ ID NO:43). In a further preferred embodiment, such anti-IGF antibody molecule makes contact with Leu (5), Cys (6), Glu (9), Leu (10), Asp (12), Ala (13), Phe (16), Val (17), Arg (21), Cys (47), Cys (48), Phe (49), Ser (51), Cys (52), Asp (53), Leu (54), Arg (55), Leu (57), and Glu (58) of human IGF-1 (SEQ ID NO:43), as determined by X-ray crystallography. A respective method is disclosed in Example 9 herein. Preferably, said antibody molecule has heavy chain CDRs comprising the amino acid sequences of SEQ ID NO:21 (CDR1), SEQ ID NO:22 (CDR2) and SEQ ID NO:23 (CDR3) and has light chain CDRs comprising the amino acid sequences of SEQ ID NO:24 (CDR1), SEQ ID NO:25 (CDR2) and SEQ ID NO:26 (CDR3).

Binding of the antibody is defined as the interaction that occurs via the non-covalent bonds that hold the antigen (or a protein or a fragment thereof that is structurally similar) to the antibody combining site, i.e. the region of the immunoglobulin that combines with the determinant of an appropriate antigen (or a structurally similar protein).

Affinity (i.e. the interaction between a single antigen-binding site on an antibody and a single epitope) is expressed by the association constant K_{A}=kₐₛₛ/K_{diss} or the dissociation constant K_{D}=k_{diss}/kₐₛₛ.

In one embodiment according to a), the antibody binds to each IGF protein with an affinity, as determined by surface plasmon resonance analysis, with a K_{D} value ranging from 0.02 nM to 20 nm, e.g. 0.2 nM to 2 nM, for example, with an affinity of 0.05, 0.1, 0.2, 0.3, 0.4, 0.5, 0.6, 0.7, 0.8, 0.9, or 1.0 nM. Based on this property, neutralization of IGF functional signaling is achieved.

In one embodiment according to c), the antibody does not bind to human insulin at concentrations that are at least 100-fold higher than the minimum concentration required for binding to human IGF-1 or IGF-2.

In another embodiment, the property of the anti-IGF antibody molecule defined in c) is characterized by the fact that the affinity of the anti-IGF antibody molecule to IGF-1 and IGF-2, respectively, is at least 100-fold, and even more than 1000-fold, as compared to its affinity to insulin. Even though at very high doses, e.g. more than 100 mg/kg, weak binding may not be completely excluded, the anti-IGF antibody molecule does not bind to insulin at therapeutic doses.

In one embodiment, the antibody molecules of the invention do not affect the mitogenic properties of human insulin that are mediated by its binding to the insulin receptor. (In general, a mitogenic property is defined as the ability of a compound to encourage a cell to commence cell division, triggering mitosis, e.g. in the case of insulin, its ability to promote cell growth).

In another embodiment, in addition to its ability to inhibit IGF signaling mediated via the IGF-1 receptor, an antibody of the invention also has the ability to inhibit IGF-2 signaling mediated via the insulin receptor IR-A.

The antibodies of the invention have a surprisingly high neutralisation potency towards IGF-1 and IGF-2. Furthermore, they have an unexpected higher potency and binding affinity towards IGF-1 than towards IGF-2. They have high solubility and stability, they are free of undesirable glycosylation or hydrolysis motifs in the variable domain, and have a long half-life in the circulation.

In a preferred embodiment, an antibody molecule of the invention, as defined above in i), has a variable heavy chain comprising the amino acid sequence of SEQ ID NO:8 and a variable light chain comprising the amino acid sequence of SEQ ID NO:10 (this sequence may contain, at its C-terminus, an additional Gln. This amino acid position may either be considered the C-terminal end of the variable region, according to the Kabat numbering, or alternatively, and in line with the sequences in the sequence listing, it may represent the first amino acid of the constant light chain, see SEQ ID NO:34).

Preferably, an antibody with the variable heavy chain comprising the amino acid sequence of SEQ ID NO:8 and a variable light chain comprising the amino acid sequence of SEQ ID NO:10 has an IgG1 constant heavy chain region. Preferably, such antibody has an IgX constant light chain region. Preferably, such antibody is the antibody designated 60814, which has a heavy chain constant region which comprises the amino acid sequence of SEQ ID NO:32 and a light chain constant region which comprises the amino acid sequence of SEQ ID NO:34. The complete amino acid sequences of the antibody designated 60814 are depicted in SEQ ID NO:35 (heavy chain) and SEQ ID NO:36 (light chain).

In another preferred embodiment, an antibody molecule of the invention, as defined above in ii), has a variable heavy chain comprising the amino acid sequence of SEQ ID NO:18 and a variable light chain comprising the amino acid sequence of SEQ ID NO:20 (this sequence may contain, at its C-terminus, an additional Gln. This amino acid position may either be considered the C-terminal end of the variable region, according to the Kabat numbering, or alternatively, and in line with the sequences in the sequence listing, it may represent the first amino acid of the constant light chain, see SEQ ID NO:34).

Preferably, an antibody with the variable heavy chain comprising the amino acid sequence of SEQ ID NO:18 and a variable light chain comprising the amino acid sequence of SEQ ID NO:20 has an IgG1 constant heavy chain region. Preferably, such antibody has an Igλ constant light chain region. Preferably, such antibody is the antibody designated 60819, which has a heavy chain constant region which comprises the amino acid sequence of SEQ ID NO:32 and a light chain constant region which comprises the amino acid sequence of SEQ ID NO:34. The complete amino acid sequences of the antibody designated 60819 are depicted in SEQ ID NO:37 (heavy chain) and SEQ ID NO:38 (light chain).

In another preferred embodiment, an antibody of the invention, as defined above in iii), has a variable heavy chain comprising the amino acid sequence of SEQ ID NO:28 and a variable light chain comprising the amino acid sequence of SEQ ID NO:30 (this sequence may contain, at its C-terminus, an additional Gln. This amino acid position may either be considered the C-terminal end of the variable region, according to the Kabat numbering, or alternatively, and in line with the sequences in the sequence listing, it may represent the first amino acid of the constant light chain, see SEQ ID NO:34).

Preferably, an antibody with the variable heavy chain comprising the amino acid sequence of SEQ ID NO:28 and a variable light chain comprising the amino acid sequence of SEQ ID NO:30 has an IgG1 constant heavy chain region. Preferably, such antibody has an Igλ constant light chain region. Preferably, such antibody is the antibody designated 60833, which has a heavy chain constant region which comprises the amino acid sequence of SEQ ID NO:32 and a light chain constant region which comprises the amino acid sequence of SEQ ID NO:34. The complete amino acid sequences of the antibody designated 60833 are depicted in SEQ ID NO:39 (heavy chain) and SEQ ID NO:40 (light chain).

The cross-reactivity of the antibodies of the invention with mouse and rat IGF-1 allows to examine their endocrine effects, e.g. the effect on the growth hormone pathway, in these species. Cross-reactivity with the rat IGFs is particularly advantageous because the rat is an excellent animal model that is preferably used in drug development to study toxicological effects.

The observed pharmacodynamic effect of the antibodies on total IGF-1 levels, likely due to removal of the free IGF-1, which results in feedback regulation through the growth hormone pathway resulting in increased secretion of IGF-1 by the liver, is a useful pharmacodynamic marker. The availability of such marker in animal species, which allows determination of a dose/effect relationship early in drug development, facilitates the preparation of Phase I clinical studies where, in addition to PK analysis, the pharmacodynamic response on total IGF-1 levels in patients are monitored.

The anti-IGF antibody molecule of the invention may also be a variant of an antibody as defined by the amino acid sequences shown in the sequence listing. Thus, the invention also embodies antibodies that are variants of these polypeptides, which have the features a) to c) defined above. Using routinely available technologies, the person skilled in the art will be able to prepare, test and utilize functional variants of the antibodies 60814, 60819 and 60833. Examples are variant antibodies with at least one position in a CDR and/or framework altered, variant antibodies with single amino acid substitutions in the framework region where there is a deviation from the germline sequence, antibodies with conservative amino substitutions, antibodies that are encoded by DNA molecules that hybridize, under stringent conditions, with the DNA molecules presented in the sequence listing encoding antibody variable chains of 60814, 60819 or 60833, functionally equivalent codon-optimized variants of 60814, 60819 and 60833.

A variant may also be obtained by using an antibody of the invention as starting point for optimization and diversifying one or more amino acid residues, preferably amino acid residues in one or more CDRs, and by screening the resulting collection of antibody variants for variants with improved properties. Particularly preferred is diversification of one or more amino acid residues in CDR3 of the variable light chain, CDR3 of the variable heavy chain, CDR1 of the variable light and/or CDR2 of the variable heavy chain. Diversification can be done by methods known in the art, e.g. the so-called TRIM technology referred to in WO 2007/042309.

Given the properties of individual amino acids, rational substitutions can be performed to obtain antibody variants that conserve the overall molecular structure of antibody 60814, 60819 or 60833. Amino acid substitutions, i.e., “conservative substitutions”, may be made, for instance, on the basis of similarity in polarity, charge, solubility, hydrophobicity, hydrophilicity, and/or the amphipathic nature of the respective amino acid. The skilled person is familiar with commonly practiced amino acid substitutions, as described e.g. in WO 2007/042309, and methods for obtaining thus modified antibodies. Given the genetic code and recombinant and synthetic DNA techniques, DNA molecules encoding variant antibodies with one or more conservative amino acid exchanges can be routinely designed and the respective antibodies readily obtained.

Preferred antibody variants have a sequence identity in the variable regions of at least 60%, more preferably, at least 70% or 80%, still more preferably at least 90% and most preferably at least 95%. Preferred antibodies also have a sequence similarity in the variable regions of at least 80%, more preferably 90% and most preferably 95%.

(“Sequence identity” between two polypeptide sequences indicates the percentage of amino acids that are identical between the sequences. “Sequence similarity” indicates the percentage of amino acids that either are identical or that represent conservative amino acid substitutions.)

In a further embodiment, the anti-IGF antibody molecule of the invention is an “affinity matured” antibody.

An “affinity matured” anti-IGF antibody is an anti-IGF antibody derived from a parent anti-IGF antibody, e.g. 60814, 60819 or 60833, that has one or more alterations in one or more CDRs or in which one or more complete CDRs have been replaced, which results in an improvement in the affinity for the antigens, compared to the respective parent antibody. One of the procedures for generating such antibody mutants involves phage display (Hawkins et al., 1992; and Lowman et al., 1991). Briefly, several hypervariable region sites (e.g. 6-7 sites) are mutated to generate all possible amino substitutions at each site. The antibody mutants thus generated are displayed in a monovalent fashion from filamentous phage particles as fusions to the gene III product of M13 packaged within each particle. The phage-displayed mutants are then screened for their biological activity (e.g. binding affinity) as herein disclosed.

Affinity matured antibodies may also be produced by methods as described, for example, by Marks et al., 1992, (affinity maturation by variable heavy chain (VH) and variable light chain (VL) domain shuffling), or Barbas et al., 1994; Shier et al., 1995; Yelton et al., 1995; Jackson et al., 1995; and Hawkins et al., 1992, (random mutagenesis of CDR and/or framework residues). Preferred affinity matured antibodies will have very high affinities, e.g. low picomolar, for the target antigen.

Within the present invention it is also referred to DNA molecules that encode an anti-IGF antibody molecule of the invention. These sequences include, but are not limited to, those DNA molecules encoding antibodies 60814, 60819 and 60833 as shown in the sequence listing: SEQ ID NO:7 and SEQ ID NO:9, respectively, encoding the variable heavy and light chain, respectively, of antibody 60814; SEQ ID NO:17 and SEQ ID NO:19, encoding the variable heavy and light chain, respectively, of antibody 60819; SEQ ID NO:27 and SEQ ID NO:29, encoding the variable heavy and light chain, respectively, of antibody 60833.

The sequences shown in SEQ ID NO:9, SEQ ID NO:19 and 29, encoding the variable light chains, may, at their 3′ end, contain an additional codon for Gln.

Accordingly, within the present invention it is also referred to nucleic acid molecules that hybridize to the DNA molecules set forth in the sequence listing under high stringency binding and washing conditions, as defined in WO 2007/042309, where such nucleic molecules encode an antibody or functional fragment thereof that has properties equivalent or superior to antibody 60814, 60819 or 60833. Preferred molecules (from an mRNA perspective) are those that have at least 75% or 80% (preferably at least 85%, more preferably at least 90% and most preferably at least 95%) homology or sequence identity with one of the DNA molecules described herein.

Yet another class of DNA variants may be defined with reference to the polypeptide they encode. These DNA molecules deviate with respect to their sequence from those depicted in the sequence listing (SEQ ID NOs:7, 17 and 27, or 9, 19, 29, respectively), but encode, due to the degeneracy of the genetic code, antibodies with the identical amino acid sequences of antibodies 60814, 60819 or 60833, respectively. By way of example, in view of expressing antibodies 60814, 60819 or 60833 in eukaryotic cells, the last nine nucleotides, respectively, that encode the last three amino acids of the variable light chains, can be designed to match codon usage in eukaryotic cells. If it is desired to express the antibodies in E. coli, these sequences can be changed to match E. coli codon usage.

Variants of DNA molecules of the invention can be constructed in several different ways, as described in WO 2007/042309.

For producing the recombinant anti-IGF antibody molecules of the invention, the DNA molecules (cDNA and/or genomic DNA) encoding full-length light chain (in the case of antibody 60814, a sequence comprising SEQ ID NO:9 and SEQ ID NO:33) and heavy chain (in the case of antibody 60814, the sequence comprising SEQ ID NO:7 and SEQ ID NO:31), or fragments thereof, are inserted into expression vectors such that the sequences are operatively linked to transcriptional and/or translational control sequences. In the case of antibody 60819, the sequences are those of SEQ ID NO:19 and SEQ ID NO:33, and SEQ ID NO:17 and SEQ ID NO:31, respectively, in the case of antibody 60833, the sequences are those of SEQ ID NO:29 and SEQ ID NO:33, and SEQ ID NO:27 and SEQ ID NO:31, respectively.

For manufacturing the antibodies of the invention, the skilled artisan may choose from a great variety of expression systems well known in the art, e.g. those reviewed by Kipriyanow and Le Gall, 2004.

In another embodiment, within the present invention it is referred to an expression vector containing a DNA molecule comprising the nucleotide sequence encoding the variable heavy chain and/or the variable light chain of an antibody molecule as described above. Preferably, such an expression vector of containing a DNA molecule comprising the nucleotide sequence of SEQ ID NO:7 and/or SEQ ID NO:9, or comprising the sequence of SEQ ID NO:17 and/or SEQ ID NO:19, or comprising the sequence of SEQ ID NO:27 and/or SEQ ID NO:29. Preferably, such an expression vector additionally comprises a DNA molecule encoding the constant heavy chain and/or the constant light chain, respectively, linked to the DNA molecule encoding the variable heavy chain and/or the variable light chain, respectively.

Expression vectors include plasmids, retroviruses, cosmids, EBV derived episomes, and the like. The expression vector and expression control sequences are selected to be compatible with the host cell. The antibody light chain gene and the antibody heavy chain gene can be inserted into separate vectors. In certain embodiments, both DNA sequences are inserted into the same expression vector. Convenient vectors are those that encode a functionally complete human CH (constant heavy) or CL (constant light) immunoglobulin sequence, with appropriate restriction sites engineered so that any VH (variable heavy) or VL (variable light) sequence can be easily inserted and expressed, as described above. In the case of the antibodies with the variable regions of 60814, 60819 and 60833, the constant chain is usually kappa or lambda for the antibody light chain, for the antibody heavy chain, it can be, without limitation, any IgG isotype (IgG1, IgG2, IgG3, IgG4) or other immunoglobulins, including allelic variants.

The recombinant expression vector may also encode a signal peptide that facilitates secretion of the antibody chain from a host cell. The DNA encoding the antibody chain may be cloned into the vector such that the signal peptide is linked in-frame to the amino terminus of the mature antibody chain DNA. The signal peptide may be an immunoglobulin signal peptide or a heterologous peptide from a non-immunoglobulin protein. Alternatively, the DNA sequence encoding the antibody chain may already contain a signal peptide sequence.

In addition to the antibody chain DNA sequences, the recombinant expression vectors carry regulatory sequences including promoters, enhancers, termination and polyadenylation signals and other expression control elements that control the expression of the antibody chains in a host cell. Examples for promoter sequences (exemplified for expression in mammalian cells) are promoters and/or enhancers derived from CMV (such as the CMV Simian Virus 40 (SV40) promoter/enhancer), adenovirus, (e.g., the adenovirus major late promoter (AdMLP)), polyoma and strong mammalian promoters such as native immunoglobulin and actin promoters. Examples for polyadenylation signals are BGH polyA, SV40 late or early polyA; alternatively, 3″UTRs of immunoglobulin genes etc. can be used.

The recombinant expression vectors may also carry sequences that regulate replication of the vector in host cells (e.g. origins of replication) and selectable marker genes. Nucleic acid molecules encoding the heavy chain or an antigen-binding portion thereof and/or the light chain or an antigen-binding portion thereof of an anti-IGF antibody, and vectors comprising these DNA molecules can be introduced into host cells, e.g. bacterial cells or higher eukaryotic cells, e.g. mammalian cells, according to transfection methods well known in the art, including liposome-mediated transfection, polycation-mediated transfection, protoplast fusion, microinjections, calcium phosphate precipitation, electroporation or transfer by viral vectors.

Preferably, the DNA molecules encoding the heavy chain and the light chain are present on two vectors which are co-transfected into the host cell, preferably a mammalian cell.

In a further embodiment, within the present invention it is also referred to a host cell carrying one or more expression vectors as described before, preferably a mammalian cell.

Mammalian cell lines available as hosts for expression are well known in the art and include, inter alia, Chinese hamster ovary (CHO) cells, NSO, SP2/0 cells, HeLa cells, baby hamster kidney (BHK) cells, monkey kidney cells (COS), human carcinoma cells (e.g., Hep G2 and A-549 cells), 3T3 cells or the derivatives/progenies of any such cell line. Other mammalian cells, including but not limited to human, mice, rat, monkey and rodent cells lines, or other eukaryotic cells, including but not limited to yeast, insect and plant cells, or prokaryotic cells such as bacteria may be used. The anti-IGF antibody molecules of the invention are produced by culturing the host cells for a period of time sufficient to allow for expression of the antibody molecule in the host cells.

Thus, in a further embodiment, within the present invention it is also referred to a method for producing an antibody molecule as described, comprising transfecting a mammalian host cell with one or more vectors as described, cultivating the host cell and recovering and purifying the antibody. In another embodiment, within the present invention it is also referred to a method for producing an antibody as described above, comprising obtaining a mammalian host cell comprising one or more vectors as described, and cultivating the host cell. In another embodiment, the method further comprises recovering and purifying the antibody.

Antibody molecules are preferably recovered from the culture medium as a secreted polypeptide or it can be recovered from host cell lysates if for example expressed without a secretory signal. It is necessary to purify the antibody molecules using standard protein purification methods used for recombinant proteins and host cell proteins in a way that substantially homogenous preparations of the antibody are obtained. By way of example, state-of-the art purification methods useful for obtaining the anti-IGF antibody molecule of the invention include, as a first step, removal of cells and/or particulate cell debris from the culture medium or lysate. The antibody is then purified from contaminant soluble proteins, polypeptides and nucleic acids, for example, by fractionation on immunoaffinity or ion-exchange columns, ethanol precipitation, reverse phase HPLC, Sephadex chromatography, chromatography on silica or on a cation exchange resin. As a final step in the process for obtaining an anti-IGF antibody molecule preparation, the purified antibody molecule may be dried, e.g. lyophilized, as described below for therapeutic applications.

In one embodiment, the anti-IGF antibody molecule of the invention may be purified by a sequence of state-of-the art purifications steps comprising affinity chromatography (recombinant Protein A), low pH viral inactivation, depth filtration, cation exchange chromatography, anion exchange chromatography, nanofiltration, and 30 kD ultra/diafiltration (Shukla et al., 2007).

In a further embodiment, within the present invention it is referred to an antibody molecule as described herein for use in medicine.

In a further embodiment, within the present invention it is referred to a pharmaceutical composition containing, as the active ingredient, an anti-IGF antibody molecule, preferably a full antibody, of the invention.

To be used in therapy, the anti-IGF antibody molecule, optionally in combination with one or more other active agents, is included into pharmaceutical compositions appropriate to facilitate administration to animals or humans.

Typical formulations of the anti-IGF antibody molecule can be prepared by mixing the anti-IGF antibody molecule with physiologically acceptable carriers, excipients or stabilizers, in the form of lyophilized or otherwise dried formulations or aqueous solutions or aqueous or non-aqueous suspensions. Carriers, excipients, modifiers or stabilizers are nontoxic at the dosages and concentrations employed. They include buffer systems such as phosphate, citrate, acetate and other anorganic or organic acids and their salts; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; metal complexes (e.g., Zn-protein complexes); and/or ionic or non-ionic surfactants such as TWEEN™ (polysorbates), PLURONICS™ or fatty acid esters, fatty acid ethers or sugar esters. Also organic solvents can be contained in the antibody formulation such as ethanol or isopropanol. The excipients may also have a release-modifying or absorption-modifying function.

The anti-IGF antibody molecules may also be dried (freeze-dried, spray-dried, spray-freeze dried, dried by near or supercritical gases, vacuum dried, air-dried), precipitated or crystallized or entrapped in microcapsules that are prepared, for example, by coacervation techniques or by interfacial polymerization using, for example, hydroxymethylcellulose or gelatin and poly-(methylmethacylate), respectively, in colloidal drug delivery systems (for example, liposomes, albumin microspheres, microemulsions, nano-particles and nanocapsules), in macroemulsions or precipitated or immobilized onto carriers or surfaces, for example by pcmc technology (protein coated microcrystals). Such techniques are disclosed in Remington, 2005.

Naturally, the formulations to be used for in vivo administration must be sterile; sterilization may be accomplished be conventional techniques, e.g. by filtration through sterile filtration membranes.

It may be useful to increase the concentration of the anti-IGF antibody to come to a so-called high concentration liquid formulation (HCLF); various ways to generate such HCLFs have been described.

The anti-IGF antibody molecule may also be contained in a sustained-release preparation. Such preparations include solid, semi-solid or liquid matrices of hydrophobic or hydrophilic polymers, and may be in the form of shaped articles, e.g., films, sticks or microcapsules and may be applied via an application device. Examples of sustained-release matrices include polyesters, hydrogels (for example, poly(2-hydroxyethyl-methacrylate or sucrose acetate butyrate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilization (e.g. as described in WO 89/011297) from acidic solutions, controlling moisture content, using appropriate additives, and developing specific polymer matrix compositions.

Formulations that may also be used for the anti-IGF antibody molecule of the invention are described in U.S. Pat. No. 7,060,268 and U.S. Pat. No. 6,991,790.

The IGF antibody molecule can be incorporated also in other application forms, such as dispersions, suspensions or liposomes, tablets, capsules, powders, sprays, transdermal or intradermal patches or creams with or without permeation enhancing devices, wafers, nasal, buccal or pulmonary formulations, or may be produced by implanted cells or—after gene therapy—by the individual's own cells.

An anti-IGF antibody molecule may also be derivatized with a chemical group such as polyethylene glycol (PEG), a methyl or ethyl group, or a carbohydrate group. These groups may be useful to improve the biological characteristics of the antibody, e.g., to increase serum half-life or to increase tissue binding.

The preferred mode of application of the anti-IGF antibody is parenteral, by infusion or injection (intraveneous, intramuscular, subcutaneous, intraperitoneal, intradermal), but other modes of application such as by inhalation, transdermal, intranasal, buccal, oral, may also be applicable.

In a preferred embodiment, the pharmaceutical composition for administering the anti-IGF-antibody of the invention contains the anti-IGF-antibody, e.g. antibody 60814, 60819 or 60833, in a concentration of 10 mg/ml and further comprises 25 mM Na citrate pH 6, 115 mM NaCl, 0.02% Tween® (polysorbate 20).

In another embodiment, the pharmaceutical composition for administering the anti-IGF-antibody of the invention is an aqueous solution which contains the anti-IGF-antibody, e.g. antibody 60814, 60819 or 60833, in a concentration of 10 mg/ml, and further comprises 25 mM histidine HCl pH 6, 38.8 g/L mannitol, 9.70 g/L sucrose, and 0.02% Tween® (polysorbate 20).

For intravenous infusion, the pharmaceutical composition for administering the anti-IGF-antibody of the invention may be diluted with a physiological solution, e.g. with 0.9% sodium chloride or G5 solution.

The pharmaceutical composition may be freeze-dried and reconstituted with water for injection (WFI) before use.

For the prevention or treatment of disease, the appropriate dosage of antibody will depend on the type of disease to be treated, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to 20 mg/kg (e.g. 0.1-15 mg/kg) of antibody is an initial candidate dosage for administration to the patient, whether, for example, by one or more separate administrations, or by continuous infusion, e.g. infusion over 1 hour. A typical treatment schedule usually involves administration of the antibody once every week to once every three weeks with doses ranging from about 0.1 μg/kg to ca. 20 mg/kg or more, depending on the factors mentioned above. For example, a weekly dose could be 5, 10, or 15 mg/kg. Progress of this therapy is easily monitored by conventional techniques and assays.

### Materials and Methods:

Selection of High Affinity Fully Human Antibodies that Bind IGF-1

Selection of specific Fab fragment clones from the human combinatorial antibody library (HuCAL Gold) (Knappik et al., 2000) that bind human IGF-1 with low nanomolar affinity is performed essentially as described by Rauchenberger et al., 2003, in three panning cycles. In order to identify Fab fragments with improved affinity to human IGF-1, several of these ‘parental’ Fab clones are subjected to ‘in vitro affinity maturation’ essentially as described by Nagy et al., 2002. The L-CDR3 (light chain CDR3) and H-CDR2 (heavy chain CDR2) sequences of each clone are separately diversified by substituting the parental sequence with approximately 10⁸ L-CDR3 and H-CDR2 cassettes from HuCAL (Knappik et al., 2000). Phages are prepared from the resultant ‘maturation libraries’ and each library is subjected to solution pannings on human IGF-1. In order to select the highest affinity human IGF-1 binders, the solution pannings are performed under normal and increased stringency washing conditions according to methods known in the art, with antigen reduction, and with and without blocking by human insulin. The panning outputs after three phage panning rounds are subcloned into a Fab expression vector and the affinity of each Fab for human IGF-1 determined by an electrochemiluminescence-based equilibrium titration technology developed by BioVeris (Witney, Oxfordshire, UK) essentially as described by Haenel et al., 2005. The Fab clones with the best IGF-1 affinities are sequenced, then converted into human IgG1 antibodies as described by Krebs et al., 2001, with subnanomolar affinity to human IGF-1 without any change in specificity compared with the parental antibodies.

### Cloning and Recombinant Expression of IgG1 Antibodies

Variable heavy chain regions (VH) and variable light chain regions (VL) are excised from the Fab expression vectors by restriction enzyme digestion and ligated into compatible restriction enzyme sites of pcDNA3.1 based plasmids containing the human IgG1 heavy chain and human Igλ light chain constant regions respectively. EndoFree plasmid preparations (Qiagen) are prepared and the heavy and light chain plasmids are co-transfected into HEK293 freestyle cells (Invitrogen) at a concentration of 1 mg/L of each plasmid according to the supplier's protocol. After 72 hours the supernatant is harvested and the IgG concentration determined by ELISA. Antibody is purified on a modified protein A column (GE Healthcare), eluted into a citrate buffer and then dialysed to a concentration of 2.5 mg/ml in PBS. Alternatively, a CHO cell line stably integrated with the antibody expression plasmids is generated and used to produce the antibodies.

### Surface Plasmon Resonance Analysis for Determining Affinity Constants

### A) Antibody Capture Method

The sensor chip is coated with approximately 1000 RU of the reference antibody in flow cell 1 and approximately 1000 RU of a rabbit-anti-human Fc-gamma-specific antibody in flow cell 2 using the coupling reagents from an amine coupling kit. A target of 1000 RU is set in the surface preparation wizard of the Biacore 3000 software at a flow rate of 5 μl/min. Running buffer used is HBS-EP. The affinity measurements are made using the following parameters: 20 μl/min flow (HCB running buffer:); 25° C. detection temperature; Fc1, Fc2 flow paths; Fc1, Fc2 detection; anti-IGF-huMAb-capturing: 3 min of a 1 μg/ml solution; 5 min IGF-Ag-association; 5 min IGF-Ag-dissociation; regeneration: 30 sec pulse with 50 mM HCl. The IGF antigens are diluted to 500, 250, 125, 62.5 and 31.3 nM in running buffer (HCB) and the different antigen dilutions are run singly over Fc1 and Fc2 with random order. Blank runs using running buffer only are run in-between. A blank run curve is subtracted from each binding curve before affinity analysis. Data evaluation is performed using the BIAevaluation software (version 4.1, Biacore, Freiburg, Germany). The dissociation and association phases of the kinetics are fitted separately. For the separate fit of the k_{diss} values a time-frame of the initial 200-300 seconds in the dissociation phase is used (range of steady decrease of signal). For the separate fit of the kₐₛₛ values, initial time frames of approx 100 seconds are used (range of steady increase of signal) and for calculation the individual k_{diss} values are used with the 1:1 Langmuir association model. The average values with the standard deviations of the kinetic data together with the corresponding dissociation (K_{D}) and association (K_{A}) constants are calculated.

### b) IGF Coating Method

The determination of binding constants of IGF antibodies to IGF ligands when the sensor chip is coated with IGF ligands is essentially performed as described above except that the sensor chip is coated with 35.1 pg/mm² and 38.5 pg/mm² IGF-1 and IGF-2 respectively. The antibodies are then flowed over the chip at the following concentrations: 50, 25, 12.5, 6.25, 3.12 nM.

### Measurement of Binding to Human, Murine and Rat IGFs and to Human Insulin in Immunosorbent Assays

Fully human IgG1 antibodies that bound with high affinity to IGF-1 are also tested for binding to human IGF-1 in direct immunosorbent assays (ELISA). Assays are performed by coating human IGF-1 (R&D Systems, No. 291-G1) to 96-well Maxisorb plates at a concentration of 0.5 μg/ml overnight at 4° C. (100 μl/well). Coating buffer alone is used as a control for unspecific binding. Wells are then washed once with washing buffer (1×TBS-T) and residual binding sites are blocked with 200 μl blocking buffer for 1 hour at room temperature on an orbital shaker followed by a further wash cycle. Serial three-fold dilutions of each test antibody in blocking buffer are prepared directly on the coated plates. Typical concentrations used are 50, 16.6, 5.6, 1.8, 0.6, 0.2, and 0.07 ng/ml. Blocking buffer alone is used as a positive control. The plates are then incubated for 2 hours at room temperature with agitation. After three wash cycles 100 μl/well of HRPO-conjugated anti-human IgG secondary reagent (Jackson ImmunoResearch Inc.) diluted in blocking buffer is added to all wells. After 2 hours incubation at room temperature with agitation the plates are washed three-times and 100 μl/well of TMB substrate solution (equal amounts of solution A and B) are pipetted into all wells. The plates are incubated for 10-20 min at RT with agitation and then the reaction is stopped by addition of 100 μl/well 1 M phosphoric acid. The absorbance is measured at a wavelength of 450 nm (reference 650 nm).

Binding of the fully human IGF-1 binding IgG1 antibodies to mouse IGF-1 (R&D Systems, No. 791-MG), rat IGF-1 (IBT, No. RU100), human IGF-2 (GroPep, No. FM001), mouse IGF-2 (R&D Systems, No. 792-MG), rat IGF-2 (IBT, No. AAU100), and human insulin (Roche) is also tested as described above for human IGF-1 (except that the concentration of human insulin used for coating is 3 μg/ml).

### In Vitro Cell Proliferation Assays for Determining Neutralization Potency

The MCF-7 breast cancer derived cell line (ATCC, HTB-22) and COLO 205 colon cancer-derived cell line (ATCC # CCL-222) are plated in 96-well plates at a cell density of 1000 cells per well in serum-free RPMI medium. 10 ng/ml of either IGF-1 or IGF-2 is added in the presence or absence of a humanized isotype control antibody that does not bind IGF-1 or IGF-2, or antibodies 60814, 60819, and 60833 at concentrations of 12, 37, 111, 333, 1000 and 3000 ng/ml. Cells are cultured for 5 days then the relative cell number in each well determined using the CellTiter-Glo luminescent cell viability assay (Promega). Luminescence (LU=Luminescence Units) is recorded using a XFluor GENios Pro 4.

### Ewing's Sarcoma-Derived Cell Line Growth Assay

The Ewing's sarcoma-derived cell lines TC-71 (ATCC # ACC516) and SK-ES-1 (ATCC# HTB86) are plated in 96-well plates at a density of 1000 cells per well in DMEM medium containing 1×NEAA, 1× sodium pyruvate, 1× glutamax and 10% fetal calf serum (FCS) and incubated overnight at 37° C. and 5% CO₂ in a humidified atmosphere. The following day a serial dilution of test antibody, humanized isotype control antibody (a humanized IgG1 antibody targeted to CD44-v6) that does not bind IGF-1 or IGF-2, rapamycin, or a combination of rapamycin and test antibody, are added to the cells. The typical concentrations used are 30, 10, 3.3, 1.1, 0.37, and 0.12 g/ml (or 100, 10, 1, 0.1, 0.01, 0.001 nM rapamycin and test antibody for combination studies) and each dilution is performed in triplicate wells. The cells plus antibody are then incubated for 120 hours after which time the relative cell number in each well is determined using the CellTiter-Glo luminescent cell viability assay (Promega). Luminescence (LU=Luminescence Units) is recorded using a XFluor GENios Pro 4 and for data analysis the mean value from triplicate wells is taken and fitted by iterative calculations using a sigmoidal curve analysis program (Graph Pad Prism) with variable Hill slope.

### Western Blot Analysis of Phosphorylated AKT and PTEN Levels

SK-ES-1 cells are plated in 6-well plates in medium containing 10% fetal bovine serum and after overnight incubation they are treated with either 100 nM isotype control antibody (a humanized IgG1 antibody targeted to CD44-v6) that does not bind IGF-1 or IGF-2, 100 nM 60819, 100 nM rapamycin, or a combination of 100 nM 60819 and 100 nM rapamycin. 24 hours later the cells are lysed and the cell lysate frozen after the protein concentration is determined by Bradford assay. Western blotting is performed by applying 30 pg of protein lysates to an SDS PAGE gel (BioRad) and the gel blotted on a Citerian gel blotting sandwich. Western blots are incubated overnight with a rabbit anti-beta actin (control) antibody, a rabbit anti-PTEN antibody (Cell Signaling #9559), or a rabbit anti-phospho-pAKT antibody (Cell Signaling #4060), at 1:5000 (anti-beta actin), 1:1000 (anti-PTEN), or 1:2000 (anti-phosphoAKT) dilutions in 1% milk powder. Following washing in TBS an anti-rabbit IgG HRPO-conjugated secondary antibody (Amersham) is applied for 1 hour and after further washes in TBS antibody reactivity is detected by ECL and captured on Hyperfilm (Amersham).

In Vitro Combination of anti-IGF Antibody with EGFR Inhibitor in NSCLC-Derived Cell Line

The NSCLC-derived cell line A-549 (ATCC# CCL-185) is plated in 96-well plates at a density of 1000 cells per well in RPMI 1640 medium containing 2 mM L-glutamine and 10% fetal bovine serum and incubated overnight at 37° C. and 5% CO₂ in a humidified atmosphere. The following day a serial dilution of test IGF antibody, erlotinib/Tarceva, or a combination of test IGF antibody and erlotinib are added to the cells. The typical concentrations of the test IGF antibody used are 30000, 10000, 3333, 1111, 370,123, 41, 14 ng/mL, and the typical concentration of erlotinib used are 20000, 6667, 2222, 741, 247, 82, 27, 9 nM, and each dilution is performed in triplicate wells. The cells are then incubated for 120 hours after which time the relative cell number in each well is determined using the CellTiter-Glo luminescent cell viability assay (Promega). Luminescence (LU=Luminescence Units) is recorded using a XFluor GENios Pro 4 and for data analysis the mean value from triplicate wells is taken and fitted by iterative calculations using a sigmoidal curve analysis program (Graph Pad Prism) with variable Hill slope.

### Determination of the Effect on Total Murine and Total Rat Serum IGF-1 Levels

Single intravenous (bolus) administrations of 25, 12.5, 6.25, and 3.13 mg/kg of test IGF antibody are given to female athymic NMRI nude mice, 6-8 weeks old (n=5). Single 10 minute intravenous administrations of 30, 100, 200 mg/kg of antibody 60819 are given to male and female Wistar Han rats, 6-8 weeks old (n=4 male, 4 female). Prior to antibody treatment and 24 hours post administration a blood sample is taken, serum collected, and total murine or rat IGF-1 levels determined using the OCTEIA rat/mouse total IGF-1 immunocytometric assay. The assay is performed according to the manufacturer's instructions, absorbance is measured at 450 nm and evaluated using the SoftMax Pro software. A standard curve is used to determine the serum concentration of total IGF-1 in ng/ml. Statistical analysis is performed using the GraphPad Prism software.

### Cell Based IGF-1R Phosphorylation ELISA

Mouse fibroblast cell lines recombinantly expressing human IGF-1R or human IR-A are maintained in DMEM supplemented with 10% heat inactivated FCS, 1 mM sodium pyrovate, 0.075% sodium bicarbonate, MEM NEAA, and 0.3 pg/ml puromycin at 37° C. and 5% CO₂ in a humidified incubator. Cells are detached with trypsin/EDTA, resuspended in growth medium and diluted to 100,000 cells/mL. 100 μL (10,000 cells) are seeded in wells of a sterile 96-well plate and incubated overnight in a humidified incubator at 37° C. and 5% CO₂. The cells are then starved with 100 μL/well assay medium (DMEM supplemented with 0.5% heat inactivated FCS; 1 mM sodium pyrovate, 0.075% sodium bicarbonate, and MEM NEAA) and incubated overnight as before. A range of test antibody concentrations prepared in assay medium is added to the cells, all samples are prepared in triplicate to determine the standard deviation for each assay condition. An IGF-1R antibody, IR-3 (Calbiochem, No. GR11L) is also tested in these experiments. IGF-1 (20 ng/mL final concentration), IGF-2 (100 ng/mL final concentration), or human serum (20% final concentration) is then added and the plates incubated for 30 min in the humidified incubator. Cells are fixed by replacing the growth medium with 4% formaldehyde in PBS for 20 min at RT. After two wash cycles with 300 μL/well wash buffer (PBS with 0.1% Triton X-100) for 5 min (with agitation) the cells are quenched with 100 μL/well 1.2 wt % hydrogen peroxide in wash buffer for 30 minutes at RT. Cells are washed again with 300 μL/well washing buffer and blocked with 100 μL/well blocking buffer (5% BSA in wash buffer) for 60 min at RT with agitation. Blocking buffer is removed and 50 μl/well primary phopho-IGF-I receptor (tyr1135/1136)/insulin receptor (tyr1150/1151) antibody (Cell Signaling, No. 3024) diluted 1:1000 in blocking buffer is added. Plates are incubated overnight at 4° C. with agitation then washed three times as above and 50 μL/well anti-rabbit IgG goat immunoglobulins conjugated with horseradish peroxidase (Dako, No. P0448) diluted 1:500 in blocking buffer is added. After a 60 min incubation at RT with agitation the wells are washed twice with washing buffer as above and once with 300 μL PBS. 100 μL/well TMB substrate solution (Bender MedSystems, No. BMS406.1000) is added to the wells and incubated for 10 min with agitation, following this the reaction is stopped by adding 100 μL/well 1 M phosphoric acid and the absorbance read using a photometer (OD 450 nm, OD 650 nm as reference). Inhibition of IGF-1R or IR-A phosphorylation IC₅₀ values are determined by graphical analysis.

### Fab-IGF-1 Co-Crystallisation and Structure Determination

Monoclonal antibodies are prepared in a buffer of 100 mM Na-phosphate (pH 7.0) prior to papain digestion. Papain (Sigma Aldrich, P #3125) is activated in digestion buffer (phosphate buffer containing 10 mM cysteine hydrochloride, 4 mM EDTA, pH 7.0) following the manufacturer's instructions. IgG antibody is mixed with the activated papain (ratio enzyme:IgG=1:100) and the reaction is incubated at 37° C. on a rotor shaker overnight. Digestion is stopped by adding iodacetamid to a final concentration of 30 mM. To separate the Fab fragment from Fc fragments, Fc cleavage products and intact Mab, the digestion mix is loaded onto a Protein A MabSelect column equilibrated with phosphate buffer. The column is washed with 5 column volumes of PBS, and the Fab fragment is collected in the flow-through and wash fractions. The Fc fragment and intact Mab are eluted from the column with 100 mM citrate buffer (pH 3.0) and subsequent size exclusion chromatography of the Fab fragment is performed using a HiLoad Superdex 75 column. The column is run at 0.5 mL per min with 20 mM triethanolamine, 130 mM NaCl, pH 8.0. The protein concentration of Fab fragments is determined by measuring absorbance at 280 nm. Quality of Fab fragments is analysed by Western Blotting and ELISA.

Fab-IGF-1 complex is generated by adding a 2-fold molar excess of the recombinant IGF-1 (Gropep; Receptor Grade) to the purified Fab which is then incubated overnight on a rotor shaker at 4° C. Concentration of the complex to (15 mg/mL) and removal of unbound IGF-1 is performed using an Amicon-Ultra device. Crystallization of the Fab:IGF-1 complex is carried out using various techniques such as hanging drop, sitting drop, and seeding. In one embodiment, the crystal is precipitated by contacting the solution with a reservoir that reduces the solubility of the proteins due to presence of precipitants, i.e., reagents that induce precipitation. Screening of various conditions lead to a suitable buffer system manipulated by addition of a precipitant and additives. The concentration of the precipitants is preferably between 5-50% w/v. The pH of the buffer is preferably about 3 to about 6. The concentration of the protein in the solution is preferably that of super-saturation to allow precipitation. The temperature during crystallization is preferably between 4 and 25° C.

The three dimensional structure of Fab:IGF-1 complex as defined by atomic coordinates is obtained from the X-ray diffraction pattern of the crystal and the electron density map derived there from. The diffraction of the crystals is better than 2 Å resolution. The crystals preferably have the space group P3221 (number 154) and unit cell dimensions of approximately=70 Å, b=70 Å, c=195 Å; and γ=120°. The method for determining the three dimensional structure is molecular replacement which involves use of the structure of a closely related molecule or receptor ligand complex. Model building and refining is done in several iterative steps to final R-factors (R and R_{free}) of 21 and 23% respectively.

### Determination of Pharmacokinetic Parameters in Rats

Wistar rats are given five intravenous bolus administrations of 18, 52, and 248 mg/kg antibody every 72 hours. At various time points a blood sample is taken and the human antibody concentration in the plasma is determined by sandwich ELISA. This allowed the mean pharmacokinetic parameters of the antibody to be calculated on the first day of dosing and half-life is calculated after the last day of dosing (with t(n)=1008 hours).

Selection of High Affinity Antibodies that Bind IGF-1

In order to identify Fab fragments with improved affinity to human IGF-1, several ‘parental’ Fab clones that are identified to bind IGF-1 with low nanomolar affinity are subjected to ‘in vitro affinity maturation’ where the L-CDR3 and H-CDR2 sequences of each clone are separately diversified by substituting the parental sequence with a library of new L-CDR3 and H-CDR2 sequences. The resultant ‘maturation libraries’ are subjected to solution pannings on human IGF-1 and the clones with the best affinity are selected for convertion into IgG1 antibodies and tested further. The three antibodies with the best human IGF-1 affinities are 60814, 60819, and 60833 which had affinities (K_{D}) of 180, 190, and 130 pM respectively (shown in Table 1) as determined by an electrochemiluminescence-based equilibrium titration method.

TABLE 1
IGF-1 binding summary:

| Antibody | Affinity (pM) |
|---|---|
| 60814 | 180 |
| 60819 | 190 |
| 60833 | 130 |

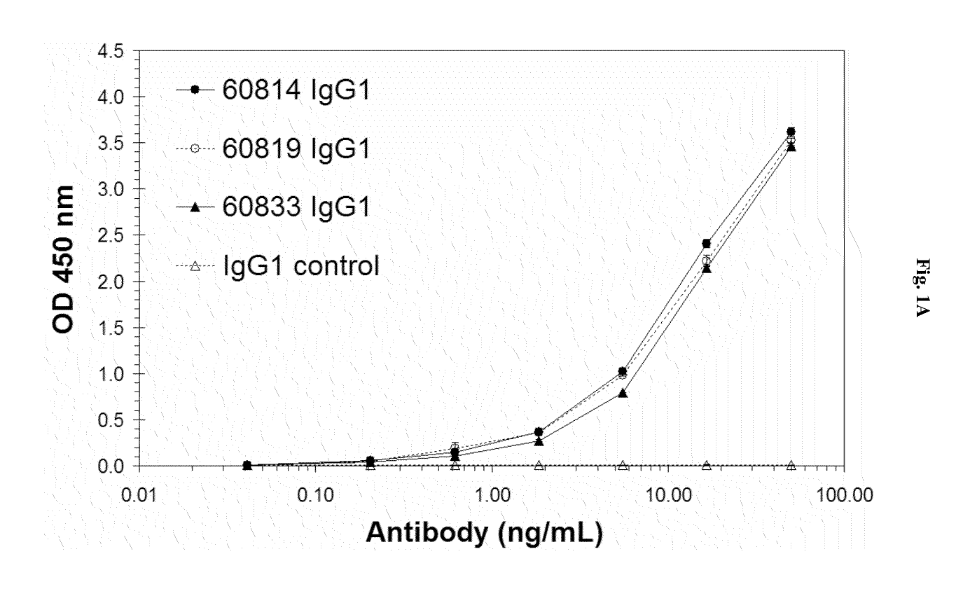

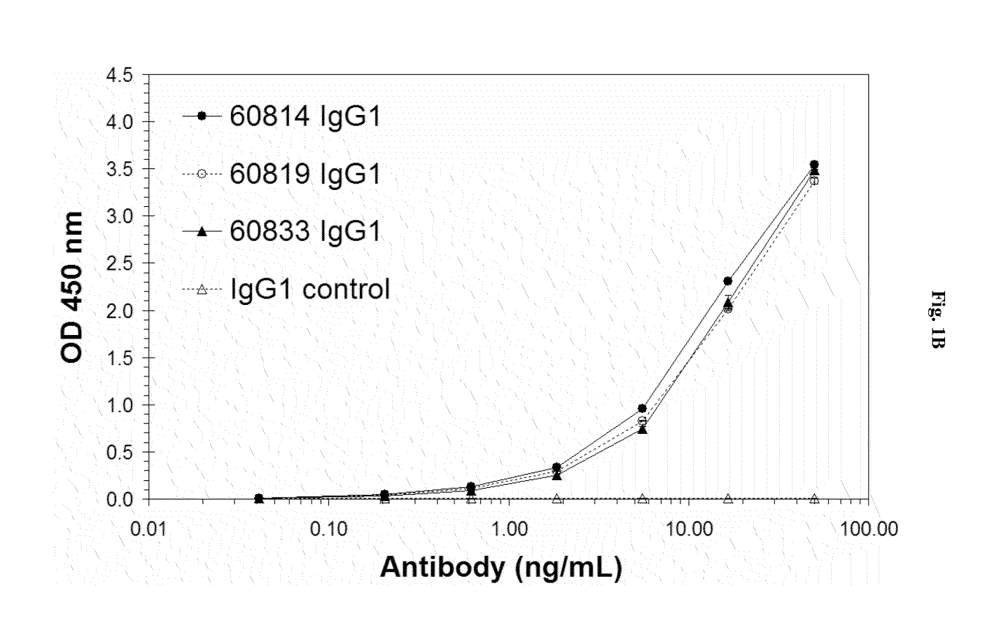

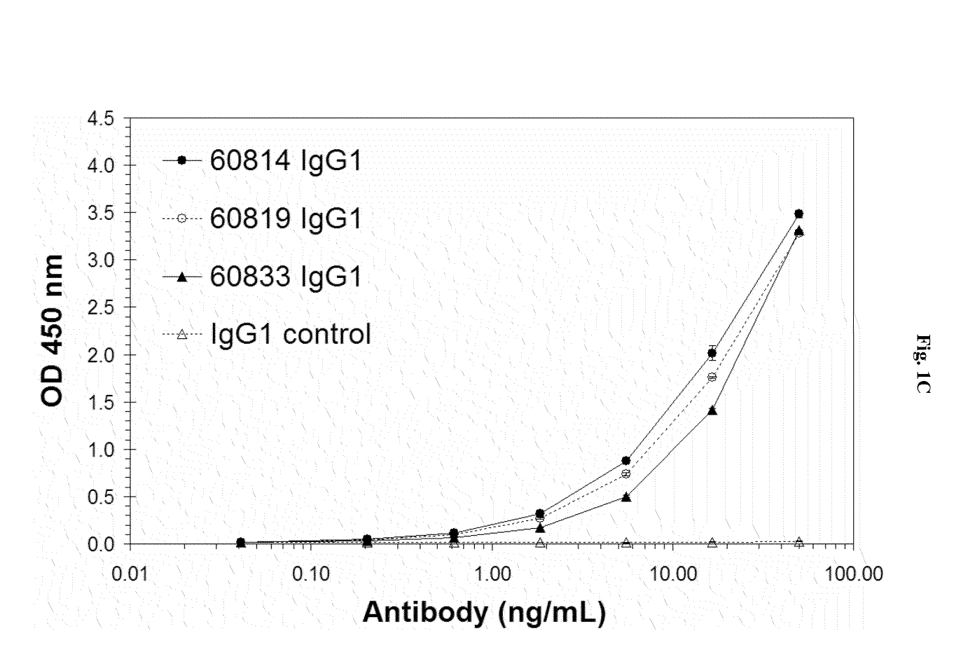

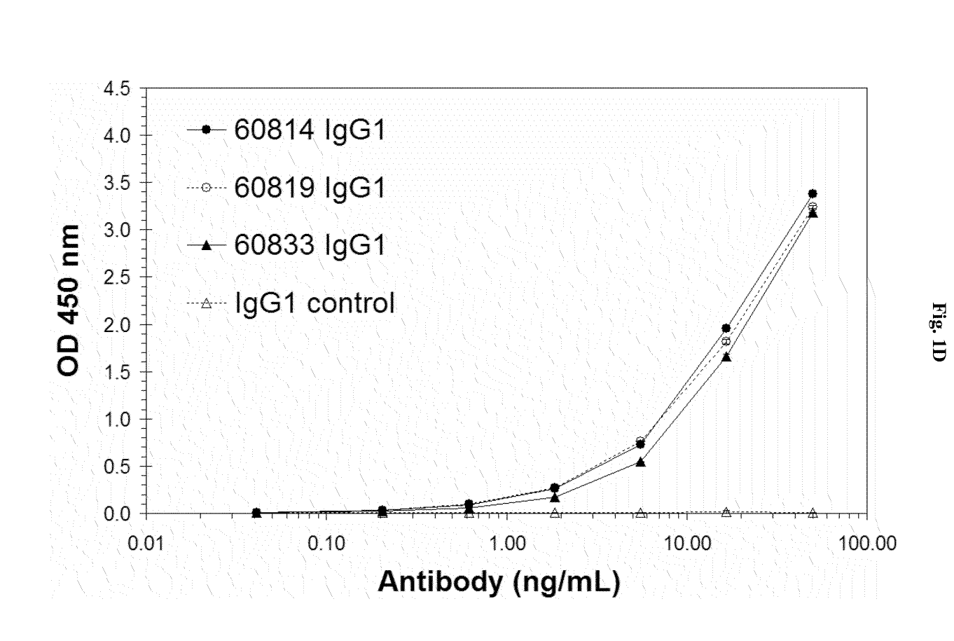

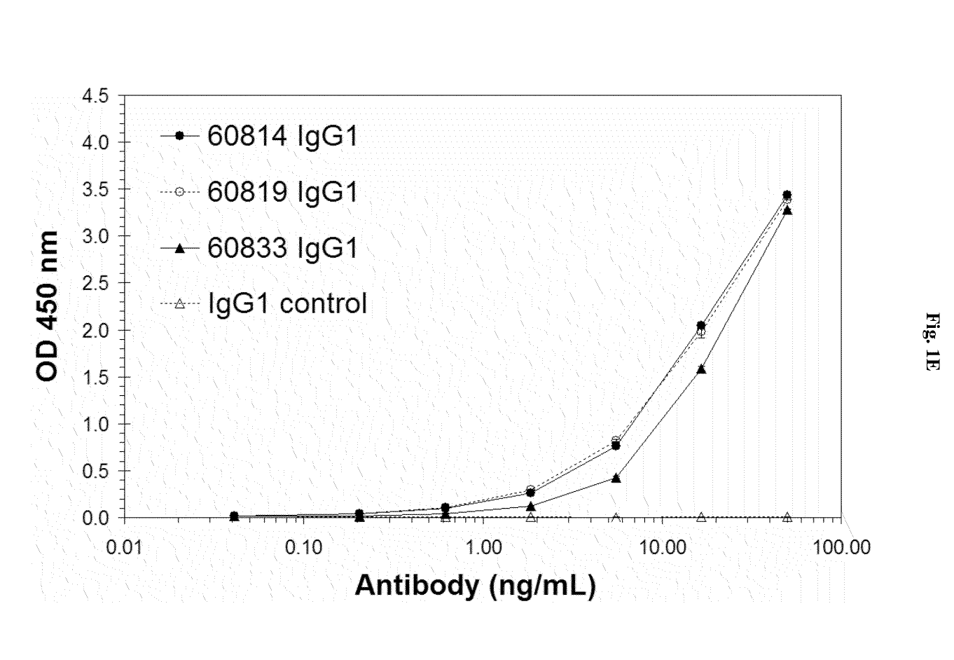

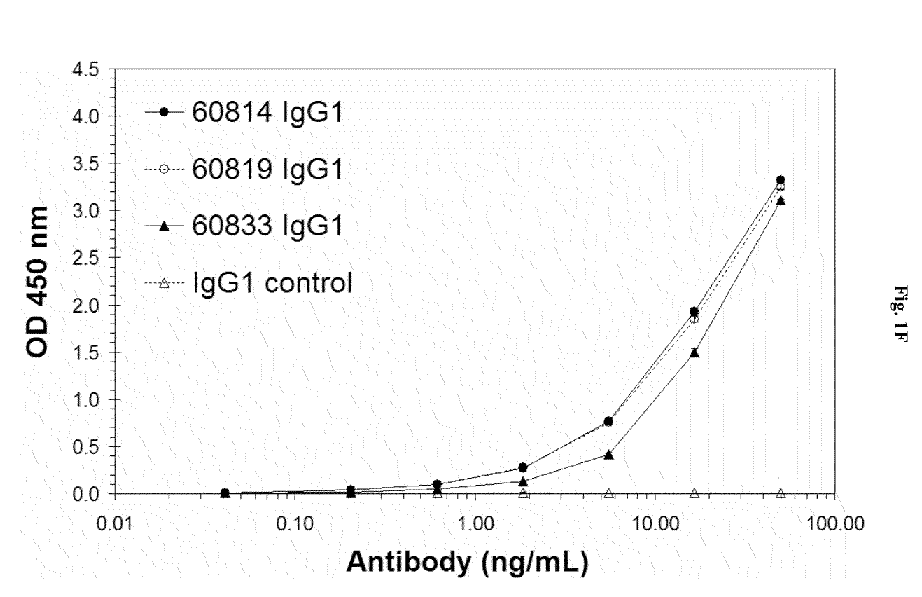

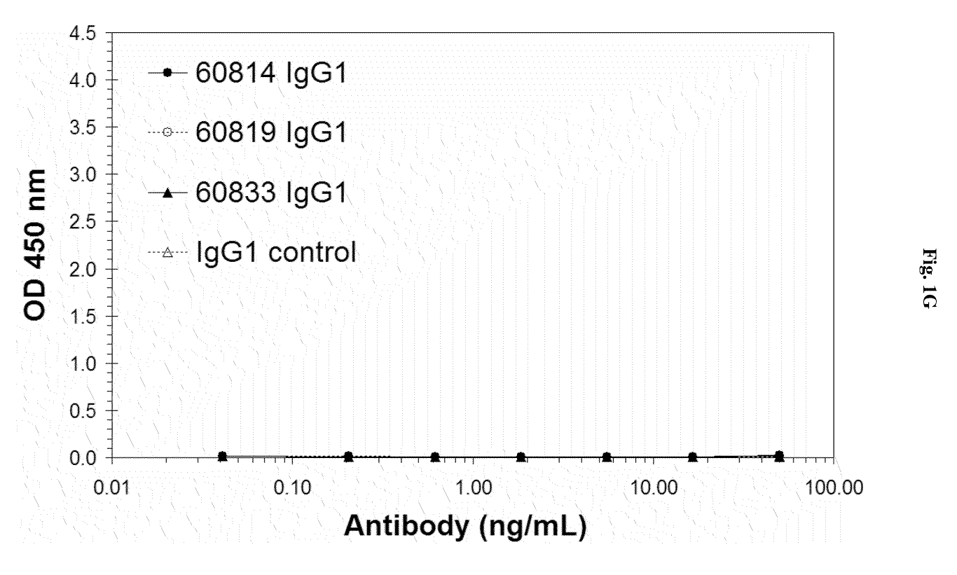

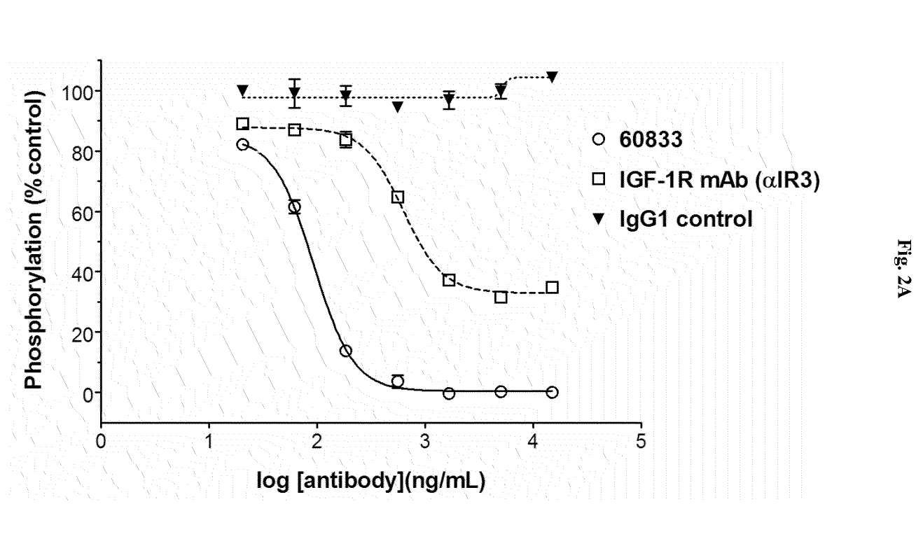

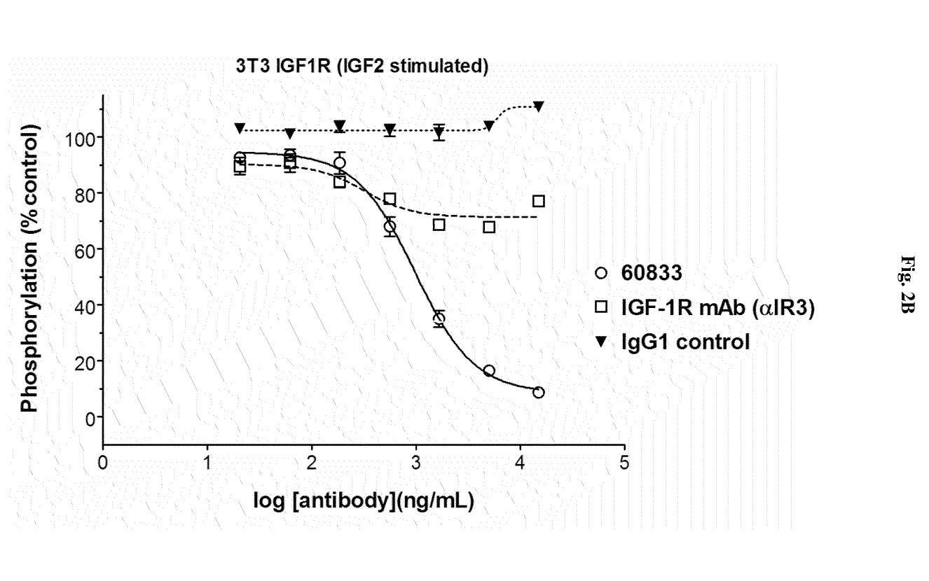

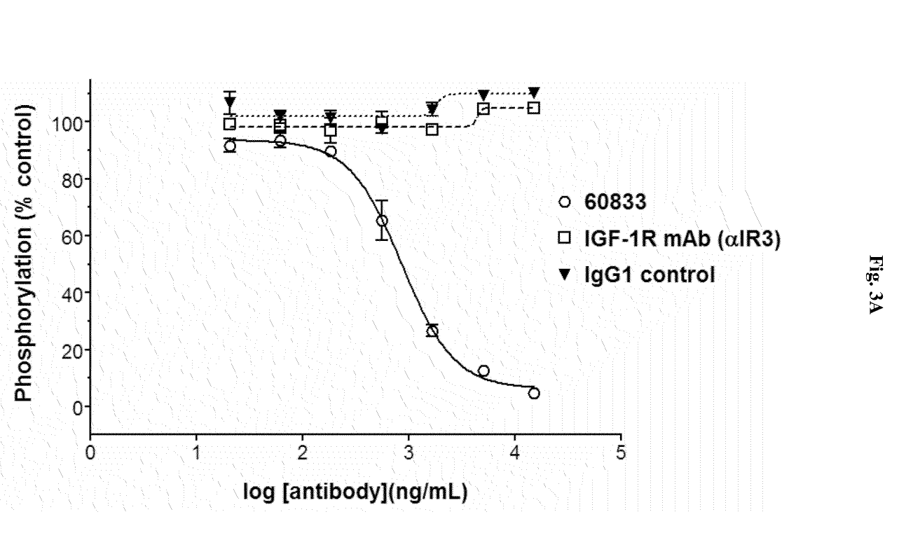

The antibodies are also tested for their binding to human, murine, and rat IGF-1 and IGF-2, and human insulin, in immunosorbent assays. This demonstrated that 60814, 60819, and 60833 show comparable cross-reactive binding with mouse and rat IGF-1, and human, murine and rat IGF-2, but no reactivity to human insulin (at the highest concentration tested, 50 ng/ml) (FIGS. 1A-1G).

Affinity constants for binding of the antibodies to human, mouse, and rat IGF-1 and IGF-2 is also determined by surface plasmon resonance (Biacore) analysis. The method involves capturing the antibodies on the sensor and flowing the IGF antigens over the captured antibodies, thus overcoming any avidity effect that could occur if the IGF antigens are coated onto the sensor and the antibodies added. The affinity constants using this method for antibody 60833 are shown in Table 2 where it can be seen that the measured K_{D} values for human IGF-1 and human IGF-2 are 0.07 nM and 0.9 nM respectively.

TABLE 2
Affinity constants of antibody 60833 for human,
mouse and rat IGF-1 and IGF-2 determined by surface
plasmon resonance (antibody capture method)

| Antigen | Kₒₙ [M⁻¹s⁻¹] | K_{off} [s⁻¹] | K_{D} [nM] |
|---|---|---|---|
| Human IGF-1 | 4.74 × 10⁶ | 3.01 × 10⁻⁴ | 0.07 |
| Mouse IGF-1 | 1.00 × 10⁶ | 3.23 × 10⁻⁴ | 0.33 |
| Rat IGF-1 | 3.81 × 10⁶ | 2.53 × 10⁻⁴ | 0.07 |
| Human IGF-2 | 3.97 × 10⁶ | 3.53 × 10⁻³ | 0.913 |
| Mouse IGF-2 | 8.68 × 10⁵ | 1.1 × 10⁻² | 13.4 |
| Rat IGF-2 | 2.56 × 10⁶ | 6.13 × 10⁻³ | 2.41 |

### Inhibition of IGF Signalling

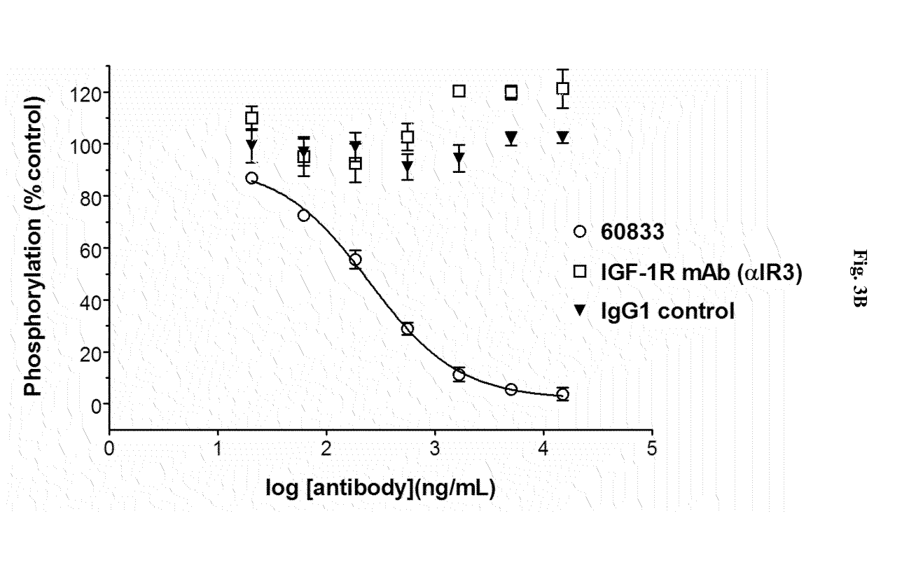

The first signalling event which occurs following binding of IGFs to the IGF-1R is the phosphorylation of the IGF-1R. A cell-based ELISA assay is used to measure the inhibition of IGF induced IGF-1R phosphorylation by the antibody 60833. The potency and effectiveness (of up to 15 μg/mL (100 nM)) of 60833 in neutralising recombinant bioactive IGF-1 and IGF-2 induced IGF-1R phosphorylation is determined. As shown in Table 3 and example FIG. 2 60833 potently and effectively inhibits IGF-1 (FIG. 2A) and IGF-2 (FIG. 2B) induced signalling. In the same assay the IGF-1R targeted mAb IR3 is much less potent and effective with respect to IGF-1 induced signalling, and displays a very weak effect on IGF-2 induced signalling.

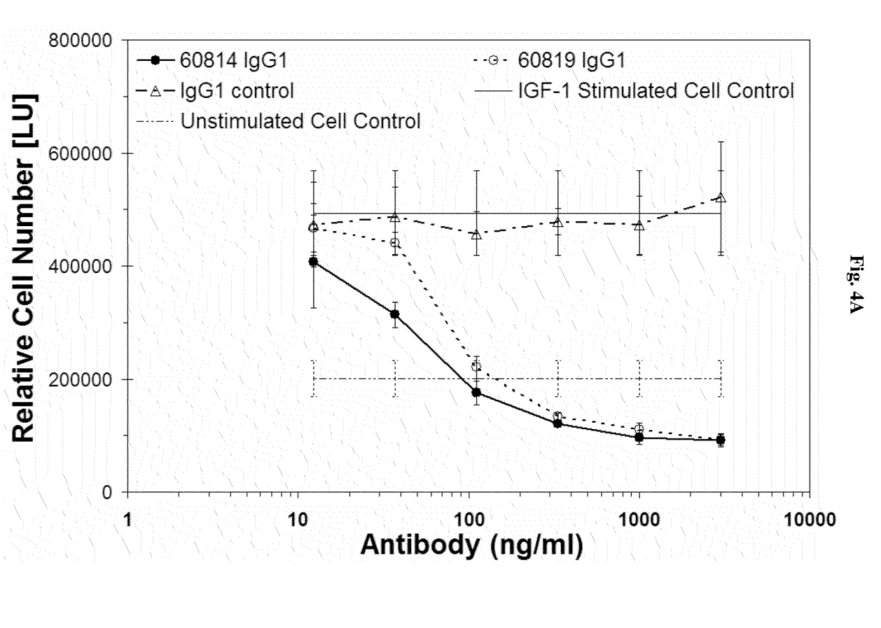

A similar cell based IR-A phosphorylation ELISA is used to demonstrate that 60833 can also inhibit IGF-2 signalling via IR-A. As shown in Table 4 and example FIG. 3A, 60833 potently and effectively inhibits IGF-2 induced IR-A phosphorylation. In contrast, αIR3, which cannot bind IR-A, shows no inhibitory effect.

The level of IGF bioactivity in human serum or plasma samples can also be measured using the IGF-1R phosphorylation cell based ELISA. This is used to determine the potency and effectiveness (up to 15 μg/mL (100 nM)) of 60833 in neutralising human serum IGF bioactivity. As shown in Table 3 and example FIG. 3B 60833 potently and effectively inhibits IGF bioactivity in human serum.

TABLE 3
Effect of 60833 on IGF-1R phosphorylation

| IGF-1R | | | % Remaining |
|---|---|---|---|
| Phosphorylation | | IC₅₀ | Phosphorylation at 15 μg/mL |
| Stimulus | Inhibitor | (μg/mL) | (100 nM) Inhibitor |
| IGF-1 | 60833 | 0.09 | 0 |
| (20 ng/mL) | αIR3 | 1.16 | 35 |
| | Control IgG | >15 | 108 |
| IGF-2 | 60833 | 1.12 | 7 |
| (100 ng/mL) | αIR3 | >15 | 76 |
| | Control IgG | >15 | 108 |
| Human Serum | 60833 | 0.25 | 5 |
| Pooled from | αIR3 | >15 | 120 |
| Healthy Donors | Control IgG | >15 | 110 |
| (20%) | | | |

TABLE 4
Effect of 60833 on IR-A phosphorylation

| IR-A | | | % Remaining |
|---|---|---|---|
| Phosphorylation | | IC₅₀ | Phosphorylation at 15 μg/mL |
| Stimulus | Inhibitor | (μg/mL) | (100 nM) Inhibitor |
| IGF-2 | 60833 | 0.82 | 6 |
| (100 ng/mL) | αIR3 | >15 | 115 |
| | Control IgG | >15 | 109 |

Effects on IGF-1 and IGF-2-induced Cell Proliferation

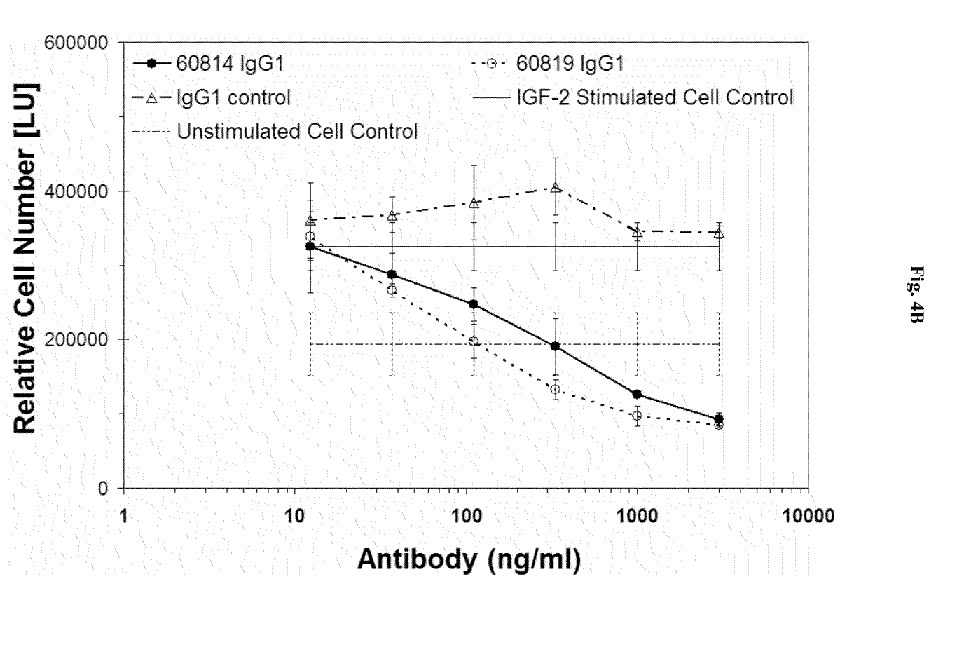

The effects of antibodies 60814, 60819, and 60833 on IGF-1 and IGF-2 induced MCF-7 (breast cancer derived) and COLO 205 (colon cancer derived) cell line proliferation is determined. Examples of the effects of antibodies 60814 and 60819 are shown in FIGS. 4A-D. All three antibodies show a dose dependent inhibition of IGF-1 (FIGS. 4A and 4C) and IGF-2 (FIGS. 4B and 4D) induced MCF-7 (FIGS. 4A and 4B) and COLO 205 (FIGS. 4C and 4D) cell proliferation. The concentration of each antibody required to inhibit 50% of the IGF-1 or IGF-2 induced proliferation of each cell line is shown in Table 5.

TABLE 5
Inhibition of IGF-1 and IGF-2 induced proliferation
of the MCF-7 and COLO 205 cancer cell lines

| | | IC₅₀ (ng/ml) | | |
|---|---|---|---|---|
| Cell Line | Stimulation | 60814 | 60819 | 60833 |
| MCF7 | IGF-1 | 24.1 | 54.0 | 38.6 |
| MCF7 | IGF-2 | 78.2 | 40.8 | 81.2 |
| COLO-205 | IGF-1 | 135.0 | 216.9 | 165.1 |
| COLO 205 | IGF-2 | 576.1 | 100.8 | 632.3 |

### Effects on Proliferation of Ewing's Sarcoma-Derived Cell Lines

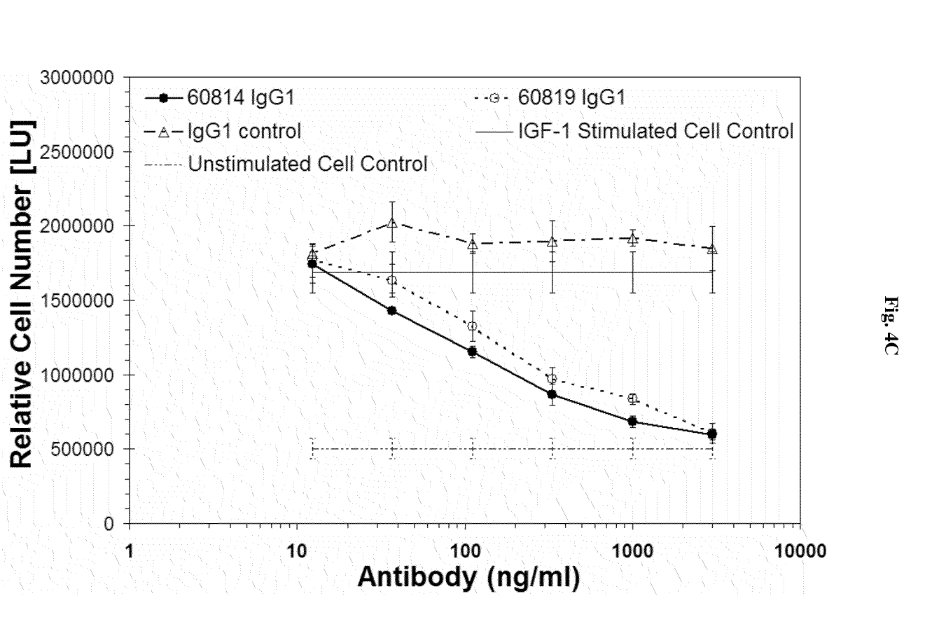

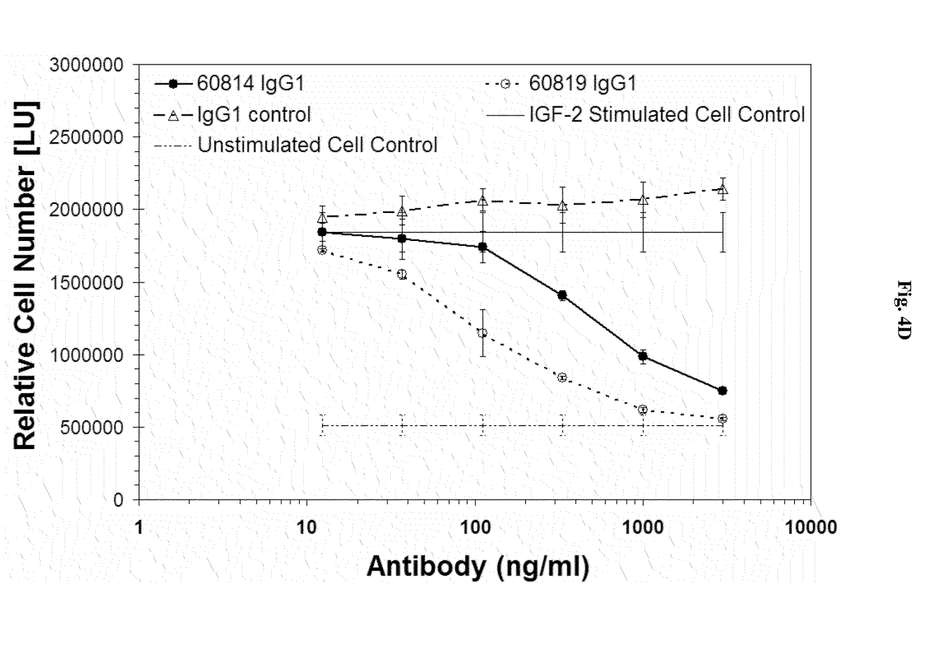

The effect of antibodies 60819 and 60833 on the proliferation of the Ewing's sarcoma-derived cell line TC-71 grown in medium containing 10% FCS is shown in FIG. 5. Relative to a humanized IgG1 isotype control antibody, that does not bind IGF-1 or IGF-2, both 60819 and 60833 show a dose-dependent inhibition of TC-71 cell proliferation.

### Effect on Total Murine and Rat IGF-1 Levels

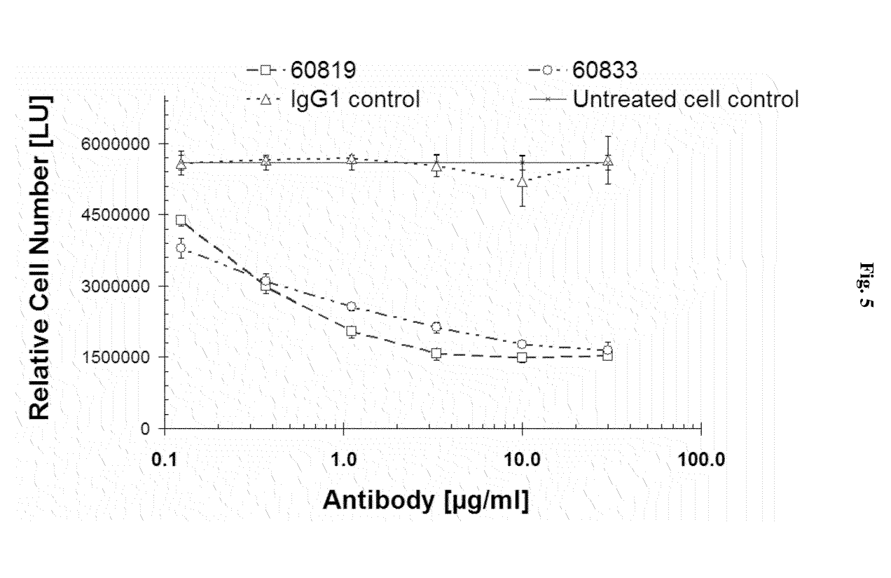

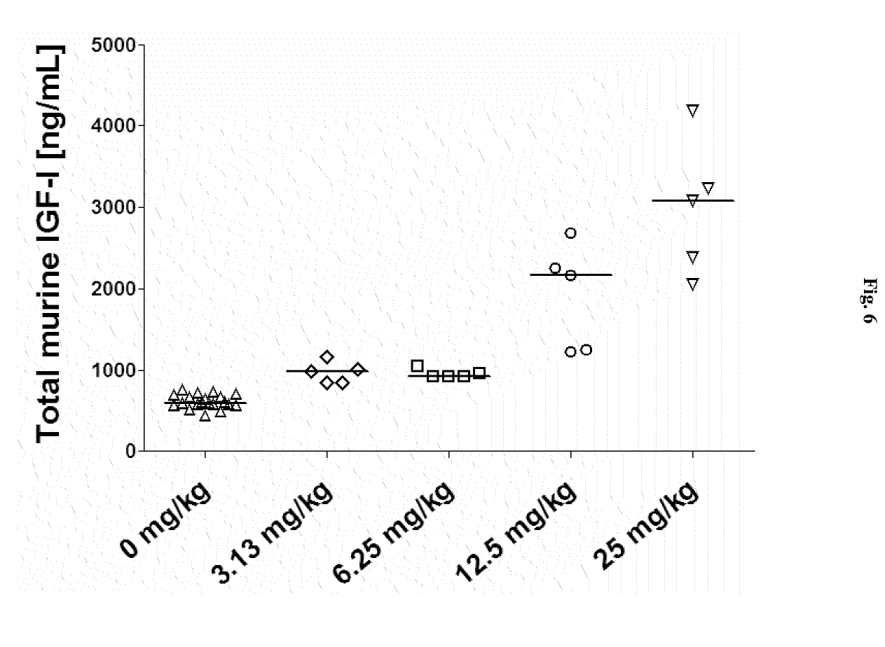

Neutralization of active IGF-1 with an IGF targeted antibody may be expected to result in an endocrine feedback through the GH pathway which results in elevated total serum IGF-1 levels. Antibodies 60814, 60819, and 60833 are cross-reactive with mouse and rat IGF-1 which allows any pharmacodynamic effect on total serum IGF-1 levels to be measured in these species. As shown in FIGS. 6 and 7, administration of antibody 60819 to mice (FIG. 6) and rats (FIG. 7) results in a dose dependent elevation of serum total murine and rat IGF-1 levels 24 hours post administration. This represents a useful pharmacodynamic marker of the activity of these antibodies which can be tested during clinical development in humans.

### Effect of Combination of IGF Ligand Targeting Antibodies and Rapamycin on Ewing's Sarcoma-Derived Cell Line Proliferation and Intracellular Signaling

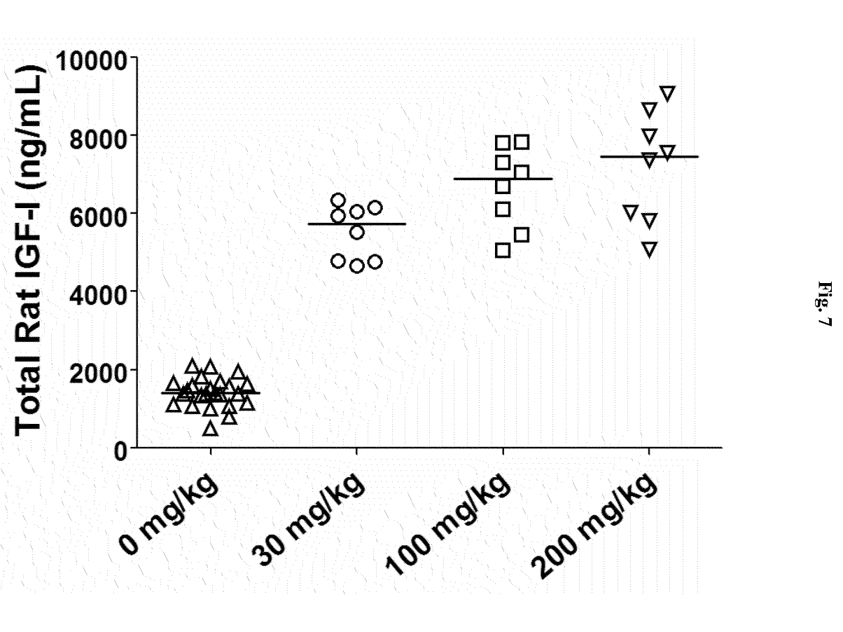

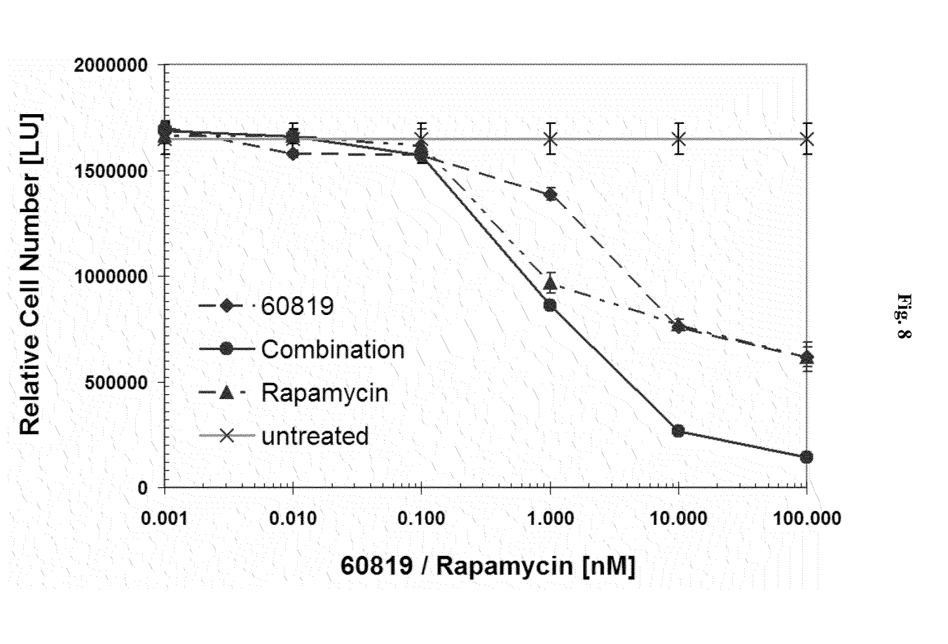

The effect of antibody 60819 and the mTOR inhibitor rapamycin, alone or in combination, on the proliferation of the Ewing's sarcoma-derived cell line SK-ES-1 is shown in FIG. 8. There is a dose dependent inhibition of proliferation with both antibody 60819 and rapamycin alone, with both single agents achieving around 60% proliferation inhibition at 100 nM. Combination of equivalent doses of both antibody 60819 and rapamycin demonstrated an additive effect on the inhibition of cell proliferation with approximately 95% inhibition when 100 nM doses are combined.

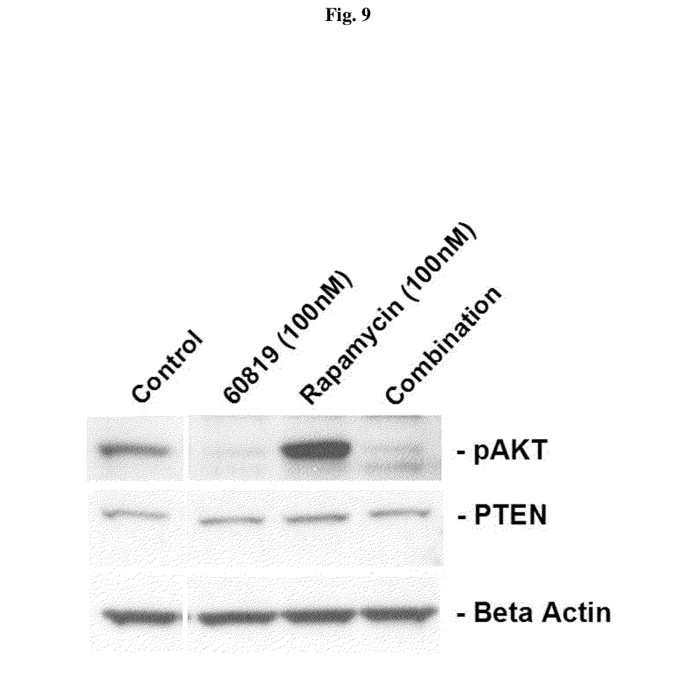

IGF-induced cell proliferation is mediated via a chain of intracellular protein phosphorylation events. One protein whose phosphorylation is increased by IGF stimulation is AKT. FIG. 9 demonstrates the effect of antibody 60819 and rapamycin, alone or in combination, on the phosphorylation of AKT in SK-ES-1 cells 24 hours following treatment using 100 nM doses. Compared with proliferating untreated cells which show phosphorylation of AKT, 100 nM antibody 60819 inhibited AKT phosphorylation. Conversely, 100 nM rapamycin treatment resulted in higher levels of phosphorylated AKT than the control which is thought to be due to a compensatory feedback mechanism following mTOR inhibition. However, when 100 nM rapamycin and 100 nM antibody 60819 are combined the phosphorylation of AKT is inhibited. This suggests that the compensatory feedback which leads to phosphorylated AKT upon rapamycin treatment is due to elevation of the IGF ligands and these are inhibited by antibody 60819. FIG. 9 also demonstrates that both antibody 60819 and rapamycin, alone or in combination, do not affect the total levels of PTEN.

### Effect of Combination of an IGF Ligand Targeting Antibody and an EGFR Inhibitor on NSCLC-Derived Cell Line Proliferation

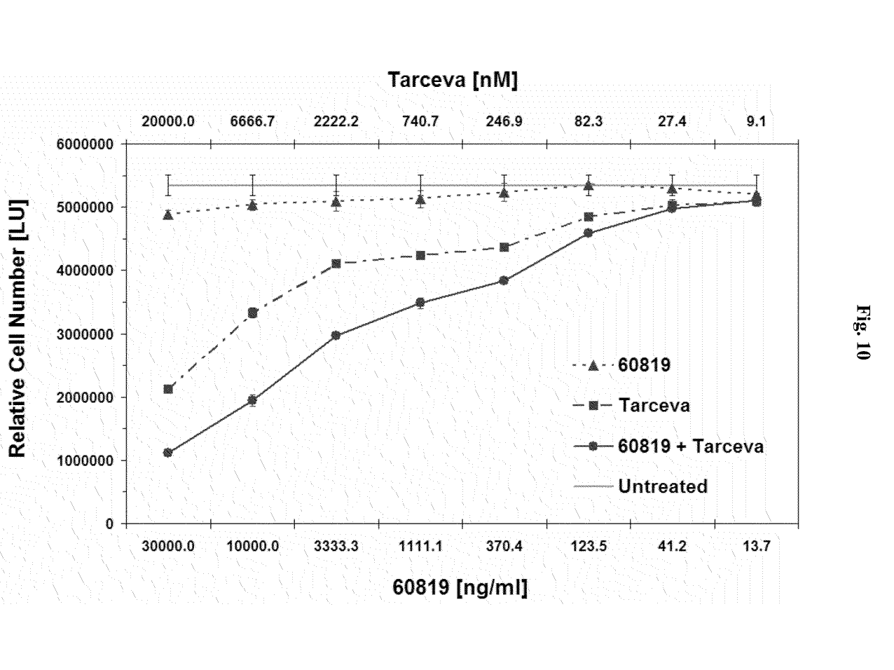

The effect of antibody 60819 and the EGFR inhibitor erlotinib/tarceva, alone or in combination, on the proliferation of the NSCLC-derived cell line A-549 is shown in FIG. 10. In this model, there is only a small effect of antibody 60819 alone on cell proliferation whilst tarceva shows a dose dependent effect with around 60% cell proliferation inhibition at the highest dose tested (20 μM). However, when antibody 60819 and tarceva are combined there is a more potent and effective inhibition of cell proliferation indicative of a synergistic effect.

### Pharmacokinetic Properties in Wistar Rats

The mean pharmacokinetic parameters of antibody 60833 in Wistar rats on the first day of dosing with 18, 52, and 248 mg/kg is shown in Table 6. Terminal half-life was calculated after the last day of dosing (with t(n)=1008 hours), the average terminal half-life for all three dose levels is 221 hr (9.2 days).

TABLE 6
Mean pharmacokinetic parameters of antibody
60833 in Wistar rats on first day of dosing

| | 60833 Dose (mg/kg) | | |
|---|---|---|---|
| | 18 | 52 | 248 |
| C(max) [mg/mL] | 0.531 | 1.70 | 5.56 |
| AUC(0-72 h) [mg · h/mL] | 15.5 | 40.2 | 120 |
| CL [(mL/day)/kg] | 22.9 | 28.7 | 37.3 |
| V(ss) [mL/kg] | 68.1 | 76.3 | 65.4 |
| t½φ [hr] | 210 | 197 | 255 |

φ= after last day of dosing with t(n) = 1008 hr

### Fab-IGF-1 Co-Crystallisation and Structure Determination to Identify Antibody Binding Sites on IGF-1

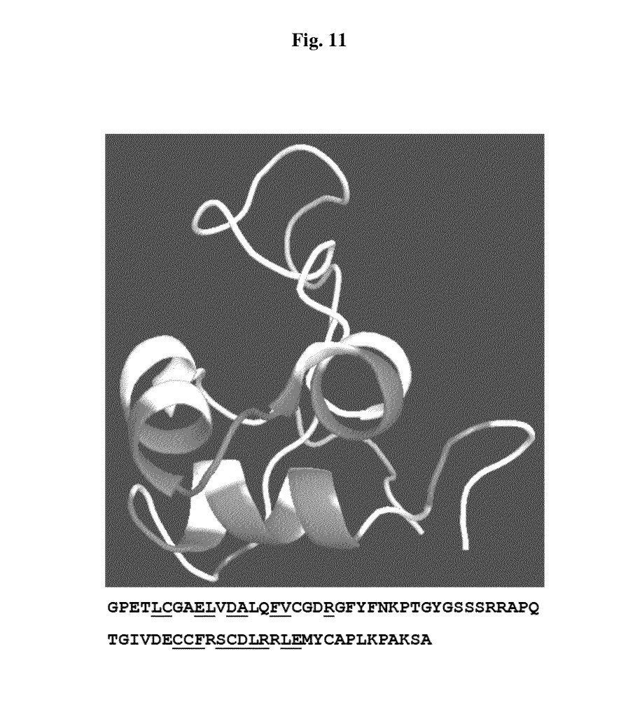

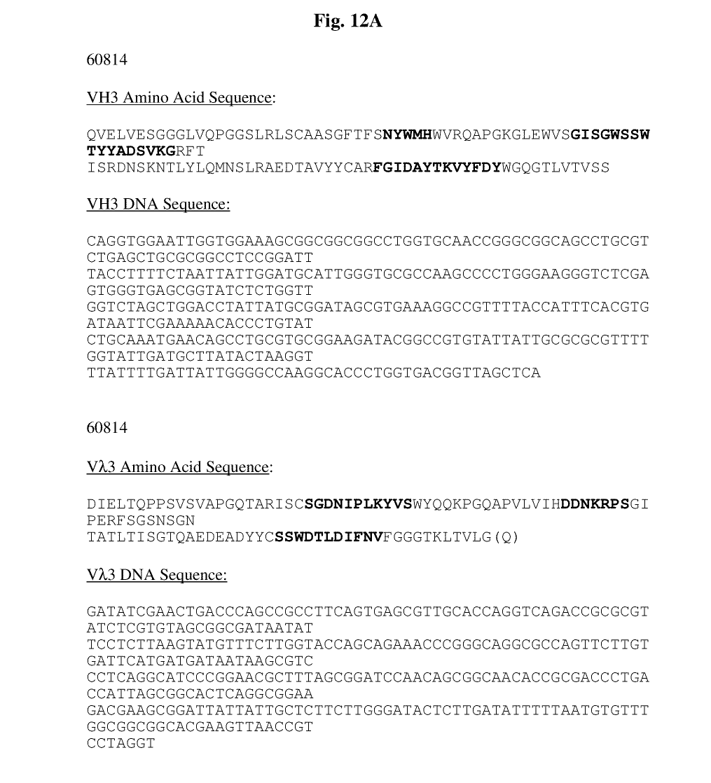

To definitively determine the residues on human IGF-1 that interact with the IGF antibodies the Fab and IGF-1 were co-crystallised and the structure of the interaction determined with better than 2 Å resolution. The residues on IGF-1 that are contacted by antibody (Fab) 60833 are shown in Table 7. In total 19 residues on IGF-1 make contact with 15 CDR residues on 60833. Of these 19 IGF-1 residues 17 are identical in human IGF-2 when the human IGF-1 and IGF-2 amino acid sequences are aligned (listed in Table 7). FIG. 11 shows the 3D structure of IGF-1 with the amino acids that are bound by 60833 highlighted, the linear amino acid sequence of human IGF-1 is also shown with the interacting amino acids underlined.

TABLE 7
Residues in human IGF-1 that make
contacts with residues of 60833 FAB

| IGF-1 residues in | Contact residues on | Homologous |
|---|---|---|
| contact with 60833 | 60833 (CDR) | residue on IGF-2 |
| Leu (L) 5 | Tyr (Y) 54; (HCDR 2) | Leu (L) 8 |
| Cys (C) 6 | Ser (S) 56; (HCDR 2) | Cys (C) 9 |
| Glu (E) 9 | Thr (T) 52; (HCDR 2) | Glu (E) 12 |
| | Ser (S) 53; (HCDR 2) | |
| | Tyr (Y) 54; (HCDR 2) | |
| | Gly (G) 55; (HCDR 2) | |
| | Ser (S) 56; (HCDR 2) | |
| Leu (L) 10 | Phe (F) 57; (HCDR 2) | Leu (L) 13 |
| Asp (D) 12 | Trp (W) 33; (HCDR 1) | Asp (D) 15 |
| Ala (A) 13 | Trp (W) 33; (HCDR 1) | — |
| Phe (F) 16 | Trp (W) 33; (HCDR 1) | Phe (F) 19 |
| | Arg (R) 92; (LCDR 3) | |
| | Tyr (Y) 98; (LCDR 3) | |
| | Trp (W) 99; (LCDR 3) | |
| | Tyr (Y) 101; (HCDR 3) | |
| Val (V)17 | Arg (R) 92; (LCDR 3) | Val (V) 20 |
| | Tyr (Y) 98; (LCDR 3) | |
| Arg (R) 21 | Tyr (Y) 95; (LCDR 3) | Arg (R) 24 |
| Cys (C) 47 | Ser (S) 56; (HCDR 2) | Cys (C) 46 |
| | Phe (F) 57; (HCDR 2) | |
| Cys (C) 48 | Ser (S) 56; (HCDR 2) | Cys (C) 47 |
| Phe (F) 49 | Tyr (Y) 54; (HCDR 2) | Phe (F) 48 |
| | Gly (G) 55; (HCDR 2) | |
| | Ser (S) 56; (HCDR 2) | |
| Ser (S) 51 | Gly (G) 55; (HCDR2) | Ser (S) 50 |
| | Ser (S) 56; (HCDR 2) | |
| | Thr (T) 58; (HCDR 2) | |
| Cys (C) 52 | Ser (S) 56; (HCDR 2) | Cys (C) 51 |
| | Phe (F) 57; (HCDR 2) | |
| | Thr (T) 58; (HCDR 2) | |
| Asp (D) 53 | Phe (F) 57; (HCDR 2) | Asp (D) 52 |
| | Thr (T) 58; (HCDR 2) | |
| Leu (L) 54 | Trp (W) 33; (HCDR 1) | Leu (L) 53 |
| | Phe (F) 57; (HCDR 2) | |
| | Thr (T) 58; (HCDR 2) | |
| | Tyr (Y) 98; (LCDR 3) | |
| Arg (R) 55 | Lys (K) 65; (HCDR 2) | — |
| | Gly (G) 96; (LCDR 3) | |
| | Tyr (Y) 98; (LCDR 3) | |
| Leu (L) 57 | Phe (F) 57; (HCDR 2) | Leu (L) 56 |
| Glu (E) 58 | Tyr (Y) 95; (LCDR 3) | Glu (E) 57 |
| | Gly (G) 96; (LCDR 3) | |
| | Tyr (Y) 98; (LCDR 3) | |
| 19 residues on IGF- | 15 residues on 60833 | |
| 1_involved in | involved in contacts with IGF-1: | |
| contact with 60833 | HCDR 1: 1 residues | |
| | HCDR 2: 8 residues | |
| | HCDR 3: 1 residues | |
| | LCDR 1: — | |
| | LCDR 2: — | |
| | LCDR 3: 5 residues | |

### Combination Therapy

Within this invention it is to be understood that the combinations, compositions, kits, methods or uses according to this invention may envisage the simultaneous, concurrent, sequential, successive, alternate or separate administration of the active ingredients or components. It will be appreciated that the dual Aurora kinase/MEK inhibitor and the anti-IGF antibody can be administered formulated either dependently or independently, such as e.g. the dual Aurora kinase/MEK inhibitor and the anti-IGF antibody may be administered either as part of the same pharmaceutical composition/dosage form or, preferably, in separate pharmaceutical compositions/dosage forms.

In this context, “combination” or “combined” within the meaning of this invention includes, without being limited, fixed and non-fixed (e.g. free) forms (including kits) and uses, such as e.g. the simultaneous, concurrent, sequential, successive, alternate or separate use of the components or ingredients.

The administration of the dual Aurora kinase/MEK inhibitor and the anti-IGF antibody may take place by co-administering the active components or ingredients, such as e.g. by administering them simultaneously or concurrently in one single or in two separate formulations or dosage forms. Alternatively, the administration of the dual Aurora kinase/MEK inhibitor and the Aurora kinase inhibitor may take place by administering the active components or ingredients sequentially or in alternation, such as e.g. in two separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

The elements of the combinations of this invention may be administered (whether dependently or independently) by methods customary to the skilled person, e.g. by oral, enterical, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

Accordingly, in a certain aspect of the invention, the invention provides a method of treating and/or preventing an oncological or hyperproliferative disease, in particular cancer (such as e.g. the cancer disorders described herein), comprising administering to a patient in need thereof a therapeutically effective amount of a dual Aurora kinase/MEK inhibitor and an anti-IGF antibody (each as described herein), simultaneously, concurrently, sequentially, successively, alternately or separately.

In another certain aspect, the invention provides a dual Aurora kinase/MEK inhibitor as described herein for use in a method of treating and/or preventing an oncological or hyperproliferative disease, in particular cancer, said method comprising administering the dual Aurora kinase/MEK inhibitor simultaneously, concurrently, sequentially, successively, alternately or separately with an anti-IGF antibody as described herein.

In another certain aspect, the invention provides an anti-IGF antibody as described herein for use in a method of treating and/or preventing an oncological or hyperproliferative disease, in particular cancer, said method comprising administering the anti-IGF antibody simultaneously, concurrently, sequentially, successively, alternately or separately with a dual Aurora kinase/MEK inhibitor as described herein.

In another certain aspect, the invention provides the use of a dual Aurora kinase/MEK inhibitor and/or an anti-IGF antibody, each as described herein, for preparing a pharmaceutical composition for treating and/or preventing an oncological or hyperproliferative disease, in particular cancer (such as e.g. a cancer disease as described herein), in combination.

In another certain aspect, the invention provides a combination, composition or kit comprising, consisting of, or consisting essentially of a dual Aurora kinase/MEK inhibitor and an anti-IGF antibody, each as described herein, and optionally one or more pharmaceutically acceptable carriers, excipients and/or vehicles, e.g. for simultaneous, concurrent, sequential, successive, alternate or separate use of the active components in therapy.

In a preferred embodiment, the dual dual Aurora kinase/MEK inhibitor is to be administered orally.

In another preferred embodiment, the anti-IGF antibody is to be administered parenterally, by infusion or injection.

The “therapeutically effective amount” of the active compound(s) to be administered is the minimum amount necessary to prevent, ameliorate, or treat a disease or disorder.

The combinations of this invention may be administered at therapeutically effective single or divided daily doses. The active components of the combination may be administered in such doses which are therapeutically effective in monotherapy, or in such doses which are lower than the doses used in monotherapy, but when combined result in a desired therapeutically effective amount.

In certain particular embodiments of this invention, the combinations, compositions, kits, methods and uses according to this invention relate to such combinations, compositions, kits, methods and uses in which the dual Aurora kinase/MEK inhibitor is selected from the Group X consisting of the compounds 1 to 25 indicated herein above and the anti-IGF antibody is an anti-IGF antibody molecule having heavy chain CDRs comprising the amino acid sequences of SEQ ID NO:1 (CDR1), SEQ ID NO:2 (CDR2) and SEQ ID NO:3 (CDR3) and light chain CDRs comprising the amino acid sequences of SEQ ID NO:4 (CDR1), SEQ ID NO:5 (CDR2) and SEQ ID NO:6 (CDR3).

In certain more particular embodiments of this invention, the combinations, compositions, kits, methods and uses according to this invention relate to such combinations, compositions, kits, methods and uses in which the dual Aurora kinase/MEK inhibitor is selected from the Group X consisting of the compounds 1 to 25 indicated herein above and the anti-IGF antibody is an anti-IGF antibody molecule having a variable heavy chain comprising the amino acid sequence of SEQ ID NO:8 and a variable light chain comprising the amino acid sequence of SEQ ID NO:10.

In certain preferred embodiments of this invention, the combinations, compositions, kits, methods and uses according to this invention relate to such combinations, compositions, kits, methods and uses in which the dual Aurora kinase/MEK inhibitor is selected from the Group X consisting of the compounds 1 to 25 indicated herein above and the anti-IGF antibody is an anti-IGF antibody molecule having a heavy chain comprising the amino acid sequence of SEQ ID NO:35 and a light chain comprising the amino acid sequence of SEQ ID NO:36.

In certain particular embodiments of this invention, the combinations, compositions, kits, methods and uses according to this invention relate to such combinations, compositions, kits, methods and uses in which the dual Aurora kinase/MEK inhibitor is selected from the Group X consisting of the compounds 1 to 25 indicated herein above and the anti-IGF antibody is an anti-IGF antibody molecule having heavy chain CDRs comprising the amino acid sequences of SEQ ID NO:11 (CDR1), SEQ ID NO:12 (CDR2) and SEQ ID NO:13 (CDR3) and light chain CDRs comprising the amino acid sequences of SEQ ID NO:14 (CDR1), SEQ ID NO:15 (CDR2) and SEQ ID NO:16 (CDR3).

In certain more particular embodiments of this invention, the combinations, compositions, kits, methods and uses according to this invention relate to such combinations, compositions, kits, methods and uses in which the dual Aurora kinase/MEK inhibitor is selected from the Group X consisting of the compounds 1 to 25 indicated herein above and the anti-IGF antibody is an anti-IGF antibody molecule having a variable heavy chain comprising the amino acid sequence of SEQ ID NO:18 and a variable light chain comprising the amino acid sequence of SEQ ID NO:20.

In certain preferred embodiments of this invention, the combinations, compositions, kits, methods and uses according to this invention relate to such combinations, compositions, kits, methods and uses in which the dual Aurora kinase/MEK inhibitor is selected from the Group X consisting of the compounds 1 to 25 indicated herein above and the anti-IGF antibody is an anti-IGF antibody molecule having a heavy chain comprising the amino acid sequence of SEQ ID NO:37 and a light chain comprising the amino acid sequence of SEQ ID NO:38.

In certain particular embodiments of this invention, the combinations, compositions, kits, methods and uses according to this invention relate to such combinations, compositions, kits, methods and uses in which the dual Aurora kinase/MEK inhibitor is selected from the Group X consisting of the compounds 1 to 25 indicated herein above and the anti-IGF antibody is an anti-IGF antibody molecule having heavy chain CDRs comprising the amino acid sequences of SEQ ID NO:21 (CDR1), SEQ ID NO:22 (CDR2) and SEQ ID NO:23 (CDR3) and has light chain CDRs comprising the amino acid sequences of SEQ ID NO:24 (CDR1), SEQ ID NO:25 (CDR2) and SEQ ID NO:26 (CDR3).

In certain more particular embodiments of this invention, the combinations, compositions, kits, methods and uses according to this invention relate to such combinations, compositions, kits, methods and uses in which the dual Aurora kinase/MEK inhibitor is selected from the Group X consisting of the compounds 1 to 25 indicated herein above and the anti-IGF antibody is an anti-IGF antibody molecule having a variable heavy chain comprising the amino acid sequence of SEQ ID NO:28 and a variable light chain comprising the amino acid sequence of SEQ ID NO:30.

In certain preferred embodiments of this invention, the combinations, compositions, kits, methods and uses according to this invention relate to such combinations, compositions, kits, methods and uses in which the dual Aurora kinase/MEK inhibitor is selected from the Group X consisting of the compounds 1 to 25 indicated herein above and the anti-IGF antibody is an anti-IGF antibody molecule having a heavy chain comprising the amino acid sequence of SEQ ID NO:39 and a light chain comprising the amino acid sequence of SEQ ID NO:40.

In certain embodiments (embodiments A1, B1 and C1) of this invention, the combinations, compositions, kits, methods and uses according to this invention refer to such individual pairs of the dual Aurora kinase/MEK inhibitor and the anti-IGF antibody according to the embodimental entries A1.1 to A1.25, B1.1 to B1.25, and C1.1 to C1.25 of the following Table i:

TABLE i

| Em- | | |
|---|---|---|
| bodi- | dual Aurora kinase/MEK | |
| ment | inhibitor | anti-IGF antibody |
| A1.1 | Compound No. 1 of Group X | Antibody designated as 60814 |
| A1.2 | Compound No. 2 of Group X | Antibody designated as 60814 |
| A1.3 | Compound No. 3 of Group X | Antibody designated as 60814 |
| A1.4 | Compound No. 4 of Group X | Antibody designated as 60814 |
| A1.5 | Compound No. 5 of Group X | Antibody designated as 60814 |
| A1.6 | Compound No. 6 of Group X | Antibody designated as 60814 |
| A1.7 | Compound No. 7 of Group X | Antibody designated as 60814 |
| A1.8 | Compound No. 8 of Group X | Antibody designated as 60814 |
| A1.9 | Compound No. 9 of Group X | Antibody designated as 60814 |
| A1.10 | Compound No. 10 of Group X | Antibody designated as 60814 |
| A1.11 | Compound No. 11 of Group X | Antibody designated as 60814 |
| A1.12 | Compound No. 12 of Group X | Antibody designated as 60814 |
| A1.13 | Compound No. 13 of Group X | Antibody designated as 60814 |
| A1.14 | Compound No. 14 of Group X | Antibody designated as 60814 |
| A1.15 | Compound No. 15 of Group X | Antibody designated as 60814 |
| A1.16 | Compound No. 16 of Group X | Antibody designated as 60814 |
| A1.17 | Compound No. 17 of Group X | Antibody designated as 60814 |
| A1.18 | Compound No. 18 of Group X | Antibody designated as 60814 |
| A1.19 | Compound No. 19 of Group X | Antibody designated as 60814 |
| A1.20 | Compound No. 20 of Group X | Antibody designated as 60814 |
| A1.21 | Compound No. 21 of Group X | Antibody designated as 60814 |
| A1.22 | Compound No. 22 of Group X | Antibody designated as 60814 |
| A1.23 | Compound No. 23 of Group X | Antibody designated as 60814 |
| A1.24 | Compound No. 24 of Group X | Antibody designated as 60814 |
| A1.25 | Compound No. 25 of Group X | Antibody designated as 60814 |
| B1.1 | Compound No. 1 of Group X | Antibody designated as 60819 |
| B1.2 | Compound No. 2 of Group X | Antibody designated as 60819 |
| B1.3 | Compound No. 3 of Group X | Antibody designated as 60819 |
| B1.4 | Compound No. 4 of Group X | Antibody designated as 60819 |
| B1.5 | Compound No. 5 of Group X | Antibody designated as 60819 |
| B1.6 | Compound No. 6 of Group X | Antibody designated as 60819 |
| B1.7 | Compound No. 7 of Group X | Antibody designated as 60819 |
| B1.8 | Compound No. 8 of Group X | Antibody designated as 60819 |
| B1.9 | Compound No. 9 of Group X | Antibody designated as 60819 |
| B1.10 | Compound No. 10 of Group X | Antibody designated as 60819 |
| B1.11 | Compound No. 11 of Group X | Antibody designated as 60819 |
| B1.12 | Compound No. 12 of Group X | Antibody designated as 60819 |
| B1.13 | Compound No. 13 of Group X | Antibody designated as 60819 |
| B1.14 | Compound No. 14 of Group X | Antibody designated as 60819 |
| B1.15 | Compound No. 15 of Group X | Antibody designated as 60819 |
| B1.16 | Compound No. 16 of Group X | Antibody designated as 60819 |
| B1.17 | Compound No. 17 of Group X | Antibody designated as 60819 |
| B1.18 | Compound No. 18 of Group X | Antibody designated as 60819 |
| B1.19 | Compound No. 19 of Group X | Antibody designated as 60819 |
| B1.20 | Compound No. 20 of Group X | Antibody designated as 60819 |
| B1.21 | Compound No. 21 of Group X | Antibody designated as 60819 |
| B1.22 | Compound No. 22 of Group X | Antibody designated as 60819 |
| B1.23 | Compound No. 23 of Group X | Antibody designated as 60819 |
| B1.24 | Compound No. 24 of Group X | Antibody designated as 60819 |
| B1.25 | Compound No. 25 of Group X | Antibody designated as 60819 |
| C1.1 | Compound No. 1 of Group X | Antibody designated as 60833 |
| C1.2 | Compound No. 2 of Group X | Antibody designated as 60833 |
| C1.3 | Compound No. 3 of Group X | Antibody designated as 60833 |
| C1.4 | Compound No. 4 of Group X | Antibody designated as 60833 |
| C1.5 | Compound No. 5 of Group X | Antibody designated as 60833 |
| C1.6 | Compound No. 6 of Group X | Antibody designated as 60833 |
| C1.7 | Compound No. 7 of Group X | Antibody designated as 60833 |
| C1.8 | Compound No. 8 of Group X | Antibody designated as 60833 |
| C1.9 | Compound No. 9 of Group X | Antibody designated as 60833 |
| C1.10 | Compound No. 10 of Group X | Antibody designated as 60833 |
| C1.11 | Compound No. 11 of Group X | Antibody designated as 60833 |
| C1.12 | Compound No. 12 of Group X | Antibody designated as 60833 |
| C1.13 | Compound No. 13 of Group X | Antibody designated as 60833 |
| C1.14 | Compound No. 14 of Group X | Antibody designated as 60833 |
| C1.15 | Compound No. 15 of Group X | Antibody designated as 60833 |
| C1.16 | Compound No. 16 of Group X | Antibody designated as 60833 |
| C1.17 | Compound No. 17 of Group X | Antibody designated as 60833 |
| C1.18 | Compound No. 18 of Group X | Antibody designated as 60833 |
| C1.19 | Compound No. 19 of Group X | Antibody designated as 60833 |
| C1.20 | Compound No. 20 of Group X | Antibody designated as 60833 |
| C1.21 | Compound No. 21 of Group X | Antibody designated as 60833 |
| C1.22 | Compound No. 22 of Group X | Antibody designated as 60833 |
| C1.23 | Compound No. 23 of Group X | Antibody designated as 60833 |
| C1.24 | Compound No. 24 of Group X | Antibody designated as 60833 |
| C1.25 | Compound No. 25 of Group X | Antibody designated as 60833 |

The combinations, compositions, kits, uses and methods according to the present invention are useful for the treatment and/or prevention of oncological and hyperproliferative disorders.

In certain embodiments, the hyperproliferative disorder is cancer.

Cancers are classified in two ways: by the type of tissue in which the cancer originates (histological type) and by primary site, or the location in the body, where the cancer first developed. The most common sites in which cancer develops include the skin, lung, breast, prostate, colon and rectum, cervix and uterus.

The combinations, compositions, kits, uses or methods according to the invention are useful in the treatment of a variety of cancer diseases, including, for example, but not limited to the following:
AIDS-related cancer such as Kaposi's sarcoma;
bone related cancer such as Ewing's family of tumours and osteosarcoma;
brain related cancer such as adult brain tumour, childhood brain stem glioma, childhood cerebellar astrocytoma, childhood cerebral astrocytoma/malignant glioma, childhood ependymoma, childhood medulloblastoma, childhood supratentorial primitive neuroectodermal tumours, childhood visual pathway and hypothalamic glioma and other childhood brain tumours;
breast cancer;
digestive/gastrointestinal related cancer such as anal cancer, extrahepatic bile duct cancer, gastrointestinal carcinoid tumour, gastrointestinal stroma tumour (GIST), cholangiocarcinoma, colon cancer, esophageal cancer, gallbladder cancer, adult primary liver cancer (hepatocellular carcinoma, hepatoblastoma) childhood liver cancer, pancreatic cancer, rectal cancer, small intestine cancer and stomach (gastric) cancer;
endocrine related cancer such as adrenocortical carcinoma, gastrointestinal carcinoid tumour, islet cell carcinoma (endocrine pancreas), parathyroid cancer, pheochromocytoma, pituitary tumour and thyroid cancer;
eye related cancer such as intraocular melanoma, and retinoblastoma;
genitourinary related cancer such as bladder cancer, kidney (renal cell) cancer, penile cancer, prostate cancer, transitional cell renal pelvis and ureter cancer, testicular cancer, urethral cancer, Wilms' tumour and other childhood kidney tumours;
germ cell related cancer such as childhood extracranial germ cell tumour, extragonadal germ cell tumour, ovarian germ cell tumour and testicular cancer;
gynecologic cancer such as cervical cancer, endometrial cancer, gestational trophoblastic tumour, ovarian epithelial cancer, ovarian germ cell tumour, ovarian low malignant potential tumour, uterine sarcoma, vaginal cancer and vulvar cancer;
head and neck related cancer such as hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer, parathyroid cancer and salivary gland cancer;
hematologic/blood related cancer such as leukemias, such as adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; and lymphomas, such as AIDS-related lymphoma, cutaneous T-cell lymphoma, adult Hodgkin's lymphoma, childhood Hodgkin's lymphoma, Hodgkin's lymphoma during pregnancy, mycosis fungoides, adult non-Hodgkin's lymphoma, childhood non-Hodgkin's lymphoma, non-Hodgkin's lymphoma during pregnancy, primary central nervous system lymphoma, Sezary syndrome, cutaneous T-cell lymphoma and Waldenström's macroglobulinemia and other hematologic/blood related cancer such as chronic myeloproliferative disorders, multiple myeloma/plasma cell neoplasm, myelodysplastic syndromes and myelodysplastic/myeloproliferative diseases;
musculoskeletalrelated cancer such as Ewing's family of tumours, osteosarcoma, malignant fibrous histiocytoma of bone, childhood rhabdomyosarcoma, adult soft tissue sarcoma, childhood soft tissue sarcoma and uterine sarcoma; hemangiosarcomas and angiosarcoma;
neurologicrelated cancer such as adult brain tumour, childhood brain tumour, brain stem glioma, cerebellar astrocytoma, cerebral astrocytoma/malignant glioma, ependmoma, medulloblastoma, supratentorial primitive neuroectodermal tumours, visual pathway and hypothalamic glioma and other brain tumours such as neuroblastoma, pituitary tumour and primary central nervous system lymphoma;
respiratory/thoracicrelated cancer such as non-small cell lung cancer, small cell lung cancer, malignant mesothelioma, thymoma and thymic carcinoma;
skin related cancer such as cutaneous T-cell lymphoma, Kaposi's sarcoma, melanoma, Merkel cell carcinoma and skin cancer;
Small blue round cell tumours.

In a further embodiment, the combinations, compositions, kits, uses or methods of the invention are beneficial in the treatment of cancers of the hematopoietic system including leukemias, lymphomas and myelomas, cancers of the gastrointestinal tract including esophageal, gastric, colorectal, pancreatic, liver and gall bladder and bile duct cancer; kidney, prostate and bladder cancer; gynecological cancers including breast, ovarian, cervical and endometrial cancer; skin and head and neck cancers including malignant melanomas; pediatric cancers like Wilms' tumour, neuroblastoma and Ewing'sarcoma; brain cancers like glioblastoma; sarcomas like osteosarcoma, soft tissue sarcoma, rhabdomyosarcoma, hemangiosarcoma; lung cancer including non-small cell lung cancer, mesothelioma and thyroid cancer.

In a further embodiment of the invention, the combinations, compositions, kits, uses or methods according to the invention are beneficial in the treatment of non-small cell lung cancer (NSCLC), including for example locally advanced or metastatic NSCLC (stage IIIB/IV). In this context, the combinations, compositions, kits, uses or methods of the invention may be further combined with or further involve platinum-based chemotherapy, such as e.g. paclitaxel/carboplatin or gemcitabine/cisplatin platinum doublet therapy.

In a further embodiment of the invention, the combinations, compositions, kits, uses or methods according to the invention are beneficial in the treatment of hepatocellular carcinoma, including for example locally advanced or hepatocellular carcinoma (stage III/IV). In this context combinations, compositions, kits, uses or methods of the invention may be further combined with or further involve sorafenib.

In another embodiment, the combinations, compositions, kits, uses and methods of the present invention refer to the treatment of subsets of cancer with addiction to MEK-signalling pathway, particularly such subsets of cancer with one or more mutations in the BRAF or RAS (e.g. KRAS and/or NRAS) gene.

In a further embodiment, the present invention relates to combinations, compositions, kits, uses or methods of this invention which are useful for treating cancers (tumors) comprising one or more of the following mutations:

BARF mutation in codons 464-469 and/or, particularly, in codon V600, such as e.g. a mutation selected from V600E, V600G, V600A and V600K, or a mutation selected from V600E, V600D, V600K and V600R, or a mutation selected from V600E, V600D and V600K, or a mutation selected from V600E, V600D, V600M, V600G, V600A, V600R and V600K; KRAS mutation in codon 12 (exon 1), codon 13 (exon 1) and/or codon 61 (exon 2), particularly in codons 12 and/or 13, such as e.g. a mutation selected from Gly12Asp, Gly12Val, Gly13Asp, Gly12Cys, Gly12Ser, Gly12Ala and Gly12Arg; or a mutation selected from 12D, 12V, 12C, 12A, 12S, 12R, 12F, 13D, 13C, 13R, 13S, 13A, 13V, 13I, 61H, 61L, 61R, 61K, 61E and 61P;

NRAS mutation in codons 12, 13 and/or 61, such as e.g. a mutation selected from p.G12D, p.G12S, p.G12C, p.G12V, p.G12A, p.G13D, p.G13R, p.G13C, p.G13A, p.Q61R, p.Q61K, p.Q61L, p.Q61H and p.Q61P.

In a further embodiment, the present invention relates to combinations, compositions, kits, uses or methods of this invention which are useful for treating cancers (tumors) comprising one or more of the following mutations:

BARF mutation in codons 464-469 and/or, particularly, in codon V600, such as e.g. a mutation selected from V600E, V600D, V600G, V600A, V600R, V600M and V600K.

In a further embodiment, the present invention relates to combinations, compositions, kits, uses or methods of this invention which are useful for treating cancers (tumors) comprising one or more of the following mutations:

KRAS mutation in codons 12, 13 and/or 61, particularly in codons 12 and/or 13, such as e.g. a mutation selected from Gly12Asp, Gly12Val, Gly13Asp, Gly12Cys, Gly12Ser, Gly12Ala and Gly12Arg; or a mutation selected from 12D, 12V, 12C, 12A, 12S, 12R, 12F, 13D, 13C, 13R, 13S, 13A, 13V, 13I, 61H, 61L, 61R, 61K, 61E and 61P.

In a further embodiment, the present invention relates to combinations, compositions, kits, uses or methods of this invention which are useful for treating cancers (tumors) comprising one or more of the following mutations:

NRAS mutation in codons 12, 13 and/or 61, such as e.g. a mutation selected from p.G12D, p.G12S, p.G12C, p.G12V, p.G12A, p.G13D, p.G13R, p.G13C, p.G13A, p.Q61R, p.Q61K, p.Q61L, p.Q61H and p.Q61P.

The combinations, compositions, kits, uses or methods according to the invention are also useful for treating BRAF and/or RAS mutated cancers. This offers a broad spectrum of indications and subpopulations. Examples of such cancer indications include the following:
Melanoma: high BRAF (˜43%) and NRAS (˜20%) mutation status,
CRC: substantial mutation rate (37% KRAS, 11% BRAF),
Pancreas: KRAS mutation status ˜70%, high unmet need,
NSCLC: moderate KRAS mutation rate (18%).

Further, the present invention relates to combinations, compositions, kits, uses or methods, each as described herein, useful in the treatment and/or prevention of cancer (particularly a cancer selected from those cancers described hereinabove or hereinbelow) in a patient whose cancer is addicted to MEK signalling pathway or in whose cancer MEK is activated, such as e.g. in a patient whose cancer has one or more mutations in BRAF or RAS (e.g. KRAS and/or NRAS), such as e.g. one or more of those mutations described herein.

Further, the present invention relates to combinations, compositions, kits, uses or methods, each as described herein, useful in the treatment and/or prevention of cancer (such as e.g. CRC, PAC, NSCLC or melanoma) in a patient whose cancer cells are characterized by a heterozygous or homozygous BRAF or RAS (e.g. KRAS and/or NRAS) mutational genotype.

Further, the present invention relates to combinations, compositions, kits, uses or methods, each as described herein, useful in the treatment and/or prevention of cancer (such as e.g. CRC, PAC, NSCLC or melanoma) in a patient whose cancer cells are characterized by a wildtype genotype.

In an embodiment, the present invention relates to combinations, compositions, kits, uses or methods according to this invention which are useful in the treatment and/or prevention of colorectal cancer (CRC), such as having one or more mutations in KRAS (e.g. in codons 12, 13 and/or 61, particularly in codons 12 and/or 13, such as a mutation selected from Gly12Asp, Gly12Val, Gly13Asp, Gly12Cys, Gly12Ser, Gly12Ala and Gly12Arg; or a mutation selected from 12D, 12V, 12C, 12A, 12S, 12R, 12F, 13D, 13C, 13R, 13S, 13A, 13V, 13I, 61H, 61L, 61R, 61K, 61E and 61P).

In an embodiment, the present invention relates to combinations, compositions, kits, uses or methods according to this invention which are useful in the treatment and/or prevention of colorectal cancer (CRC), such as having one or more mutations in BRAF (e.g. in codons 464 to 469 and/or, particularly in codon V600, such as a mutation selected from V600E, V600D, V600G, V600A, V600R, V600M and V600K).

In an embodiment, the present invention relates to combinations, compositions, kits, uses or methods according to this invention which are useful in the treatment and/or prevention of colorectal cancer (CRC), such as of wildtype genotype.

In an embodiment, the present invention relates to combinations, compositions, kits, uses or methods according to this invention which are useful in the treatment and/or prevention of pancreatic cancer (PAC), such as having one or more mutations in KRAS (e.g. in codons 12, 13 and/or 61, particularly in codons 12 and/or 13, such as a mutation selected from Gly12Asp, Gly12Val, Gly13Asp, Gly12Cys, Gly12Ser, Gly12Ala and Gly12Arg; or a mutation selected from 12D, 12V, 12C, 12A, 12S, 12R, 12F, 13D, 13C, 13R, 13S, 13A, 13V, 13I, 61H, 61L, 61R, 61K, 61E and 61P).

In an embodiment, the present invention relates to combinations, compositions, kits, uses or methods according to this invention which are useful in the treatment and/or prevention of pancreatic cancer (PAC), such as of wildtype genotype.

In an embodiment, the present invention relates to combinations, compositions, kits, uses or methods according to this invention which are useful in the treatment and/or prevention of malignant melanoma, such as having one or more mutations in BRAF (e.g. in codons 464 to 469 and/or, particularly in codon V600, such as a mutation selected from V600E, V600D, V600G, V600A, V600R, V600M and V600K).

In an embodiment, the present invention relates to combinations, compositions, kits, uses or methods according to this invention which are useful in the treatment and/or prevention of malignant melanoma, such as having one or more mutations in NRAS (e.g. in codons 12, 13 and/or 61, such as e.g. a mutation selected from p.G12D, p.G12S, p.G12C, p.G12V, p.G12A, p.G13D, p.G13R, p.G13C, p.G13A, p.Q61R, p.Q61K, p.Q61L, p.Q61H and p.Q61P).

In an embodiment, the present invention relates to combinations, compositions, kits, uses or methods according to this invention which are useful in the treatment and/or prevention of malignant melanoma, such as of wildtype genotype.

In an embodiment, the present invention relates to combinations, compositions, kits, uses or methods according to this invention which are useful in the treatment and/or prevention of non-small cell lung cancer (NSCLC), such as having one or more mutations in KRAS (e.g. in codons 12, 13 and/or 61, particularly in codons 12 and/or 13, such as a mutation selected from Gly12Asp, Gly12Val, Gly13Asp, Gly12Cys, Gly12Ser, Gly12Ala and Gly12Arg; or a mutation selected from 12D, 12V, 12C, 12A, 12S, 12R, 12F, 13D, 13C, 13R, 13S, 13A, 13V, 13I, 61H, 61L, 61R, 61K, 61E and 61P).

Accordingly, cancer types amenable for the therapy of this invention are each:

colorectal cancer (CRC), especially CRC harboring at least one KRAS mutation, such as one or more mutations in codon 12 (exon 1), codon 13 (exon 1) and/or codon 61 (exon 2) of KRAS oncogene, particularly in codons 12 and/or 13, such as e.g. one or more mutations selected from Gly12Asp, Gly12Val, Gly13Asp, Gly12Cys, Gly12Ser, Gly12Ala and Gly12Arg; or one or more mutations selected from 12D, 12V, 12C, 12A, 12S, 12R, 12F, 13D, 13C, 13R, 13S, 13A, 13V, 13I, 61H, 61L, 61R, 61K, 61E and 61P;
colorectal cancer (CRC), especially CRC harboring at least one BRAF mutation, such as one or more mutations in codons 464-469 and/or, particularly, in codon V600 of BRAF oncogene, such as e.g. one or more mutations selected from V600E, V600G, V600A and V600K, or one or more mutations selected from V600E, V600D, V600K and V600R, or one or more mutations selected from V600E, V600D and V600K, or one or more mutations selected from V600E, V600D, V600M, V600G, V600A, V600R and V600K;
pancreatic cancer (PAC), especially PAC harboring harboring KRAS wildtype or PAC harboring at least one KRAS mutation, such as one or more mutations in codon 12 (exon 1), codon 13 (exon 1) and/or codon 61 (exon 2) of KRAS oncogene, particularly in codons 12 and/or 13, such as e.g. one or more mutations selected from Gly12Asp, Gly12Val, Gly13Asp, Gly12Cys, Gly12Ser, Gly12Ala and Gly12Arg; or one or more mutations selected from 12D, 12V, 12C, 12A, 12S, 12R, 12F, 13D, 13C, 13R, 13S, 13A, 13V, 13I, 61H, 61L, 61R, 61K, 61E and 61P;
melanoma, especially melanoma harboring at least one BRAF mutation, such as one or more mutations in codons 464-469 and/or, particularly, in codon V600 of BRAF oncogene, such as e.g. one or more mutations selected from V600E, V600G, V600A and V600K, or one or more mutations selected from V600E, V600D, V600K and V600R, or one or more mutations selected from V600E, V600D and V600K, or one or more mutations selected from V600E, V600D, V600M, V600G, V600A, V600R and V600K;
melanoma, especially melanoma harboring at least one NRAS mutation, such as one or more mutations in codons 12, 13 and/or 61 of NRAS oncogene, such as e.g. one or more mutations selected from p.G12D, p.G12S, p.G12C, p.G12V, p.G12A, p.G13D, p.G13R, p.G13C, p.G13A, p.Q61R, p.Q61K, p.Q61L, p.Q61H and p.Q61P;
non-small-cell lung cancer (NSCLC), especially NSCLC harboring at least one KRAS mutation, such as one or more mutations in codon 12 (exon 1), codon 13 (exon 1) and/or codon 61 (exon 2) of KRAS oncogene, particularly in codons 12 and/or 13, such as e.g. one or more mutations selected from Gly12Asp, Gly12Val, Gly13Asp, Gly12Cys, Gly12Ser, Gly12Ala and Gly12Arg; or one or more mutations selected from 12D, 12V, 12C, 12A, 12S, 12R, 12F, 13D, 13C, 13R, 13S, 13A, 13V, 13I, 61H, 61L, 61R, 61K, 61E and 61P.

A particular cancer type amenable for the therapy of this invention is non-small-cell lung cancer (NSCLC), especially NSCLC harboring at least one KRAS mutation, such as one or more mutations in codon 12 (exon 1), codon 13 (exon 1) and/or codon 61 (exon 2) of KRAS oncogene, particularly in codons 12 and/or 13, such as e.g. one or more mutations selected from Gly12Asp, Gly12Val, Gly13Asp, Gly12Cys, Gly12Ser, Gly12Ala and Gly12Arg; or one or more mutations selected from 12D, 12V, 12C, 12A, 12S, 12R, 12F, 13D, 13C, 13R, 13S, 13A, 13V, 13I, 61H, 61L, 61R, 61K, 61E and 61P.

Depending on the disorder to be treated, the combinations, compositions, kits, uses or methods of the invention may be used on its own or in further combination with one or more additional therapeutic agents, in particular selected from DNA damaging agents or therapeutically active compounds that inhibit angiogenesis, signal transduction pathways or mitotic checkpoints in cancer cells.

Such a combined treatment may be given using free combinations of the substances or in the form of a fixed combination, including kit-of-parts. Pharmaceutical formulations of the additional combination partners may either be obtained commercially as pharmaceutical compositions or may be formulated by the skilled man using conventional methods.

In some embodiments, the additional therapeutic agent may be, without limitation, one or more inhibitors selected from the group of inhibitors of EGFR, VEGFR, HER2-neu, AuroraA, AuroraB, PLK and P13 kinase, FGFR, PDGFR, Raf, KSP or PDK1.

Further examples of additional therapeutic agents are inhibitors of CDK, Akt, src/bcr-abl, cKit, cMet/HGF, c-Myc, Flt3, HSP90, hedgehog antagonists, inhibitors of JAK/STAT, Mek, mTor, NFkappaB, the proteasome, Rho, an inhibitor of wnt signaling or an ubiquitination pathway inhibitor.

Examples for Aurora inhibitors are, without limitation, PHA-739358, AZD-1152, AT-9283, CYC-116, R-763, VX-667, MLN-8045, PF-3814735, SNS-314, VX-689, GSK-1070916, TTP-607, PHA-680626, MLN-8237 and ENMD-2076.

An example for a PLK inhibitor is GSK-461364.

Examples for raf inhibitors are BAY-73-4506 (also a VEGFR inhibitor), PLX-4032, RAF-265 (also a VEGFR inhibitor), sorafenib (also a VEGFR inhibitor), XL-281, and Nevavar (also an inhibitor of the VEGFR).

Examples for KSP inhibitors are ispinesib, ARRY-520, AZD-4877, CK-1122697, GSK-246053A, GSK-923295, MK-0731, SB-743921, LY-2523355, and EMD-534085.

Examples for a src and/or bcr-abl inhibitors are dasatinib, AZD-0530, bosutinib, XL-228 (also an IGF-1R inhibitor), nilotinib (also a PDGFR and cKit inhibitor), imatinib (also a cKit inhibitor), NS-187, KX2-391, AP-24534 (also an inhibitor of EGFR, FGFR, Tie2, Flt3), KM-80 and LS-104 (also an inhibitor of Flt3, Jak2).

An example for a PDK1 inhibitor is AR-12.

An example for a Rho inhibitor is BA-210.

Examples for P13 kinase inhibitors are PX-866, PX-867, BEZ-235 (also an mTor inhibitor), XL-147, XL-765 (also an mTor inhibitor), BGT-226, CDC-0941, GSK-1059615.

Examples for inhibitors of cMet or HGF are XL-184 (also an inhibitor of VEGFR, cKit, Flt3), PF-2341066, MK-2461, XL-880 (also an inhibitor of VEGFR), MGCD-265 (also an inhibitor of VEGFR, Ron, Tie2), SU-11274, PHA-665752, AMG-102, AV-299, ARQ-197, MetMAb, CGEN-241, BMS-777607, JNJ-38877605, PF-4217903, SGX-126, CEP-17940, AMG-458, INCB-028060, and E-7050.

An example for a c-Myc inhibitor is CX-3543.

Examples for Flt3 inhibitors are AC-220 (also an inhibitor of cKit and PDGFR), KW-2449, LS-104 (also an inhibitor of bcr-abl and Jak2), MC-2002, SB-1317, lestaurtinib (also an inhibitor of VEGFR, PDGFR, PKC), TG-101348 (also an inhibitor of JAK2), XL-999 (also an inhibitor of cKit, FGFR, PDGFR and VEGFR), sunitinib (also an inhibitor of PDGFR, VEGFR and cKit), and tandutinib (also an inhibitor of PDGFR, and cKit).

Examples for HSP90 inhibitors are, tanespimycin, alvespimycin, IPI-504, STA-9090, MEDI-561, AUY-922, CNF-2024, and SNX-5422.

Examples for JAK/STAT inhibitors are CYT-997 (also interacting with tubulin), TG-101348 (also an inhibitor of Flt3), and XL-019.

Examples for Mek inhibitors are ARRY-142886, AS-703026, PD-325901, AZD-8330, ARRY-704, RDEA-119, and XL-518.

Examples for mTor inhibitors are rapamycin, temsirolimus, deforolimus (which also acts as a VEGF inhibitor), everolimus (a VEGF inhibitor in addition), XL-765 (also a P13 kinase inhibitor), and BEZ-235 (also a P13 kinase inhibitor).

Examples for Akt inhibitors are perifosine, GSK-690693, RX-0201, and triciribine.

Examples for cKit inhibitors are masitinib, OSI-930 (also acts as a VEGFR inhibitor), AC-220 (also an inhibitor of Flt3 and PDGFR), tandutinib (also an inhibitor of Flt3 and PDGFR), axitinib (also an inhibitor of VEGFR and PDGFR), sunitinib (also an inhibitor of Flt3, PDGFR, VEGFR), and XL-820 (also acts as a VEGFR- and PDGFR inhibitor), imatinib (also a bcr-abl inhibitor), nilotinib (also an inhibitor of bcr-abl and PDGFR).

Examples for hedgehog antagonists are IPI-609, CUR-61414, GDC-0449, IPI-926, and XL-139.

Examples for CDK inhibitors are seliciclib, AT-7519, P-276, ZK-CDK (also inhibiting VEGFR2 and PDGFR), PD-332991, R-547, SNS-032, PHA-690509, PHA-848125, and SCH-727965.

Examples for proteasome inhibitors/NFkappaB pathway inhibitors are bortezomib, carfilzomib, NPI-0052, CEP-18770, MLN-2238, PR-047, PR-957, AVE-8680, and SPC-839.

An example for an ubiquitination pathway inhibitor is HBX-41108.

Examples for anti-angiogenic agents are inhibitors of the FGFR, PDGFR and VEGF(R), and thalidomides, such agents being selected from, without limitation, BIBF 1120, bevacizumab, motesanib, CDP-791, SU-14813, telatinib, KRN-951, ZK-CDK (also an inhibitor of CDK), ABT-869, BMS-690514, RAF-265, IMC-KDR, IMC-18F1, IMiDs, thalidomide, CC-4047, lenalidomide, ENMD-0995, IMC-D11, Ki-23057, brivanib, cediranib, 1B3, CP-868596, IMC-3G3, R-1530 (also an inhibitor of Flt3), sunitinib (also an inhibitor of cKit and Flt3), axitinib (also an inhibitor of cKit), lestaurtinib (also an inhibitor of Flt3 and PKC), vatalanib, tandutinib (also an inhibitor of Flt3 and cKit), pazopanib, PF-337210, aflibercept, E-7080, CHIR-258, sorafenib tosylate (also an inhibitor of Raf), vandetanib, CP-547632, OSI-930, AEE-788 (also an inhibitor of EGFR and Her2), BAY-57-9352 (also an inhibitor of Raf), BAY-73-4506 (also an inhibitor of Raf), XL-880 (also an inhibitor of cMet), XL-647 (also an inhibitor of EGFR and EphB4), XL-820 (also an inhibitor of cKit), nilotinib (also an inhibitor of cKit and brc-abl), CYT-116, PTC-299, BMS-584622, CEP-11981, dovitinib, CY-2401401, and ENMD-2976.

The additional therapeutic agent may also be selected from EGFR inhibitors, it may be a small molecule EGFR inhibitor or an anti-EGFR antibody. Examples for anti-EGFR antibodies, without limitation, are cetuximab, panitumumab, nimotuzumab, zalutumumab; examples for small molecule EGFR inhibitors are gefitinib, erlotinib and vandetanib (also an inhibitor of the VEGFR). Another example for an EGFR modulator is the EGF fusion toxin.

Further EGFR and/or Her2 inhibitors useful for further combination are BIBW 2992, lapatinib, trastuzumab, pertuzumab, XL-647, neratinib, BMS-599626 ARRY-334543, AV-412, mAB-806, BMS-690514, JNJ-26483327, AEE-788 (also an inhibitor of VEGFR), AZD-8931, ARRY-380 ARRY-333786, IMC-11F8, Zemab, TAK-285, AZD-4769.

Other agents that may be advantageously combined within the therapy of the invention are tositumumab and ibritumomab tiuxetan (two radiolabelled anti-CD20 antibodies); ofatumumab, rituximab, LY-2469298, ocrelizumab, TRU-015, PRO-131921, FBT-A05, veltuzumab, R-7159 (CD20 inhibitors), alemtuzumab (an anti-CD52 antibody), denosumab, (an osteoclast differentiation factor ligand inhibitor), galiximab (a CD80 antagonist), zanolimumab (a CD4 antagonist), SGN40 (a CD40 ligand receptor modulator), XmAb-5485, Chi Lob 7/4, lucatumumab, CP-870893 (CD40 inhibitors), CAT-8015, epratuzumab, Y90-epratuzumab, inotuzumab ozogamicin (CD22 inhibitors), lumiliximab (a CD23 inhibitor), TRU-016 (a CD37 inhibitor), MDX-1342, SAR-3419, MT-103 (CD19 inhibitors), or mapatumumab, tigatuzumab, lexatumumab, Apomab, AMG-951 and AMG-655 (TRAIL receptor modulators).

Other chemotherapeutic drugs that may be used in combination within the therapy of the present invention are selected from, but not limited to hormones, hormonal analogues and antihormonals (e.g. tamoxifen, toremifene, raloxifene, fulvestrant, megestrol acetate, flutamide, nilutamide, bicalutamide, cyproterone acetate, finasteride, buserelin acetate, fludrocortinsone, fluoxymesterone, medroxyprogesterone, octreotide, arzoxifene, pasireotide, vapreotide), aromatase inhibitors (e.g. anastrozole, letrozole, liarozole, exemestane, atamestane, formestane), LHRH agonists and antagonists (e.g. goserelin acetate, leuprolide, abarelix, cetrorelix, deslorelin, histrelin, triptorelin), antimetabolites (e.g. antifolates like methotrexate, pemetrexed, pyrimidine analogues like 5-fluorouracil, capecitabine, decitabine, nelarabine, and gemcitabine, purine and adenosine analogues such as mercaptopurine thioguanine, cladribine and pentostatin, cytarabine, fludarabine); antitumour antibiotics (e.g. anthracyclines like doxorubicin, daunorubicin, epirubicin and idarubicin, mitomycin-C, bleomycin dactinomycin, plicamycin, mitoxantrone, pixantrone, streptozocin); platinum derivatives (e.g. cisplatin, oxaliplatin, carboplatin, lobaplatin, satraplatin); alkylating agents (e.g. estramustine, meclorethamine, melphalan, chlorambucil, busulphan, dacarbazine, cyclophosphamide, ifosfamide, hydroxyurea, temozolomide, nitrosoureas such as carmustine and lomustine, thiotepa); antimitotic agents (e.g. vinca alkaloids like vinblastine, vindesine, vinorelbine, vinflunine and vincristine; and taxanes like paclitaxel, docetaxel and their formulations, larotaxel; simotaxel, and epothilones like ixabepilone, patupilone, ZK-EPO); topoisomerase inhibitors (e.g. epipodophyllotoxins like etoposide and etopophos, teniposide, amsacrine, topotecan, irinotecan) and miscellaneous chemotherapeutics such as amifostine, anagrelide, interferone alpha, procarbazine, mitotane, and porfimer, bexarotene, celecoxib.

In an embodiment, the combination therapy of this invention may be further combined with platinum-based chemotherapy, for example with paclitaxel/carboplatin or gemcitabine/cisplatin platinum doublet therapy. Such regimen can be used e.g. in the treatment of NSCLC or in the treatment of hepatocellular carcinoma.

Other agents which may be combined within the therapy of the invention are agents that target the IGF-1R. Such agents include antibodies that bind to IGF-1R (e.g. CP-751871, AMG-479, IMC-Al2, MK-0646, AVE-1642, R-1507, BUB-022, SCH-717454, rhu Mab IGFR and novel chemical entities that target the kinase domain of the IGF1-R (e.g. OSI-906 or BMS-554417, XL-228, BMS-754807).

The combination therapy of this invention may also be further combined with other therapies including surgery, radiotherapy, radio-immunetherapy, endocrine therapy, biologic response modifiers, hyperthermia and cryotherapy and agents to attenuate any adverse effect, e.g. antiemetics.

The therapeutic applicability of the combination therapy according to this invention may include first line, second line, third line or further lines treatment of patients. The cancer may be metastatic, recurrent, relapsed, resistant or refractory to one or more anti-cancer treatments. Thus, the patients may be treatment naïve, or may have received one or more previous anti-cancer therapies, which have not completely cured the disease. Patients with relapse and/or with resistance to one or more anti-cancer agents (e.g. the single components of the combination, or standard chemotherapeutics) are also amenable for combined treatment according to this invention, e.g. for second or third line treatment cycles (optionally in further combination with one or more other anti-cancer agents), e.g. as add-on combination or as replacement treatment.

Accordingly, some of the disclosed combination therapies of this invention are effective at treating subjects whose cancer has relapsed, or whose cancer has become drug resistant or multi-drug resistant, or whose cancer has failed one, two or more lines of mono- or combination therapy with one or more anti-cancer agents (e.g. the single components of the combination, or standard chemotherapeutics).

A cancer which initially responded to an anti-cancer drug can relapse and it becomes resistant to the anti-cancer drug when the anti-cancer drug is no longer effective in treating the subject with the cancer, e.g. despite the administration of increased dosages of the anti-cancer drug. Cancers that have developed resistance to two or more anti-cancer drugs are said to be multi-drug resistant.

Accordingly, in some methods of combination treatment of this invention, treatment with a combination according to this invention administered secondly or thirdly is begun if the patient has resistance or develops resistance to one or more agents administered initially or previously. The patient may receive only a single course of treatment with each agent or multiple courses with one, two or more agents.

In certain instances, combination therapy according to this invention may hence include initial or add-on combination, replacement or maintenance treatment.

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

Further embodiments, features and advantages of the present invention may become apparent from the following examples. The following examples serve to illustrate, by way of example, the principles of the invention without restricting it.

### EXAMPLES

### 1. Aurora B Kinase Assays

Radioactive Kinase Assay Using a wild Type (wt)-Xenopus Iaevis Aurora B/INCENP Complex:

Protein Expression:

Preparation of the wild type (wt)-Xenopus/aevis Aurora B⁶⁰⁻³⁶¹/INCENP⁷⁹⁰⁻⁸⁴⁷ complex was performed essentially as described in Sessa et al. 2005. The ATP-K_{M} value of the complex is 61 μM. The kinase assays are run in the presence of 100 μM ATP using 10 μM of a substrate peptide. pAUB-IN847 was used to transform the E. coli strain BL21(DE3) containing the pUBS520 helper plasmid. Both proteins and their mutants are expressed and purified under essentially identical conditions. Protein expression is induced with 0.3 mM IPTG at an OD₆₀₀ of 0.45-0.7. Expression is then continued for about 12-16 hours at 23-25° C. with agitation. Bacterial cells are harvested by centrifugation at 4000 rpm×15 min in a Beckman JLA 8.1 rotor, and the pellets resuspended in lysis buffer (50 mM Tris HCl pH 7.6, 300 mM NaCl, 1 mM DTT, 1 mM EDTA, 5% glycerol, Roche Complete protease inhibitor tablets). 20-30 ml lysis buffer are used per liter of E. coli culture. Cells are lysed by sonication, and the lysates cleared by centrifugation at 12000 rpm for 45-60 min on a JA20 rotor. The supernatants are incubated with 300 μl of GST Sepharose Fast Flow (Amersham Biosciences) per liter of bacterial culture. The resin is first washed with PBS buffer and finally equilibrated with lysis buffer. After a 4-5 hour agitation at 4° C., the beads are washed with 30 volumes of lysis buffer, and then equilibrated with 30 volumes of cleavage buffer (50 mM Tris pH 7.6, 150 mM NaCl, 1 mM DTT, 1 mM EDTA). To cleave the GST from Aurora B, 10 units of Prescission protease (Amersham Biosciences) per milligram of substrate are added and the incubation is protracted for 16 hours at 4° C. The supernatant, which contains the cleaved product, is collected and loaded onto a 6 ml Resource Q column (Amersham Biosciences) equilibrated with Ion Exchange buffer (50 mM Tris pH 7.6, 150 mM NaCl, 1 mM DTT, 1 mM EDTA). The Aurora B/INCENP complex is collected in the flow through of the column. The flow-through of the Resource Q column is concentrated and loaded onto a Superdex 200 size-exclusion chromatography (SEC) column equilibrated with SEC buffer (Tris HCl 10 mM pH 7.6, NaCl 150 mM, DTT 1 mM, EDTA 1 mM). Fractions containing Aurora-B/INCENP are collected and concentrated using Vivaspin concentrators (MW cutoff 3-5 K) to a final concentration of 12 mg/ml. The final yield is about 1-2 mg of pure complex per liter of bacteria. Purified (wt)-Xenopus laevis Aurora B⁶³⁻³⁶¹/INCENP⁷⁹⁰⁻⁸⁴⁷ complex was stored at −80° C. in desalting buffer (50 mM Tris/Cl pH 8.0, 150 mM NaCl, 0.1 mM EDTA, 0.03% Brij-35, 10% glycerol, 1 mM DTT).

Assay Conditions:

Enzyme activity was assayed in the presence or absence of serial inhibitor dilutions. For the kinase assay (reaction volume 50 μl/well), 96-well PP-Microplates (Greiner, 655 201) were used. To 10 μl compound in 25% DMSO were added: 30 μl PROTEIN-MIX (166 μM ATP, kinase buffer [50 mM Tris/HCl pH 7.5, 25 mM MgCl₂, 25 mM NaCl], 10 ng wt-Aurora-B60-361/INCENP790-847) followed by an 15 min incubation at room temperature (agitating, 350 rpm). To this, 10 μl PEPTIDE-MIX (2× kinase buffer, 5 mM NaF, 5 mM DTT, 1 μCi ³³P-ATP, 50 μM peptide (Biotin-LRRWSLGLRRWSLGLRRW SLGLRRWSLG) was added. The mixture was incubated for 60 min at room temperature (agitating, 350 rpm), followed by addition of 180 μl 6.4% TCA (final concentration: 5%) to stop the reaction. Subsequently, a Multiscreen filtration plate (Millipore, MAI P N0B 10) was equilibrated with 100 μl 70% ethanol and 1% TCA prior to addition of the stopped kinase reaction. Following 5 washes with 180 μl 1% TCA, the lower part of the plate was dried. 25 μl scintillation cocktail (Microscint, High Efficiency LSC-Cocktail, Packard, 6013611) was added and the incorporated gamma phosphate was measured in a suitable scintillation counter.

Data Analysis:

Inhibitor concentrations were transformed to logarithmic values and the raw data were normalized. These normalized values were used to calculate the IC₅₀ values. Data was fitted by iterative calculation using a sigmoidal curve analysis program (Graph Pad Prism version 3.0) with variable Hill slope. Each microtiter plate contained internal controls, such as blank, maximum reaction and historical reference compound.

Analysis of Histone H3 Phosphorylation in NCl-H460 cells:

NCl-H460 cells were plated in 96 well flat bottom Falcon plates at a cell density of 4000 cells/well. On the next day, cells were synchronized by treating them for 16 hrs with 300 nM BIVC0030BS. This CDK1 inhibitor arrests cells in G2. The cells were released from the inhibitory G2 block by washing once with medium. The synchronous entry into mitosis results in a high percentage (70-80%) of mitotic cells after 60 min. Fresh medium and compounds were added to the wells, each drug concentration in duplicates. The final volume per well was 200 μl and the final concentration of the test compounds covered the range between 10 μM and 5 nM. The final DMSO concentration was 0.1%. Cells were incubated at 37° C. and 5% CO₂ in a humidified atmosphere for exactly 60 minutes. The medium was aspirated and the cells were fixed and permeabilized with 100 μl warm 4% formaldehyde solution containing Triton X-100 (1:200) for 10 min at RT. After washing twice with blocking buffer (0.3% BSA/PBS), 50 μl solution of polyclonal antibody anti-phospho H3 (Ser28) diluted 1:500 was added for 1 hr at RT. After washing twice with blocking buffer, cells were incubated with 50 μl goat-anti rabbit F(ab)₂ fragment Alexa Fluor 594 (1:2000)+DAPI (final concentration 300 nM) for 1 hr at RT in the dark. The plates were washed, 200 μl PBS were added, the plates sealed with black foil and analyzed in a Cellomics ArrayScan applying the Cell Cycle BioApplication program. The data generated in the assay were analyzed by the program PRISM (GraphPad Inc.). The inhibitor concentrations were transformed to logarithmic values and EC₅₀ was calculated by a nonlinear regression curve fit (sigmoidal dose-response (variable slope)).

### 2. MEK Kinase Assays

MEK inhibitory activity of a compound is measured using the Z′-LYTE™ kinase assay of Invitrogen.

The T-LYTE® biochemical assay employs a fluorescence-based, coupled-enzyme format and is based on the differential sensitivity of phosphorylated and non-phosphorylated peptides to proteolytic cleavage. The peptide substrate is labeled with two fluorophores—one at each end—that make up a FRET pair.

In the primary reaction, the kinase transfers the gamma-phosphate of ATP to a single tyrosine, serine or threonine residue in a synthetic FRET-peptide. In the secondary reaction, a site-specific protease recognizes and cleaves non-phosphorylated FRET-peptides. Phosphorylation of FRET-peptides suppresses cleavage by the Development Reagent. Cleavage disrupts FRET between the donor (i.e. coumarin) and acceptor (i.e., fluorescein) fluorophores on the FRET-peptide, whereas uncleaved, phosphorylated FRET-peptides maintain FRET. A ratiometric method, which calculates the ratio (the Emission Ratio) of donor emission to acceptor emission after excitation of the donor fluorophore at 400 nm, is used to quantitate reaction progress, as shown in the equation as follows:

Emission Ratio=Coumarin emission (445 nM)/Fluorescein Emission (520 nM).

Both cleaved and uncleaved FRET-peptides contribute to the fluorescence signals and therefore to the Emission Ratio. The extent of phosphorylation of the FRET-peptide can be calculated from the Emission Ratio. The Emission Ratio will remain low if the FRET-peptide is phosphorylated (i.e., no kinase inhibition) and will be high if the FRET-peptide is non-phosphorylated (i.e., kinase inhibition).

The Test Compounds are screened in 1% DMSO (final) in the well. For 10 point titrations, 3-fold serial dilutions are conducted from the starting concentration (1 μM). All Peptide/Kinase Mixtures are diluted to a 2× working concentration in the appropriate Kinase Buffer.

All ATP Solutions are diluted to a 4× working concentration in Kinase Buffer (50 mM HEPES pH 7.5, 0.01% BRIJ-35, 10 mM MgCl2, 1 mM EGTA).

ATP Km apparent is previously determined using a Z′-LYTE® assay.

### Assay Protocol:

1. 2.5 μL—4× Test Compound or 100 nL 100× plus 2.4 μL kinase buffer

2. 5 μL—2× Peptide/Kinase Mixture

3. 2.5 μL—4×ATP Solution

4. 30-second plate shake

5. 60-minute Kinase Reaction incubation at room temperature

6. 5 μL—Development Reagent Solution

7. 30-second plate shake

8. 60-minute Development Reaction incubation at room temperature

9. Read on fluorescence plate reader and analyze the data

### MAP2K1 (MEK1) Specific Assay Conditions—Cascade Format:

The 2×MAP2K1 (MEK1)/inactive MAPK1 (ERK2)/Ser/Thr 03 mixture is prepared in 50 mM HEPES pH 7.5, 0.01% BRIJ-35, 10 mM MgCl2, 1 mM EGTA. The final 10 μL Kinase Reaction consists of 1.29-5.18 ng MAP2K1 (MEK1), 105 ng inactive MAPK1 (ERK2), and 2 μM Ser/Thr 03 in 50 mM HEPES pH 7.5, 0.01% BRIJ-35, 10 mM MgCl2, 1 mM EGTA. After the 1 hour Kinase Reaction incubation, 5 μL of a 1:1024 dilution of Development Reagent A is added.

### MAP2K2 (MEK2) Specific Assay Conditions—Cascade Format:

The 2×MAP2K2 (MEK2)/inactive MAPK1 (ERK2)/Ser/Thr 03 mixture is prepared in 50 mM HEPES pH 7.5, 0.01% BRIJ-35, 10 mM MgCl2, 1 mM EGTA. The final 10 μL Kinase Reaction consists of 1.13-4.5 ng MAP2K2 (MEK2), 105 ng inactive MAPK1 (ERK2), and 2 μM Ser/Thr 03 in 50 mM HEPES pH 7.5, 0.01% BRIJ-35, 10 mM MgCl2, 1 mM EGTA. After the 1 hour Kinase Reaction incubation, 5 μL of a 1:1024 dilution of Development Reagent A is added.

### Z′-LYTE® Assay Controls:

0% Phosphorylation Control (100% Inhibition Control):

The maximum Emission Ratio is established by the 0% Phosphorylation Control (100% Inhibition Control), which contains no ATP and therefore exhibits no kinase activity. This control yields 100% cleaved peptide in the Development Reaction. 100% Phosphorylation Control:

The 100% Phosphorylation Control, which consists of a synthetically phosphorylated peptide of the same sequence as the peptide substrate, is designed to allow for the calculation of percent phosphorylation.

This control yields a very low percentage of cleaved peptide in the Development Reaction. The 0% Phosphorylation and 100% Phosphorylation Controls allow one to calculate the percent Phosphorylation achieved in a specific reaction well. Control wells do not include any kinase inhibitors.

0% Inhibition Control:

The minimum Emission Ratio in a screen is established by the 0% Inhibition Control, which contains active kinase. This control is designed to produce a 10-70% phosphorylated peptide in the Kinase Reaction.

A known inhibitor (staurosporine IC50 MEK1/MEK2 14.7 nM/15.2 nM at 100 μM ATP) control standard curve, 10 point titration, is run for each individual kinase on the same plate as the kinase to ensure the kinase is inhibited within an expected IC50 range previously determined.

### Development Reaction Interference:

The Development Reaction Interference is established by comparing the Test Compound Control wells that do not contain ATP versus the 0% Phosphorylation Control (which does not contain the Test Compound). The expected value for a non-interfering compound should be 100%. Any value outside of 90% to 110% is flagged.

### Test Compound Fluorescence Interference:

The Test Compound Fluorescence Interference is determined by comparing the Test Compound Control wells that do not contain the Kinase/Peptide Mixture (zero peptide control) versus the 0% Inhibition Control. The expected value for a non-fluorescence compound should be 0%. Any value >20% is flagged.

As graphing software XLfit from IDBS is used. The dose response curve is curve fit to model number 205 (sigmoidal dose-response model). If the bottom of the curve does not fit between −20% & 20% inhibition, it is set to 0% inhibition. If the top of the curve does not fit between 70% and 130% inhibition, it is set to 100% inhibition.

### Analysis of Phosphorylation of ERK in SK-MEL-28 Cells:

Fast actived cell-based ELISA (FACE) SK-MEL-28 p-ERK:

### Cell Culture:

SK-MEL28 cells (human melanoma) are grown in T75 flascs using MEM medium supplemented with 10% fetal calf serum, 2% Na bicarbonate, 1% Na pyruvate solution, 1% NEAA 100× and 2 mM L-Glutamine. Cultures are incubated at 37° C. and 5% CO2 in a humidified atmosphere, with medium change or subcultivation 2 times a week

### Assay Conditions:

7,500 cells per well/90 μl medium are plated in 96 well plates (Flat bottom, Costar #3598). At the next day compounds (Stock: 10 mM in 100% DMSO) are diluted in medium (stock solution) or serially diluted in medium plus 10% DMSO (all other dilution steps). 10 μl of diluted compound is added per well, the final concentration of DMSO is 1%. The concentration of the test compounds covers usually the range between 10 micromolar and 2.4 nanomolar minimum. Cells are incubated at 37° C. and 5% CO2 in a humidified atmosphere for 2 hours.

The supernatant is removed. Cells are fixed with 150 μl 4% formaldehyde in PBS for 20 minutes at room temperature.

The cell layer is washed 5 times with 200 μl 0.1% Triton X-100 in PBS for 5 minutes each, followed by a 90 minutes incubation with blocking buffer (5% non-fat dry milk in TBS-T). Blocking buffer is replaced by 50 μl/well of the 1st antibody [monoclonal anti-MAP Kinase diphosphorylated Erk-1&2 (Sigma, #M8159); 1:500 Verd.] and incubated over night at 4° C. The cell layer is washed 5 times with 200 μl 0.1% Triton X-100 in PBS for 5 minutes each. The cell layer is incubated with 50 μl/well of the second antibody [polyclonal rabbit-anti-Mouse HRPO coupled, (Dako, #P0161); 1:1000 dilution in blocking buffer] for 1 hour. The cell layer is washed 5 times with 200 μl 0.1% Tween20 in PBS for 5 minutes each. Peroxidase staining is performed by adding 100 μl/well of the staining solution (TMB Peroxidase Substrate Solution; Bender MedSystems #BMS406), for 5-30 minutes in the dark. The reaction is stopped by adding 100 μl/well of 1 M phosphoric acid.

The stain is measured at 450 nm with a Multilabel Reader (Wallac Victor 2). Data are fitted by iterative calculation using a sigmoidal curve analysis program (Prism version 3.0, Graph PAD) with variable hill slope (FIFTY version 2).

### In Vivo Efficacy

The in vivo efficacy of a dual Aurora kinase/MEK inhibitor according to this invention is assessed in standard human tumor models displaying various oncogenome signatures in nude mice: For example, xenografts derived from HCT116 (K-RAS^{G13G/D} and PIK3CA^{H1047H/R} mutant), and Colo205 (B-RAF^{V600E} mutant) colon carcinomas, the NCl-H460 (K-RAS^{Q61H} and PIK3CA^{E545K/E} mutant) and Calu-6 (K-RAS^{Q61K} and TP53^{R196*} mutant) non-small-cell lung carcinoma, the BxPC-3 (TP53^{Y220C} mutant) pancreatic carcinoma or the melanoma A-375 (B-RAE^{V600E} mutant) cell lines are established models for the preclinical evaluation of oncology compounds. Tumor cells are injected subcutaneously (s.c.) into the right flank of nude mice. In addition, the efficacy of a dual MEK/Aurora B kinase inhibitor according to this invention is assessed in a nude mouse xenograft model of human colon carcinoma with MDR1 overexpression (C×B1 tumor transplants also display K-RAS^{G13D} and TP53^{R175H and P72R} mutations). Mice bearing established tumors with an average volume of 50-100 mm³ are randomized into treatment and control groups. The maximum tolerated dose (MTD) is determined in tolerability tests in tumor-free nude mice before the xenograft experiment. Preferably, the dual Aurora kinase/MEK inhibitor according to this invention is administered orally (p.o.).

Efficacious treatment with the respective compound is characterised by growth delay upon treatment when used at its respective MTD. Preferably, prolonged treatment induces tumor regressions in the treated animals. Pharmacodynamic inhibition of MEK can be monitored in vivo by determining the phosphorylation state of ERK/MAPK, a direct substrate of MEK. Immunohistochemical analyses confirms target inhibition displaying a significant reduction (>50%) in pERK tumor levels in treated animals compared to vehicle-treated controls.

### Experimental Procedure of Combination Use for Cancer Cell Proliferation Inhibition:

Cells are grown in RPMI1640, 5% FBS, 2 mM L-alanyl-L-glutamine, 1 mM Na pyruvate or a special medium in a humidified atmosphere of 5% CO₂ at 37° C. Cells are seeded into in flat bottom 96 well microtiter plates and incubated in a humidified atmosphere of 5% CO₂ at 37° C. for 24 hours.

Cells are left unstimulated or stimulated with 20 ng/ml of human IGF-1. Compounds are added 30 minutes post cell stimulation. At the same time, a “time zero” untreated cell plate is fixed. Compounds are serially diluted 5-fold from the highest test concentration (1 or 2 μM) and assayed over 5 concentrations in duplicates. The concentration of the solvent DMSO in the final culture is 0.1%. After a 120 hour incubation period, cells are fixed and stained with fluorescent nuclear dye to allow visualization of nuclei (CyQuant Direct Cell Proliferation Assay, Invitrogen Cat. No. C35012). Total fluorescence intensity of each well is measured using an Envision platform with excitation at 480 nm, emission detection at 535 nm. The assay signal correlates to the number of nuclei and thus, by definition, to the number of cells in the culture well (“cell count”).

The cell proliferation assay output for control cells after 120 hours of incubation, corresponding to 100% cell proliferation, is taken as the reference cell count for all subsequent calculations. Relative cell growth inhibition (CGI %) in compound-treated cultures is calculated according to the following formula:

$$\% C G {I}^{120 h} = \{ \begin{matrix}{S}_{t}^{120} \geq {S}_{c}^{0} : [ 1 - \frac{{S}_{t}^{120 h} - {S}_{c}^{0 h}}{{S}_{c}^{120 h} - {S}_{c}^{0 h}} ] \times 100 \% \\ {S}_{t}^{120} < {S}_{c}^{0} : [ 1 - \frac{{S}_{t}^{120 h} - {S}_{c}^{0 h}}{{S}_{c}^{0 h}} ] \times 100 \%\end{matrix}$$

Further, the Bliss additivism model was used to identify synergies. This model predicts the combined response C for two single compounds with effects A and B:

C=A+B−A*B

The excess inhibition over the predicted Bliss additivism model was calculated by subtracting the predicted Bliss effect from the experimentally observed inhibition at each pair of concentrations.

Combinations were rated on the base of the excess over the Bliss additivism calculated for all pair of concentrations as follow:

− no effect
−/+ less than additive
+ less than additive/additive
++ more than additive/synergistic

Results: The following table summarizes the rating of combination effects obtained for an exemplary combination of a certain dual Aurora kinase/MEK inhibitor selected from the Group X as defined herein (dual MEK/AKI #6) and a certain anti-IGF antibody designated as 60833 herein (or control human IgG, respectively) on NSCLC cell types:

| | | Combo | |
|---|---|---|---|
| | | dual MEK/AKI #6 | |
| | | anti-IGF mAb | Control human |
| NSCLC | Mutation | 60833 | IgG |
| NCI-H460 | KRAS, PIK3CA | − | − |
| Calu-6 | KRAS | + | − |
| NCI-H292 | wt | + | − |
| NCI-H2122 | KRAS | ++ | −/+ |
| NCI-H441 | KRAS | − | − |

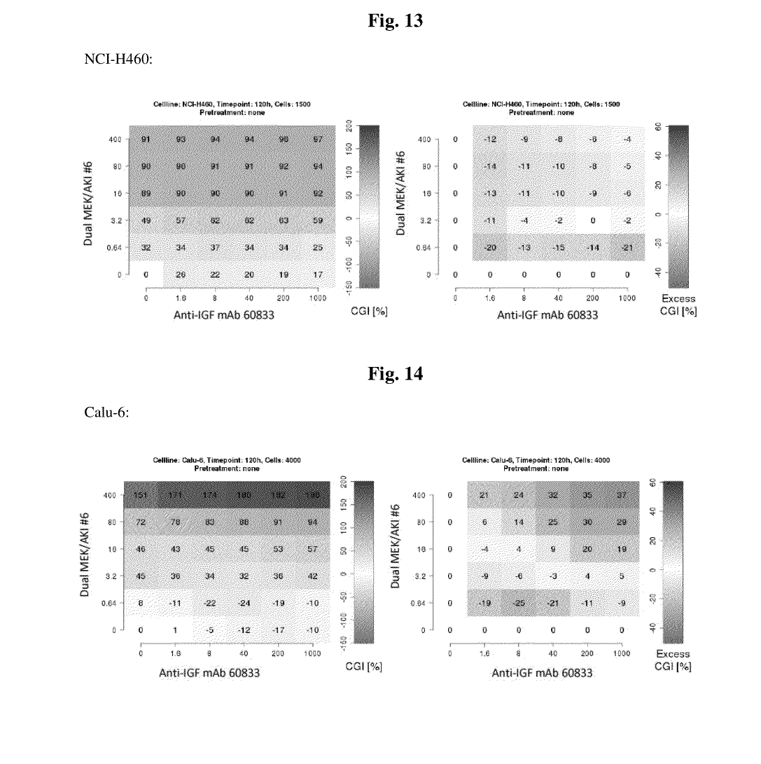

For example, the values of relative cell growth inhibition (CGI %) and excess inhibition over the predicted Bliss additivism for rating of combination effects obtained by using the above exemplary combo dual MEK/AKI+anti-IGF mAb of the present invention (embodiment C1.6 of Table i) in the above-described experimental procedure (cell lines NCl-H460 and Calu-6 shown, for example) are shown in FIGS. 13 and 14.

The data support that a combination according to the present invention is useful for the herein-described therapeutic purposes, such as e.g. for treating NSCLC.

What is claimed is:

1. A method of treating or preventing an oncological or hyperproliferative disease comprising administering to a patient in need thereof a therapeutically effective amount of a dual Aurora kinase/MEK inhibitor and an anti-IGF antibody.

2. The method according to claim 1, wherein the dual Aurora kinase/MEK inhibitor is selected from
1) N-ethyl-3-[3-[[4-(4-methylpiperazin-1-yl)anilino]-phenylmethylidene]-2-oxo-1H-indol-6-yl]prop-2-ynamide,
2) N-(2,2-difluoroethyl)-3-[3-[[4-(dimethylaminomethyl)anilino]-phenylmethylidene]-2-oxo-1H-indol-6-yl]prop-2-ynamide,
3) N-(2,2-difluoroethyl)-3-[2-oxo-3-[phenyl-[4-(pyrrolidin-1-ylmethyl)anilino]methylidene]-1H-indol-6-yl]prop-2-ynamide,
4) N-(2-fluoroethyl)-3-[2-oxo-3-[phenyl-[4-(pyrrolidin-1-ylmethyl)anilino]methylidene]-1H-indol-6-yl]prop-2-ynamide,
5) N-ethyl-3-[2-oxo-3-[phenyl-[4-(pyrrolidin-1-ylmethyl)anilino]methylidene]-1H-indol-6-yl]prop-2-ynamide,
6) 3-[3-[[4-(dimethylaminomethyl)anilino]-phenylmethylidene]-2-oxo-1H-indol-6-yl]-N-ethylprop-2-ynamide,
7) N-cyclobutyl-3-[3-[[4-(4-methylpiperazin-1-yl)anilino]-phenylmethylidene]-2-oxo-1H-indol-6-yl]prop-2-ynamide,
8) N-cyclopropyl-3-[3-[[4-(dimethylaminomethyl)anilino]-phenylmethylidene]-2-oxo-1H-indol-6-yl]prop-2-ynamide,
9) 3-[3-[[4-(dimethylaminomethyl)anilino]-phenylmethylidene]-2-oxo-1H-indol-6-yl]-N-phenylprop-2-ynamide,
10) N-cyclopentyl-3-[3-[[4-(dimethylaminomethyl)anilino]-phenylmethylidene]-2-oxo-1H-indol-6-yl]prop-2-ynamide,
11) N-cyclopentyl-3-[3-[[4-(4-methylpiperazin-1-yl)anilino]-phenylmethylidene]-2-oxo-1H-indol-6-yl]prop-2-ynamide,
12) N-cyclobutyl-3-[3-[[4-(dimethylaminomethyl)anilino]-phenylmethylidene]-2-oxo-1H-indol-6-yl]prop-2-ynamide,
13) 3-[3-[[4-(dimethylaminomethyl)anilino]-phenylmethylidene]-2-oxo-1H-indol-6-yl]-N-(2-hydroxyethyl)prop-2-ynamide,
14) 3-[3-[[4-(dimethylaminomethyl)anilino]-phenylmethylidene]-2-oxo-1H-indol-6-yl]-N-propan-2-ylprop-2-ynamide,
15) 3-[2-oxo-3-[phenyl-[4-(pyrrolidin-1-ylmethyl)anilino]methylidene]-1H-indol-6-yl]-N-propan-2-ylprop-2-ynamide,
16) N-(2-hydroxyethyl)-3-[2-oxo-3-[phenyl-[4-(pyrrolidin-1-ylmethyl)anilino]methylidene]1H-indol-6-yl]prop-2-ynamide,
17) N-(2-fluorophenyl)-3-[2-oxo-3-[phenyl-[4-(pyrrolidin-1-ylmethyl)anilino]methylidene]-1H-indol-6-yl]prop-2-ynamide,
18) 3-[3-[[4-(dimethylaminomethyl)anilino]-phenylmethylidene]-2-oxo-1H-indol-6-yl]-N-[(2S)-1-hydroxypropan-2-yl]prop-2-ynamide,
19) N-[(2S)-1-hydroxypropan-2-yl]-3-[2-oxo-3-[phenyl-[4-(pyrrolidin-1-ylmethyl)anilino]methylidene]-1H-indol-6-yl]prop-2-ynamide,
20) N-[(2R)-butan-2-yl]-3-[2-oxo-3-[phenyl-[4-(pyrrolidin-1-ylmethyl)anilino]methylidene]-1H-indol-6-yl]prop-2-ynamide,
21) N-(3-chlorophenyl)-3-[2-oxo-3-[phenyl-[4-(pyrrolidin-1-ylmethyl)anilino]methylidene]-1H-indol-6-yl]prop-2-ynamide,
22) N-(3-chlorophenyl)-3-[3-[[4-(dimethylaminomethyl)anilino]-phenylmethylidene]-2-oxo-1H-indol-6-yl]prop-2-ynamide,
23) 3-[2-oxo-3-[phenyl-[4-(pyrrolidin-1-ylmethyl)anilino]methylidene]-1H-indol-6-yl]-N-phenylprop-2-ynamide,
24) 3-[2-oxo-3-[phenyl-[4-(pyrrolidin-1-ylmethyl)anilino]methylidene]-1H-indol-6-yl]-N-pentan-3-ylprop-2-ynamide, and
25) N-(3-fluorophenyl)-3-[2-oxo-3-[phenyl-[4-(pyrrolidin-1-ylmethyl)anilino]methylidene]-1H-indol-6-yl]prop-2-ynamide,
a pharmaceutically acceptable salt of any of the above,
and a combination of any of the above.

3. The method according to claim 1, wherein the anti-IGF antibody is an isolated human antibody molecule, which
a) binds to human IGF-1 and IGF-2 such that
i) binding of IGF-1 and IGF-2 to the IGF-1 receptor is prevented and
ii) IGF-1 receptor-mediated signaling is inhibited,
b) binds to mouse and rat IGF-1 and IGF-2,
c) does not bind to human insulin;
wherein said antibody molecule is selected from the group comprising
i) an antibody molecule that has heavy chain CDRs comprising the amino acid sequences of SEQ ID NO:1 (CDR1), SEQ ID NO:2 (CDR2) and SEQ ID NO:3 (CDR3) and that has light chain CDRs comprising the amino acid sequences of SEQ ID NO:4 (CDR1), SEQ ID NO:5 (CDR2) and SEQ ID NO:6 (CDR3); or
ii) an antibody molecule that has heavy chain CDRs comprising the amino acid sequences of SEQ ID NO:11 (CDR1), SEQ ID NO:12 (CDR2) and SEQ ID NO:13 (CDR3) and that has light chain CDRs comprising the amino acid sequences of SEQ ID NO:14 (CDR1), SEQ ID NO:15 (CDR2) and SEQ ID NO:16 (CDR3); or
iii) an antibody molecule that has heavy chain CDRs comprising the amino acid sequences of SEQ ID NO:21 (CDR1), SEQ ID NO:22 (CDR2) and SEQ ID NO:23 (CDR3) and that has light chain CDRs comprising the amino acid sequences of SEQ ID NO:24 (CDR1), SEQ ID NO:25 (CDR2) and SEQ ID NO:26 (CDR3); or a combination of any of the above.

4. The method according to claim 1, wherein the anti-IGF antibody is an anti-IGF antibody molecule having heavy chain CDRs comprising the amino acid sequences of SEQ ID NO:1 (CDR1), SEQ ID NO:2 (CDR2) and SEQ ID NO:3 (CDR3) and light chain CDRs comprising the amino acid sequences of SEQ ID NO:4 (CDR1), SEQ ID NO:5 (CDR2) and SEQ ID NO:6 (CDR3).

5. The method according to claim 1, wherein the anti-IGF antibody is an anti-IGF antibody molecule having heavy chain CDRs comprising the amino acid sequences of SEQ ID NO:11 (CDR1), SEQ ID NO:12 (CDR2) and SEQ ID NO:13 (CDR3) and light chain CDRs comprising the amino acid sequences of SEQ ID NO:14 (CDR1), SEQ ID NO:15 (CDR2) and SEQ ID NO:16 (CDR3).

6. The method according to claim 1, wherein the anti-IGF antibody is an anti-IGF antibody molecule having heavy chain CDRs comprising the amino acid sequences of SEQ ID NO:21 (CDR1), SEQ ID NO:22 (CDR2) and SEQ ID NO:23 (CDR3) and has light chain CDRs comprising the amino acid sequences of SEQ ID NO:24 (CDR1), SEQ ID NO:25 (CDR2) and SEQ ID NO:26 (CDR3).

7. The method according to claim 3, wherein the antibody molecule has a variable heavy chain comprising the amino acid sequence of SEQ ID NO:8.

8. The method according to claim 3, wherein the antibody molecule has a variable light chain comprising the amino acid sequence of SEQ ID NO:10.

9. The method according to claim 3, wherein the antibody molecule has a variable heavy chain comprising the amino acid sequence of SEQ ID NO:18.

10. The method according to claim 3, wherein the antibody molecule has a variable light chain comprising the amino acid sequence of SEQ ID NO:20.

11. The method according to claim 3, wherein the antibody molecule has a variable heavy chain comprising the amino acid sequence of SEQ ID NO:28.

12. The method according to claim 3, wherein the antibody molecule has a variable light chain comprising the amino acid sequence of SEQ ID NO:30.

13. The method according to claim 1, wherein the antibody molecule comprises a heavy chain constant region selected from the group consisting of IgG1, IgG2, IgG3, IgG4, IgM, IgA and IgE constant regions.

14. The method according to claim 13, wherein said heavy chain constant region is IgG1 comprising the amino acid sequence of SEQ ID NO:32.

15. The method according to claim 1, wherein the antibody molecule comprises a light chain constant region which is IgX.

16. The method according to claim 15, wherein the light chain constant region comprises the amino acid sequence of SEQ ID NO:34.

17. The method according to claim 7, wherein the antibody molecule has
a) a heavy chain comprising the amino acid sequence of SEQ ID NO:35, and
b) a light chain comprising the amino acid of SEQ ID NO:36.

18. The method according to claim 9, wherein the antibody molecule has
a) a heavy chain comprising the amino acid sequence of SEQ ID NO:37, and
b) a light chain comprising the amino acid sequence of SEQ ID NO:38.

19. The method according to claim 11, wherein the antibody molecule has
a) a heavy chain comprising the amino acid sequence of SEQ ID NO:39, and
b) a light chain comprising the amino acid sequence of SEQ ID NO:40.

20. The method according to claim 1, wherein the antibody molecule is a Fab, F(ab′)₂, or single chain Fv fragment.

21. The method according to claim 1, wherein the anti-IGF antibody is an antibody molecule binding to a nonlinear epitope within IGF-1 comprising the amino acid sequences LCGAELVDALQFVCGDR (SEQ ID NO:41) and CCFRSCDLRRLEM (SEQ ID NO:42) of human IGF-1 (SEQ ID NO:43).

22. The method according to claim 21, wherein the anti-IGF antibody is an antibody molecule making contact with at least 8 amino acids within the amino acid sequence LCGAELVDALQFVCGDR (SEQ ID NO:41), and at least 10 amino acids within amino acid sequence CCFRSCDLRRLEM (SEQ ID NO:42) of human IGF-1 (SEQ ID NO:43).

23. The method according to claim 21, wherein the anti-IGF antibody is an antibody molecule making contact with Leu (5), Cys (6), Glu (9), Leu (10), Asp (12), Ala (13), Phe (16), Val (17), Arg (21), Cys (47), Cys (48), Phe (49), Ser (51), Cys (52), Asp (53), Leu (54), Arg (55), Leu (57), and Glu (58) of human IGF-1 (SEQ ID NO:43), as determined by X-ray crystallography.

24. The method according to claim 21, wherein the anti-IGF antibody molecule is the antibody molecule of any one of claim 6, 11, 12, or 19.

25. The method according to claim 1 wherein the dual Aurora kinase/MEK inhibitor is selected from
1) N-ethyl-3-[3-[[4-(4-methylpiperazin-1-yl)anilino]-phenylmethylidene]-2-oxo-1H-indol-6-yl]prop-2-ynamide,
2) N-(2,2-difluoroethyl)-3-[3-[[4-(dimethylaminomethyl)anilino]-phenylmethylidene]-2-oxo-1H-indol-6-yl]prop-2-ynamide,
3) N-(2,2-difluoroethyl)-3-[2-oxo-3-[phenyl-[4-(pyrrolidin-1-ylmethyl)anilino]methylidene]-1H-indol-6-yl]prop-2-ynamide,
4) N-(2-fluoroethyl)-3-[2-oxo-3-[phenyl-[4-(pyrrolidin-1-ylmethyl)anilino]methylidene]-1H-indol-6-yl]prop-2-ynamide,
5) N-ethyl-3-[2-oxo-3-[phenyl-[4-(pyrrolidin-1-ylmethyl)anilino]methylidene]-1H-indol-6-yl]prop-2-ynamide,
6) 3-[3-[[4-(dimethylaminomethyl)anilino]-phenylmethylidene]-2-oxo-1H-indol-6-yl]-N-ethylprop-2-ynamide,
7) N-cyclobutyl-3-[3-[[4-(4-methylpiperazin-1-yl)anilino]-phenylmethylidene]-2-oxo-1H-indol-6-yl]prop-2-ynamide,
8) N-cyclopropyl-3-[3-[[4-(dimethylaminomethyl)anilino]-phenylmethylidene]-2-oxo-1H-indol-6-yl]prop-2-ynamide,
9) 3-[3-[[4-(dimethylaminomethyl)anilino]-phenylmethylidene]-2-oxo-1H-indol-6-yl]-N-phenylprop-2-ynamide,
10) N-cyclopentyl-3-[3-[[4-(dimethylaminomethyl)anilino]-phenylmethylidene]-2-oxo-1H-indol-6-yl]prop-2-ynamide,
11) N-cyclopentyl-3-[3-[[4-(4-methylpiperazin-1-yl)anilino]-phenylmethylidene]-2-oxo-1H-indol-6-yl]prop-2-ynamide,
12) N-cyclobutyl-3-[3-[[4-(dimethylaminomethyl)anilino]-phenylmethylidene]-2-oxo-1H-indol-6-yl]prop-2-ynamide,
13) 3-[3-[[4-(dimethylaminomethyl)anilino]-phenylmethylidene]-2-oxo-1H-indol-6-yl]-N-(2-hydroxyethyl)prop-2-ynamide,
14) 3-[3-[[4-(dimethylaminomethyl)anilino]-phenylmethylidene]-2-oxo-1H-indol-6-yl]-N-propan-2-ylprop-2-ynamide,
15) 3-[2-oxo-3-[phenyl-[4-(pyrrolidin-1-ylmethyl)anilino]methylidene]-1H-indol-6-yl]-N-propan-2-ylprop-2-ynamide,
16) N-(2-hydroxyethyl)-3-[2-oxo-3-[phenyl-[4-(pyrrolidin-1-ylmethyl)anilino]methylidene]-1H-indol-6-yl]prop-2-ynamide,
17) N-(2-fluorophenyl)-3-[2-oxo-3-[phenyl-[4-(pyrrolidin-1-ylmethyl)anilino]methylidene]-1H-indol-6-yl]prop-2-ynamide,
18) 3-[3-[[4-(dimethylaminomethyl)anilino]-phenylmethylidene]-2-oxo-1H-indol-6-yl]-N-[(2S)-1-hydroxypropan-2-yl]prop-2-ynamide,
19) N-[(2S)-1-hydroxypropan-2-yl]-3-[2-oxo-3-[phenyl-[4-(pyrrolidin-1-ylmethyl)anilino]methylidene]-1H-indol-6-yl]prop-2-ynamide,
20) N-[(2R)-butan-2-yl]-3-[2-oxo-3-[phenyl-[4-(pyrrolidin-1-ylmethyl)anilino]methylidene]-1H-indol-6-yl]prop-2-ynamide,
21) N-(3-chlorophenyl)-3-[2-oxo-3-[phenyl-[4-(pyrrolidin-1-ylmethyl)anilino]methylidene]-1H-indol-6-yl]prop-2-ynamide,
22) N-(3-chlorophenyl)-3-[3-[[4-(dimethylaminomethyl)anilino]-phenylmethylidene]-2-oxo-1H-indol-6-yl]prop-2-ynamide,
23) 3-[2-oxo-3-[phenyl-[4-(pyrrolidin-1-ylmethyl)anilino]methylidene]-1H-indol-6-yl]-N-phenylprop-2-ynamide,
24) 3-[2-oxo-3-[phenyl-[4-(pyrrolidin-1-ylmethyl)anilino]methylidene]-1H-indol-6-yl]-N-pentan-3-ylprop-2-ynamide, and
25) N-(3-fluorophenyl)-3-[2-oxo-3-[phenyl-[4-(pyrrolidin-1-ylmethyl)anilino]methylidene]-1H-indol-6-yl]prop-2-ynamide,
a pharmaceutically acceptable salt of any of the above,
and a combination of any of the above,
and
i) the anti-IGF antibody has
a) a heavy chain comprising the amino acid sequence of SEQ ID NO:35, and
b) a light chain comprising the amino acid of SEQ ID NO:36, or
ii) the anti-IGF antibody molecule has
a) a heavy chain comprising the amino acid sequence of SEQ ID NO:37, and
b) a light chain comprising the amino acid of SEQ ID NO:38, or
iii) the anti-IGF antibody molecule has
a) a heavy chain comprising the amino acid sequence of SEQ ID NO:39, and
b) a light chain comprising the amino acid of SEQ ID NO:40.
